(19) United States
(12) Patent Application Publication
(10) Pub. No.: US 2021/0017174 A1
(43) Pub. Date: Jan. 21, 2021

(54) IDENTIFICATION AND USE OF ERK5 INHIBITOR

(71) Applicants: Bayer Aktiengesellschaft, Leverkusen (DE); Bayer Pharma Aktiengesellschaft, Berlin (DE)

(72) Inventors: Duy NGUYEN, Berlin (DE); Lars WORTMANN, Biberbach an der Riss (DE); Adelaide Clara FARIA ALVARES DE LEMOS, Berlin (DE); Ulf BOEMER, Glienicke (DE); Detlev SUELZLE, Berlin (DE); Simon HOLTON, Berlin (DE); Christian LECHNER, Berlin (DE)

(21) Appl. No.: 16/978,060

(22) PCT Filed: Mar. 1, 2019
(86) PCT No.: PCT/EP2019/055160

§ 371 Date: Sep. 3, 2020

(30) Foreign Application Priority Data
Mar. 7, 2018 (EP) 18160540.3

(51) Int. Cl. C07D 471/04; C07D 401/04; C07D 401/14

(52) U.S. Cl. CPC C07D 471/04; A61K 45/06; C07D 401/14; C07D 401/04

(57) ABSTRACT

The present invention covers heterocyclic compounds of general formula (I)

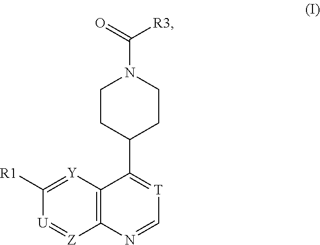

in which T, U, Y, Z, R1 and R3 are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of diseases, in particular of cancer disorders, as a sole agent or in combination with other active ingredients.

The present invention covers heterocyclic compounds of general formula (I)

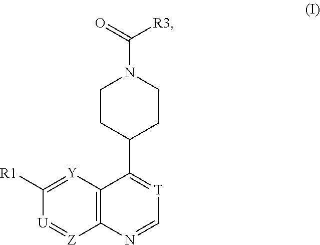

in which T, U, Y, Z, R1 and R3 are as defined herein, methods of preparing said compounds, intermediate compounds useful for preparing said compounds, pharmaceutical compositions and combinations comprising said compounds and the use of said compounds for manufacturing pharmaceutical compositions for the treatment or prophylaxis of diseases, in particular of cancer disorders, as a sole agent or in combination with other active ingredients.

### BACKGROUND

The extracellular signal-regulated kinase 5 (ERK5, also known as big MAP kinase 1, BMK1) protein, encoded by the MAPK7 gene, is a member of the mitogen-activated protein kinase (MAPK) family. The ERK5 signaling cascade can be activated by environmental stresses, mitogens and cytokines. These stimuli activate MEKK2 and MEKK3, which are able to phosphorylate and activate MEK5. Once activated, MEK5 phosphorylates the TEY motif in the activation loop of the ERK5 kinase domain, thereby leading to ERK5 activation. (for review see Hoang et al, 2017. Cancer letters; Drew et al, 2012. Biochimica et Biophysica Acta; Nithianandarajah-Jones et al, 2012. Cellular Signalling).

ERK5 contains an N-terminal kinase domain, which is similar to that of ERK1/2. Additionally, ERK5 has an extended C-terminal region containing a nuclear localization signal (NLS) and a transcriptional activation domain (TAD) (Kasler et al, 2000. Mol Cell Biol; Nithianandarajah-Jones et al, 2012. Cellular Signalling). It has been shown that, in its unphosphorylated form, ERK5 assumes a closed conformation due to molecular interactions between its N- and C-terminus. Upon phosphorylation by MEK5 and consequent activation, ERK5 autophosphorylates its C-terminal tail, thereby disrupting the intramolecular interaction and inducing a conformational change that exposes the NLS and shuttles ERK5 to the nucleus (Erazo et al, 2013. Mol Cell Biol; Kondoh et al, 2006. Mol Cell Biol; Morimoto et al, 2007. J Biol Chem; Simoes et al, 2016. Drug Discovery Today). Importantly, ERK5 signaling is highly dependent on its kinase activity, which is necessary to directly phosphorylate and activate downstream targets (e.g, cyclin D1, MEF2C, c-Fos, Fra-1), but also required to activate the TAD and enhance transcriptional activation (Morimoto et al, 2007. J Biol Chem; Mulloy et al, 2003. Oncogene; Kasler et al, 2000. Mol Cell Biol; Kato et al, 1997. EMBO Journal; Teresawa et al, 2003. Genes to Cells).

ERK5 is a key integrator of cellular signal transduction and it has been shown to play a role in various cellular processes such as proliferation, differentiation, apoptosis and cell survival. Several studies have demonstrated that silencing ERK5 with siRNA or shRNA decreases the proliferation and increases cell death in different tumor models, thereby highlighting the potential of ERK5 as a therapeutic target in cancer (Hoang et al, 2017. Cancer letters; Drew et al, 2012. Biochimica et Biophysica Acta; Simoes et al, 2016. Drug Discovery Today). Of note, many cancer types (e.g., sarcoma and hepatocellular carcinoma) display genomic ERK5 amplifications, while others exhibit constitutive activation of ERK5 (e.g., breast cancer with ErbB2 overexpression), rendering them particularly sensitive to ERK5 depletion (Esparis-Ogando et al, 2002. Mol Cell Biol; Shukla et al, 2013. Clin Cancer Res; Zen et al, 2009. Genes, Chromosome & Cancer; Gavine et al, 2015. BMC cancer).

### State of the Art Analysis

The therapeutic potential of targeting ERK5 resulted in different attempts to develop ERK5 kinase inhibitors over the recent years. Benzo[e]pyrimido-[5,4-b]diazepine-6(11H)-one based XMD8-92, has been extensively used and showed promising in vitro and in vivo anti-tumor efficacy (Deng et al, 2011. ACS Med Chem Lett; Yang et al, 2010. Cancer Cell). However, a recent study demonstrated that the biological activity of XMD8-92 derived from an off-target activity on bromodomains (BRDs) (Lin et al, 2016. PNAS).

Pyrrolecarboxamide derivatives were also disclosed as ERK5 inhibitors (I. Hardcastle et al., ACS Comb. Sci. 2016, 18, 444-455, WO 2016/042341). In addition, nicotine and benzothiazoles derivatives were mentioned as ERK5 inhibitors as well, although the activity reported were modest (I. Hardcastle et al., ACS Comb. Sci. 2016, 18, 444-455).

Dual MEK5/ERK5 1-indolin-2-one-based inhibitors BIX 02188 and BIX 02189 have been reported with only modest activity on ERK5 (R. J. Tatake et al. Biochem. Biophys. Res. Commun. 2008, 377 (1), 120-125).

To this day, a potent and selective ERK5 inhibitor has not been available.

Compounds such as 4-[1-(2,5-dimethyl-4-oxazolyl)piperidin-4-yl]-quinoline (CAS No: 2094348-85-1), 4-[1-(4-methyl-1H-pyrrol-3-yl)piperidin-4-yl]-quinoline (CAS No: 2094289-44-6), 4-[1-(2,4-dimethyl-5-oxazolyl)piperidin-4-yl]-4-pyrido[2,3-d]pyrimidine (CAS No 1381384-21-9), with 5-membered heteroaromatic ring attached to the amide moiety are commercially available.

WO 2006/13572 claims alkylquinoline and alkylquinazoline derivatives as kinase modulators, particularly as inhibitors of FLT3, ckit and TrkB. However, claims were restricted to urea derivatives.

WO 2016/073774 relates to indoleamine-2,3-dioxygenase inhibitors. WO 2015/054572 relates to inhibitors of G12C mutant KRAS protein. WO 2010/023161 relates to aryl- and heteroarylcarbonyl derivatives of substituted nortropanes as inhibitors of 11 beta-hydroxysteroid dehydrogenase (HSD) 1. WO 2007/071055 relates to compositions which modulate the activity of gated ion channels. WO 1999/065867 relates to cyclic hydroxamic acids as metalloproteinase inhibitors. EP 1 106 605 A1 relates to alpha 1B-adrenergic receptor antagonists.

Specifically disclosed compounds in the disclosures mentioned above are not included in general formula (I) of the present invention.

As mentioned supra, the present invention covers heterocyclic compounds of general formula (I)

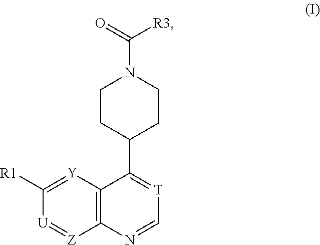

in which T, U, Y, Z, R1 and R3 are as defined herein.

To date, heterocyclic compounds of general formula (I) of the present invention have not been described in the prior art.

It has now been found, and this constitutes the basis of the present invention, that the compounds of general formula (I) of the present invention have surprising and advantageous properties.

In particular, the compounds of the present invention have surprisingly been found to effectively inhibit ERK5 for which data are given in biological experimental section and may therefore be used for the treatment or prophylaxis of cancer disorders, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers, for example.

### DESCRIPTION OF THE INVENTION

In accordance with a first aspect, the present invention covers compounds of general formula (I):

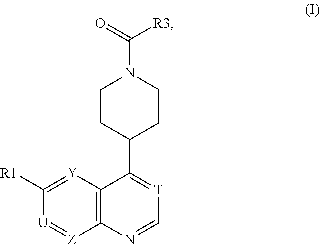

in which
T represents CH, CR4, or N;
Y represents CH, CR4, or N;
R1 represents a hydrogen atom, a C₁-C₆-alkyl, —CH3, or C₁-C₆-alkoxy, —OCH₃ group;
U represents CH, CR2, or N;
R2 represents a hydrogen atom, a halogen atom, a bromine atom, a hydroxyl, cyano, a C₁-C₆-alkyl, —CH₃, C₂-C₆-alkenyl, C₃-C₈-cycloalkyl, C₄-C₈-cycloalkenyl, phenyl, 4- to 7-membered heterocycloalkyl, 5- to 8-membered heterocycloalkenyl, heteroaryl, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NR5R6, —C(═O)NH₂, —C(═O)NHCH₃, —C(═O)N(CH₃)₂, —C(═O)NHC₆H₅, —C(═O)OR7-C(═O)OCH₃, —NH₂, —N(H)C(═O)—C₁-C₆-alkyl, —NHC(═O)CH₃, —N(H)—C₃-C₈-cycloalkyl, —N(H)-4- to 7-membered heterocycloalkyl, —N(H)-1-methylpiperidin-4-yl, —N(H)-phenyl, —N(H)S(═O)₂—C₁-C₆-alkyl, —N(H)S(═O)₂CH₃, C₁-C₆-alkoxy which is optionally substituted with a hydroxyl, 4-membered heterocycloalkyl, —NR5R6, or —NHC(═O)—C₁-C₆-alkyl substituent, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃, —O—C₃-C₈-cycloalkyl, —O-(4- to 7-membered heterocycloalkyl), —O-(tetrahydro-2H-pyran-4-yl), C₁-C₆-haloalkoxy, —O(CH₂)CHF₂;
Z represents CH, CR4, —C—C₁-alkyl, or N;
R3 represents a phenyl or pyridyl ring which is optionally substituted once or twice identically or differently with a substituent selected from a halogen atom, a fluorine atom, a chlorine atom, a bromine atom, a hydroxyl, cyano, C₁-C₆-alkyl which is optionally substituted with a C₁-C₆-alkoxy- or C₁-C₆-haloalkoxy-substituent, C1-C₃-C₄-alkyl, trifluoromethyloxymethyl, trifluoroethyloxymethyl group, C₁-C₆-haloalkyl, C₁-trifluoroalkyl, C₂-C₆-alkenyl, C₃-alkenyl, C₃-C₈-cycloalkyl, —C(═O)NR6R7, —NH₂, —NH—C(═O)—C₁-C₆-alkyl, —NHC(═O)CH₃, C₁-C₆-alkoxy which is optionally substituted with a hydroxyl or C₁-C₆-alkyl substituent, C₁- C₂- C₃- C₄-alkoxy, methoxyethoxy, C₁-C₆-haloalkoxy, C₁-trifluoroalkoxy, C₁-difluoroalkoxy, —O—C₃-C₈-cycloalkyl, —O-(4- to 7-membered heterocycloalkyl), C₁-C₆-alkylthio, C₁-C₆-haloalkylthio, C₁-trifluoroalkylthio, or —S(═O)₂—C₁-C₆-alkyl group;
R4 represents a hydrogen atom, or C₁-C₆-alkyl group;
R5, R6 represent, independently from each other
   a hydrogen atom, a C₁-C₆-alkyl or phenyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
   and stereoisomers, tautomers, N-oxides, hydrates, solvates, and salts thereof, and mixtures of same.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings: The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-alkylthio” means a linear or branched, saturated, monovalent group of formula (C₁-C₆-alkyl)-S—, in which the term “C₁-C₆-alkyl” is as defined supra, e.g. a methylthio, ethylthio, n-propylthio, isopropylthio, n-butylthio, sec-butylthio, isobutylthio, tert-butylthio, pentylthio, isopentylthio or n-hexylthio group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₁-C₆-haloalkylthio” means a linear or branched, saturated, monovalent C₁-C₆-alkylthio group, as defined supra, in which one or more of the hydrogen atoms is replaced, identically or differently, with a halogen atom. Particularly, said halogen atom is a fluorine atom. Said C₁-C₆-haloalkylthio group is, for example, fluoromethylthio, difluoromethylthio, trifluoromethylthio, 2,2,2-trifluoroethylthio or pentafluoroethylthio.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo[2.2.2]oct-2-enyl.

The terms “4- to 7-membered heterocycloalkyl” and “4- to 6-membered heterocycloalkyl” mean a monocyclic, saturated heterocycle with 4, 5, 6 or 7 or, respectively, 4, 5 or 6 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “5- to 8-membered heterocycloalkenyl” means a monocyclic, unsaturated, non-aromatic heterocycle with 5, 6, 7 or 8 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly, the heteroaryl group is a pyridyl group.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio.

Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

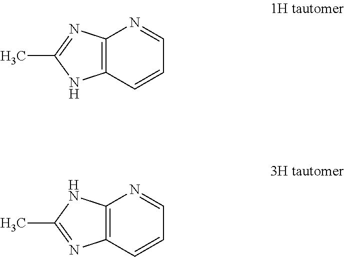

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Nat”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with a second embodiment of the first aspect, the present invention covers compounds of general formula (I), supra, in which:
T represents N;
Y represents CH, or N;
R1 represents a C₁-C₆-alkyl, —CH3, or C₁-C₆-alkoxy, —OCH₃ group;
U represents CR2, or N;
R2 represents a hydrogen atom, a halogen atom, a bromine atom, a hydroxyl, cyano, a C₁-C₆-alkyl, —CH₃, 4- to 7-membered heterocycloalkyl, 5- to 8-membered heterocycloalkenyl, heteroaryl, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, (3R)-3-(dimethylamino)pyrrolidin-1-yl-, —C(═O)NR5R6, —C(═O)NH₂, —C(═O)NHCH₃, —C(═O)N(CH₃)₂, —C(═O)NHC₆H₅, —C(═O)OR7-C(═O)OCH₃, —NH₂, —N(H)C(═O)—C₁-C₆-alkyl, —NHC(═O)CH₃, —N(H)-4- to 7-membered heterocycloalkyl, —N(H)-1-methylpiperidin-4-yl, —N(H)S(═O)₂—C₁-C₆-alkyl, —N(H)S(═O)₂CH₃, C₁-C₆-alkoxy which is optionally substituted with a hydroxyl, 4-membered heterocycloalkyl, —NR5R6, or —NHC(═O)—C₁-C₆-alkyl substituent, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃, —O-(4- to 7-membered heterocycloalkyl), —O-(tetrahydro-2H-pyran-4-yl), C₁-C₆-haloalkoxy, —O(CH₂)CHF₂;
Z represents CH, CR4, —C—C₁-alkyl, or N;
R3 represents a phenyl or pyridyl ring which is optionally substituted once or twice identically or differently with a substituent selected from a halogen atom, a fluorine atom, a chlorine atom, a bromine atom, cyano, C₁-C₆-alkyl which is optionally substituted with a C₁-C₆-alkoxy- or C₁-C₆-haloalkoxy-substituent, C1-C₃-C₄-alkyl, trifluoromethyloxymethyl, trifluoroethyloxymethyl group, C₁-C₆-haloalkyl, C₁-trifluoroalkyl, C₂-C₆-alkenyl, C₃-alkenyl, —NH₂, —NH—C(═O)—C₁-C₆-alkyl, —NHC(═O)CH₃, C₁-C₆-alkoxy which is optionally substituted with a C₁-C₆-alkyl substituent, C₁- C₂- C₃- C₄-alkoxy, methoxyethoxy, C₁-C₆-haloalkoxy, C₁-trifluoroalkoxy, C₁-difluoroalkoxy, or C₁-C₆-haloalkylthio, C₁-trifluoroalkylthio group;
R4 represents a hydrogen atom, or C₁-C₆-alkyl group;
R5, R6 represent, independently from each other
   a hydrogen atom, or a C₁-C₆-alkyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
   and stereoisomers, tautomers, N-oxides, hydrates, solvates, and salts thereof, and mixtures of same.

In accordance with a third embodiment of the first aspect, the present invention covers compounds of general formula (I), supra, in which:
T represents N;
Y represents CH, or N;
R1 represents a —CH3, or —OCH₃ group;
U represents CR2, or N;
R2 represents a hydrogen atom, a bromine atom, a hydroxyl, cyano, —CH₃, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NH₂, —C(═O)NHCH₃, —C(═O)N(CH₃)₂, —C(═O)NHC₆H₅, —C(═O)OCH₃, —NH₂, —NHC(═O)CH₃, —N(H)-1-methylpiperidin-4-yl, —N(H)S(═O)₂CH₃, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃—O-(tetrahydro-2H-pyran-4-yl), or —O(CH₂)CHF₂ group;
Z represents CH, —C—C₁-alkyl, or N;
R3 represents a phenyl or pyridyl ring which is optionally substituted once or twice identically or differently with a substituent selected from a fluorine atom, a chlorine atom, a bromine atom, cyano, C₁-, C₃, or C₄-alkyl, trifluoromethyloxymethyl, trifluoroethyloxymethyl group, C₁-trifluoroalkyl, C₃-alkenyl, —NH₂, —NHC(═O)CH₃, C₁- C₂- C₃- C₄-alkoxy, methoxyethoxy, C₁-trifluoroalkoxy, C₁-difluoroalkoxy, or C₁-trifluoroalkylthio group;
R4 represents a hydrogen atom, or C₁-C₆-alkyl group;
R5, R6 represent, independently from each other
   a hydrogen atom, or a C₁-C₆-alkyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
   and stereoisomers, tautomers, N-oxides, hydrates, solvates, and salts thereof, and mixtures of same.

In accordance with a fourth embodiment of the first aspect, the present invention covers compounds of general formula (I), supra, in which:
T represents N;
Y represents CH, or N;
R1 represents a —CH3, or —OCH₃ group;
U represents CR2;
R2 represents a hydrogen atom, a bromine atom, a hydroxyl, cyano, —CH₃, 3,6-dihydro-2H-pyran-4-yl-, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NH₂, —C(═O)N(CH₃)₂, —C(═O)OCH₃, —NH₂, —N(H)-1-methylpiperidin-4-yl, —N(H)S(═O)₂CH₃, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃—O-(tetrahydro-2H-pyran-4-yl), or —O(CH₂)CHF₂ group;
Z represents CH, or —C—C₁-alkyl group;
R3 represents a phenyl ring which is optionally substituted once or twice identically or differently with a substituent selected from a fluorine atom, a chlorine atom, a bromine atom, C₁-, or C₃-alkyl, C₁-trifluoroalkyl, C₃-alkenyl, —NH₂, —NHC(═O)CH₃, C₁- C₂- C₃- C₄-alkoxy, C₁-difluoroalkoxy, C₁-trifluoroalkoxy, or C₁-trifluoroalkylthio group;
R4 represents a hydrogen atom, or C₁-C₆-alkyl group;
R5, R6 represent, independently from each other
   a hydrogen atom, or a C₁-C₆-alkyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
   and stereoisomers, tautomers, N-oxides, hydrates, solvates, and salts thereof, and mixtures of same.

The present invention covers any sub-combination within any embodiment or aspect of the present invention of compounds of general formula (I), supra.

The present invention covers any sub-combination within any embodiment or aspect of the present invention of intermediate compounds of general formula (2′).

The present invention covers the compounds of general formula (I) which are disclosed in the Example Section of this text, infra.

### Synthesis of the Compounds of the Present Invention

The compounds according to the invention of general formula (I) can be prepared according to the following Schemes 1 to 6. The schemes and procedures described below illustrate synthetic routes to the compounds of general formula (I) of the invention and are not intended to be limiting. It is clear to the person skilled in the art that the order of transformations as exemplified in the schemes can be modified in various ways. The order of transformations exemplified in these schemes is therefore not intended to be limiting. In addition, interconversion of any of the substituents, R1, R2, R3, R4, R5, R6 or R7 can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

The routes for the preparation of compounds of general formula (I) are described in Schemes 1 to 6.

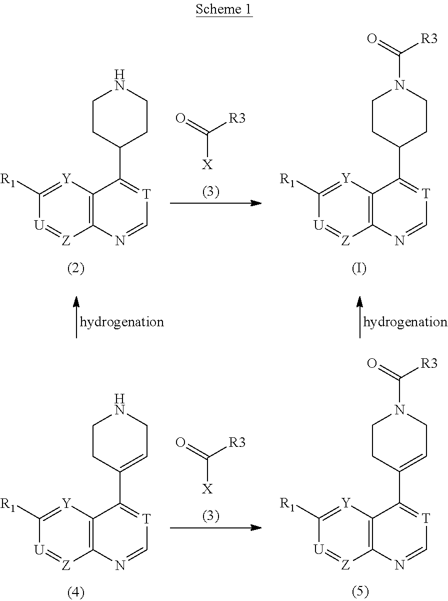

Compounds of formula (I) can be obtained by amide coupling using compounds of formula (2) with compounds of formula (3). Alternatively, amide coupling can also be performed using compounds of formula (4) and compounds of formula (3) to afford compounds of formula (5). Subsequent hydrogenation result in the formation of compounds of formula (I).

Amide coupling of compounds of formula (3) with compounds of formula (2) or with compounds of formula (4) can be achieved when compounds of (3) is an acid chloride (X=Cl), an acid (X=OH), an ester (X=O-alkyl) or an anhydride (X=O—C(O)alkyl/aryl). Compounds of formula (3) are commercially available or synthesize as needed and will be disclosed with specific examples.

Reactions of compounds of formula (2) or of formula (4) with an acid chloride (X=Cl) of formula (3) occur in the presence of a base, such as triethylamine, pyridine, N-ethyl-N,N-diisopropylamine, in an aprotic polar/non polar solvents such as acetonitrile, dichloromethane, 1,2 dichloroethane, chloroform, N,N-dimethylformamide (DMF), 1-methyl-pyrrolidin-2-one (NMP) at ambient or elevated temperatures. Occasionally, small amount of a catalyst, such as N,N-dimethylaminopyridine, also known as DMAP, is added to the reaction. For example, see US2003/232854, WO 2006/117570, WO 2008/40934, WO 2008/64432, WO 2009/23655, WO2007/59613, US2002/99035, US2015/158865 and references therein.

Amide coupling of compounds of formula (2) or of formula (4) with an acid (X=OH) of formula (3) occur in the presence of a base and an appropriate coupling reagent in an aprotic polar/non polar solvent at ambient or elevated temperatures. Suitable amide coupling are, for example, O-(7-aza-1H-benzotriazol-1-yl)-N,N,N′,N′-tetramethyluronium hexafluorphosphate, also called HATU, O-(Benzotriazol-1-yl)-N,N,N′,N′-tetramethyluronium tetrafluoroborate (TBTU), dicyclohexylcarbodiimide, a combination of 1H-benzotriazol and 1-ethyl-3-[3-dimethylamino]carbodiimide hydrochloride or propanephosphonic acid anhydride (T3P). Appropriate bases include, for example, N,N-dimethylaminopyridine, N-ethyl-N,N-diisopropylamine, triethylamine. Solvents used in such amide coupling reaction are, for example, N,N-dimethylformamide (DMF), 1-methyl-pyrrolidin-2-one (NMP), dichloromethane or tetrahydrofuran. For example, see WO2010/11837, WO 2005/115972, WO 2006/52722, US 2007/185148. J. Am. Chem. Soc. 1992, 114, 9327, WO 2010/11837, Org. Lett. 2011, 5048-5051 and references cited therein.

Alternatively, amide coupling of compounds of formula (2) or of formula (4) with ester (X=O.alkyl) or anhydride (X=O.alkyl/aryl) of formula (3) occur in the presence of a base such as triethylamine, N,N-dimethylaminopyridine, N-ethyl-N,N-diisopropylamine or in an aprotic polar/non polar solvent such as dichloromethane, N,N-dimethylformamide or acetonitrile. For selected examples of such transformations, see WO2006/19768, WO2005/103000, Angewandte Chemie—International Edition, 2016, vol. 55, #50 p. 15667-15671 and references cited therein.

Compounds of formula (2) can be obtained by hydrogenation of compounds of formula (4), either by using hydrogen or an alternative hydrogen source such as ammonium formate. The same reaction can also be applied for the transformation of compounds of formula (5) to compounds of formula (I).

Suitable catalysts are for example, palladium or platinum on activated charcoal, palladium oxide hydrate, platinum(IV)oxide. For selected examples of hydrogenation reaction, see WO2014/152013, WO2011/139107, US2012/277220, WO2012/62752 and references cited therein.

As already mentioned, interconversions of any of the substituents according to the definition can occur before and/or after the exemplified transformations

Therefore it could be envisioned that starting from compounds of formula (I), substituents such as R1, R2, R3 and R4 can undergo further transformations to directly results in substituents that are in scope of the present invention or indirectly in the introduction of a new chemical group, which enables further chemical manipulations leading to compounds with new substituents. These modifications can be, but not limited to, introduction of protecting groups, cleavage of protecting groups, alkylation, dealkylation, halogenation, metalation, substitution or other reactions known to the person skilled in the art.

For example, for R1, R2, R3 or R4 is an ester or a substituent having an ester, which can undergo ester hydrolysis in the presence of a base such as lithium hydroxide, potassium hydroxide or sodium hydroxide and in a mixture of solvent such as methanol, ethanol or tetrahydrofuran with water. For example, see WO2008/154563, Bioorganic and Medicinal Chemistry Letters, 2011, vol. 21, #12, p. 3671-3675 and references cited therein.

Benzylic ester can be transformed to carboxylic acid by hydrogenation as mentioned above.

The ester can also be converted to a carboxamide by a reaction with ammonia in a solvent such as methanol or ethanol (see WO2016/114668 and references cited therein).

The resulting carboxylic acid can be converted to amide derivatives by using methods as exemplified above.

In case R1, R2, R3 or R4 is a hydroxide group, this group can be alkylated by a reaction with an alkyl/cycloalkyl halogenide in the presence of a base such as sodium carbonate, potassium carbonate or cesium carbonate in an aprotic polar solvent such as N,N-dimethylformamide (DMF), acetone, acetonitrile. Occasionally, sodium iodide, potassium iodide or tetrabutylammonium iodide is also added to the reaction. For example, see: EP2103607, US2015/73158, WO2012/32546, US2010/297073. Alternatively, alkylation reaction can also be accomplished via the Mitsunobu reaction (Synthesis, 1981, 1-28).

The hydroxide group can be converted to a new functionality, which allows further transformations. Such new functionality includes, but not limited to, a triflate group or a nonaflate group, which can be used as a suitable leaving group in subsequent transition metal catalyzed/mediated reaction of Hartwig/Buchwald-type or Suzuki-type.

Triflate formation can be accomplished by using trifluoromethylsulfonic anhydride or 1,1,1-trifluoro-N-phenyl-N-[(trifluoromethyl)sulfonyl]methanesulfonamide in the presence of a base such as triethylamine, pyridine, occasionally with addition of 4-(N,N-dimethylamino)pyridine. Suitable solvents are, for example, dichloromethane or tetrahydrofuran. For example, see: Chemistry—A European Journal, 2013, vol. 19, #10 p. 3504-3511, WO2010/45258. Tetrahedron Asymmetry, 2001, vol. 12, #15 p. 2147-2152, US2005/209166; US2010/256092 and references cited therein.

Nonaflate formation can be obtained by using nonafluoro-n-butanesulfonyl fluoride in the presence of a base such as potassium carbonate, triethylamine, N-ethyl-N,N-diisopropylamine in a solvent such as dichloromethane, 1,2-dichloroethane, acetonitrile or tetrahydrofuran, N,N-dimethyl formamide. For example, see US2012/190660, Journal of Organic Chemistry, 2011, vol. 76, #11 p. 4552-4563, Organic Letters, 2013, vol. 15, #2 p. 374-377, WO2004/87156 and references cited therein.

The Suzuki-type reaction is a valuable synthesis method for C—C bond formation. Starting from an aryl halogenide, aryl triflate or aryl nonaflate and an organo boronic acid or the corresponding boronic ester, C—C bond formation can occur in the presence of a catalyst/ligand system and a base. Suitable catalysts are, for example, bis(diphenylphosphino)ferrocene]dichloropalladium(II), tetrakis(triphenylphosphine) palladium⁽⁰⁾, bis(dibenzylideneacetone)-palladium. Bases used in Suzuki-type reactions are, for example, potassium phosphate, potassium carbonate, triethylamine, or cesium fluoride, Suitable solvents are, for example, toluene, 1,4-dioxane, acetonitrile, N,N-dimethyl formamide or butan-1-ol. For selected examples, see WO2005/73205, WO2008/130320, WO2006/55625, Bioorganic and Medicinal Chemistry Letters, 2012, vol. 22, #17 p. 5618-5624, WO2005/73205, WO2009/111056. EP 2394987, US 2014/275025 and references cited therein.

C—N bond formation via Hartwig/Buchwald-type can be obtained by a reaction of a suitable aryl halogenide, aryl triflate or aryl nonaflate with an amine in the presence of a suitable catalyst/ligand system and a base. Selected suitable conditions are, for example, palladium diacetate/2,2′-bis-(diphenylphosphino)-1,1′-binaphthyl with cesium carbonate in tetrahydrofuran (WO2011/75630), toluene (US2010/286215), 1,4-dioxane (WO2010/136778), with sodium-t-butanolate in toluene (US2005/43309), tris-(dibenzylideneacetone)dipalladium⁽⁰⁾;2,2′-bis-(diphenylphosphino)-1,1′-binaphthyl with sodium t-butanolate in toluene, tetrahydrofuran (WO2009/37220), tris-(dibenzylideneacetone)dipalladium⁽⁰⁾ with (5-diphenylphosphanyl-9,9-dimethyl-xanthen-4-yl)-diphenyl-phosphane and cesium carbonate in 1,4-dioxane (WO2010136778).

In addition, C—N bond formation can also be obtained by a reaction of an aryl halogenide, aryl triflate or aryl nonaflate with an amide or lactam. Selected suitable conditions reported in literature are caesium carbonate; tris-(dibenzylideneacetone)-dipalladium⁽⁰⁾; (5-diphenylphosphanyl-9,9-dimethyl-xanthen-4-yl)-diphenylphosphane, also known as Xantphos, in 1,4-dioxane (US 2007/21408), same condition reported with palladium diacetate as catalyst (WO2007/93364), potassium carbonate, trans-1,2-diaminocyclohexane; copper(I) iodide in 1,4-dioxane (WO 2003/90912) or potassium carbonate; copper(I) iodide; N,N′-dimethylethylenediamine in acetonitrile (WO2011/70039).

Starting from an aryl halogenide or aryl triflate, introduction of an ester group can be achieved by a carbonylation reaction under carbon monoxide atmosphere in the presence of a catalyst. Selected conditions are triethylamine, (1,1′-bis(diphenyl-phosphino)ferrocene)palladium(II) dichloride in ethanol (WO2004/56769), sodium acetate, (1,1′-bis(diphenylphosphino)ferrocene)palladium(II) dichloride in methanol (WO2007/2181), 1,1′-bis-(diphenylphosphino)ferrocene; palladium diacetate; triethylamine in N,N-dimethyl formamide (US2009/247567), (bis(diphenylphosphino)-propane; triethylamine; palladium diacetate in N,N-dimethyl-formamide (WO 2005/51298). The ester can undergo further transformation as described above.

Further interconversion of substituents includes the removal of a protecting group.

For example, debenzylation can be achieved by hydrogenation reactions as described above or under acidic condition, for example by using trifluoroacetic acid (WO2014/15147).

Deprotection of tert-butylcarbamate group (Boc) can be obtained using trifluoroacetic acid in dichloromethane, or a mixture of hydrogen chloride and acetic acid, or hydrogen chloride in 1,4-dioxane and acetone or dichloromethane. For example, see US2006/293341, WO2005/30732, WO2008/40934, WO2007/91694 and WO2004/67516 and references cited therein.

Demethylation of an aryl/heteroaryl methyl ether can be obtained, for example, by using boron tribromide, boron trichloride in dichloromethane (US2011/312995, US2017/182051), aluminium chloride, n-octanethiole in dichloromethane (EP 1731505) or hydrogen chloride in ethanol (Med Chem Comm, 2017, vol. 8, #5 p. 907-916) or sodium thiomethoxide in N,N-dimethylformamide (WO2005/54191).

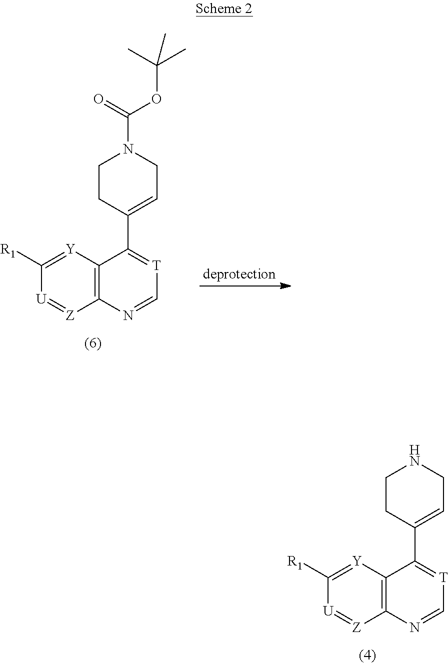

Compounds of formula (4) can be obtained by removal of the tert-butylcarbamate group (Boc) (Scheme 2). Examples of methods for the removal of the Boc-group were described above.

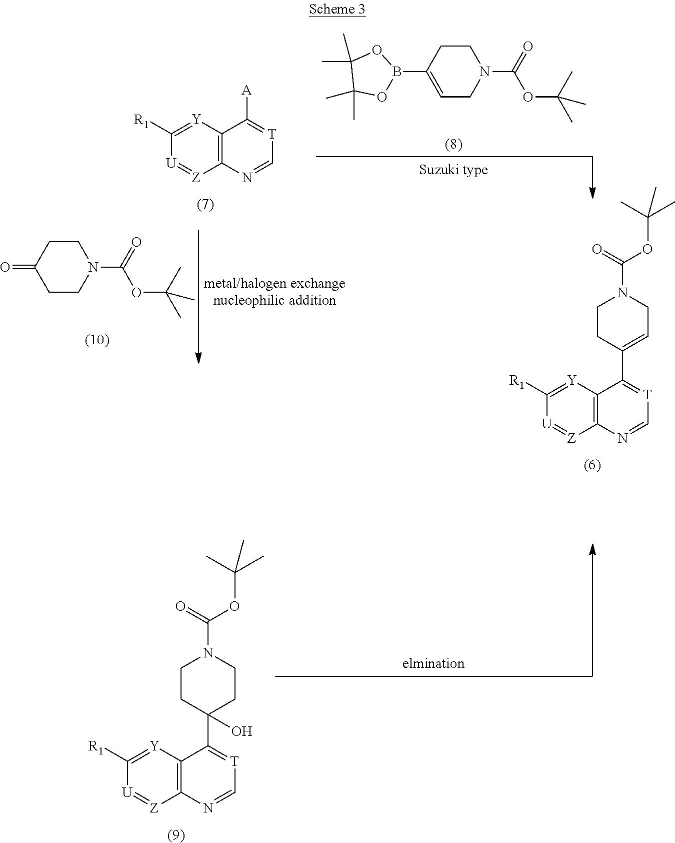

Compounds of formula (6) can be obtained by a Suzuki-type reaction of compounds of formula (7) with compounds of formula (8), in which A is a halogen, a triflate group or a nonaflate group (Scheme 3).

Conditions for Suzuki-type reaction have been described before.

Alternatively, compounds of formula (7) can undergo a metal/halogen exchange reaction and subsequent nucleophilic addition with compounds of formula (10) to afford compounds of formula (9) Suitable reagents used in the metal/halogen exchange reaction are, for example, organo lithium compounds such as n-butyllithium or tert-butyllithium and organo magnesium compounds of Grignard type such as isopropylmagnesium chloride. Solvents used are, for example tetrahydrofuran or diethylether. Reactions were mostly performed at low temperatures when using organo lithium compounds. For example, see WO2016/73770, US2012/71461 and WO 2014/74422 and examples cited therein.

Elimination of the hydroxy group of the compounds of formula (9) results in the formation of the compounds of formula (6). Elimination reaction can be performed under acidic condition, such as in the presence of sulfuric acid in hexane (US2006/229318) or of toluene-4-sulfonic acid in toluene (WO2014/74422). Alternatively, the same transformation can be accomplished when using dehydrating reagent such as bis-[α,α-bis(trifluoromethyl)benzyloxy]diphenylsulfur (CAS 32133-82-7), also known as Martin sulfurane, in dichloromethane (Aldrichimica Acta 18, 81-81, (1985)).

Compounds of formula (8) are either commercially available, or can be obtained from compounds of formula (10) by deprotonation, followed by the formation of the triflate group (see for examples: WO 2012/27341, WO 2010/5783, WO2007/88514) and subsequent borylation reaction (see for examples: WO2004/58727, US2004/8259; Journal of the American Chemical Society, 2009, vol. 131, #28 p. 9612-9613).

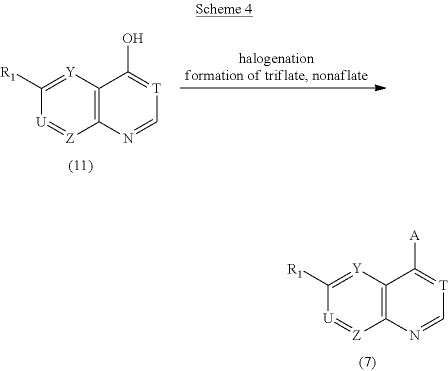

Compounds of formula (7), in which A is halogen, a triflate or a nonaflate group, can be obtained by a halogenation reaction, a formation of triflate or a nonaflate group, starting from compounds of formula (11).

Selected conditions for the formation of a triflate or nanoflate group have been described above. Starting from compounds of formula (11), halogenation can be obtained in the presence of a halogenation transfer reagent such as, for example, thionyl chloride, trichlorophosphate, phosphorous tribromide without any solvent or in solvents such as, for example, acetonitrile, N,N-dimethyl formamide, occasionally in the presence of a base such as N-ethyl-N,N-diisopropylamide, mostly at elevated temperatures. See for selected examples US2006/63805, EP1724268, WO2015/145369, WO2007/16610, WO2010/81874.

Starting from compounds of formula (7) with A=Cl, transfer of halogenation to compounds with A=Br, I is also possible by using, for example, trimethylsilyl bromide acetonitrile (Bioorganic and Medicinal Chemistry, 2012, vol. 20, #2 p. 1076-1089), hydrogen bromide in acetic acid (US2011/105520), phosphorus(V) oxybromide in toluene (WO 2016210036), hydrogen iodide, sodium iodide in water (WO 2005/47279)

Compounds of formula (11), with T=N, can be obtained in various ways. Scheme 5 provides an overview on potential synthesis routes.

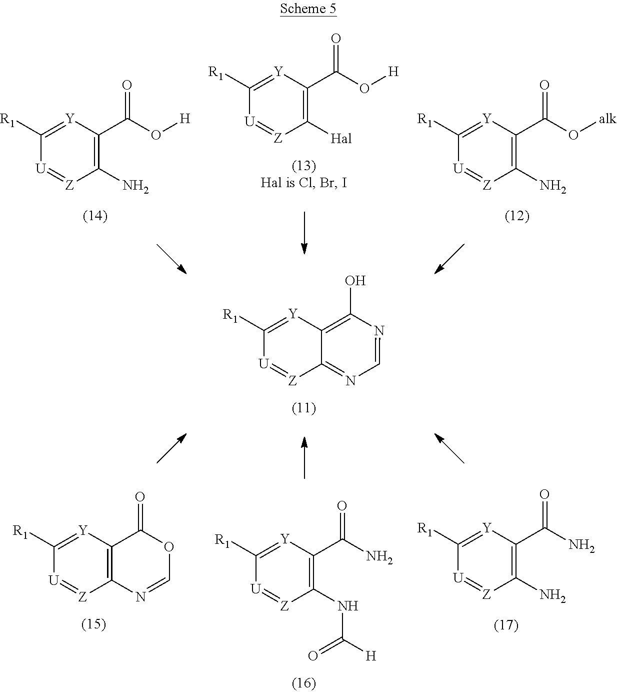

Starting from compounds of formula (12) (either commercially available or described in the literature) can be converted to the corresponding quinazoline of formula (11) in analogy to literature procedures. For selected examples, see WO2008/33747, EP1477481, EP1218357, European Journal of Medicinal Chemistry, 2015, vol. 101, p. 462-475 and references therein.

Alternatively, compounds of formula (13) (either commercially available or described in the literature) can be converted to the corresponding quinazoline (11) in analogy to literature procedures. Typically derivative (13) is reacted with formamidine, copper metal, a base such as for example sodium hydroxide, cesium carbonate in water or in an organic solvent such as for example N,N-dimethyl formamide (DMF) at elevated temperature. For example, see CN103864702, Applied Organometallic Chemistry, 2014, vol. 28, #9 p. 661-665 and references therein.

Alternatively, compounds of formula (14) (either commercially available or described in the literature) can be converted to the corresponding quinazoline (11) in analogy to literature procedures. For example, compounds of formula (14) can undergo reaction with trimethoxymethane in the presence of ammonium acetate in acetonitrile (EP1477481), or with formamide and formamidine acetate (WO2013/96194) or formamide (U.S. Pat. No. 6,184,225), or formamide and ammonium acetate (WO2008/33747).

Alternatively, benzoxazinone derivatives of formula (15) (either commercially available or can be prepared in analogy to literature procedures) can be converted to the corresponding quinazoline (11) in analogy to literature procedures. For example, compounds of formula (15) can be converted to compounds of formula (11) with ammonia hydroxide and ammonium acetate at elevated temperatures (WO2012/69146). Reactions using formamide have also been described (European Journal of Medicinal Chemistry, 2011, vol. 46, #5 p. 1706-1712).

Alternatively benzoic acid amide derivatives of formula (16) (either commercially available or described in the literature) can be converted to the corresponding quinazoline (11) in analogy to literature procedures. For example, in the presence of a base such as sodium hydroxide, cyclization occurs to afford compounds of formula (11) (US2008/207614) or reaction occurs in Kugelrohr apparatus at elevated temperatures without additional base (U.S. Pat. No. 5,990,116).

Alternatively amino benzoic acid amide derivatives of general formula (17) (either commercially available or described in the literature) can be converted to the corresponding quinazoline (11) in analogy to literature procedures. For example, conversions of compounds of formula (17) with triethoxymethane (WO2008/23161), with formic acid at elevated temperatures (WO2013/100632), or with N-({[(E)-(dimethylamino)methylidene]amino}methylidene)-N-methylmethanaminium chloride in dioxane in the presence of sodium acetate and acetic acid (EP1119567) have been described.

Compounds of formula (11), with T=CR4, can be obtained in various ways. Selected examples on the synthesis route are provides in Scheme 6.

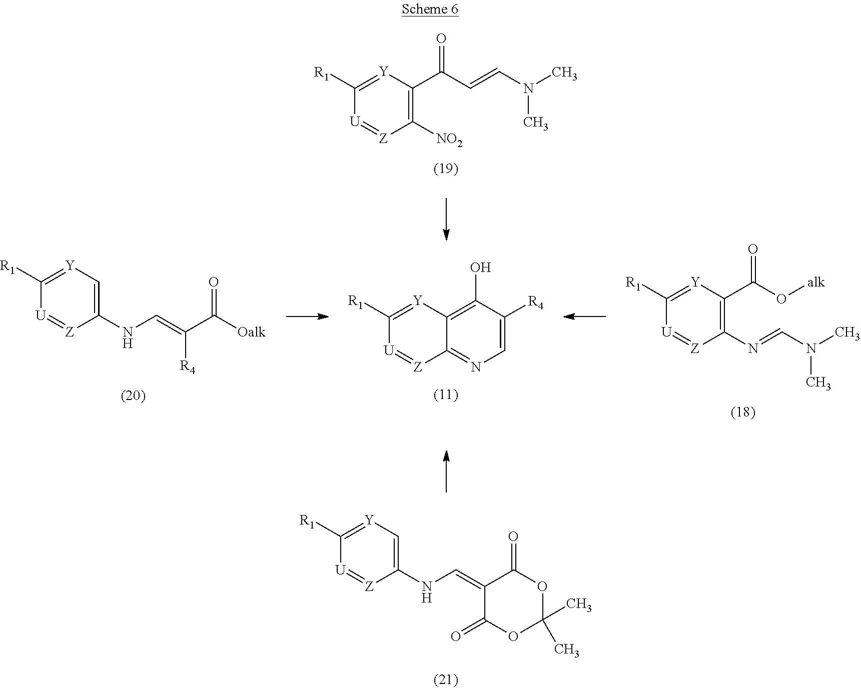

Compounds of formula (11) can be obtained by the reactions of compounds of formula (18), which can be synthesized according to literature; with a reagent, which is capable to replace dimethylamino group in formula (18) and subsequent to undergo cyclization by a nucleophilic attack on the ester group. Such reagent is, for example, acetonitrile, see for example: US2009/264427, EP1950201, WO2006/2047.

Starting from compounds of formula (19), which are either commercially available or can be synthesized according to literature, the nitro group can be reduced to the corresponding amino group, which can undergo cyclization to afford compounds of formula (11). For example, see WO2010/105761, European Journal of Medicinal Chemistry 2014, vol 87, p 508-518.

Compounds of formula (20) or compounds of formula (21), which can be synthesized according literature, undergo cyclization to afford compounds of formula (11) under elevated temperatures. For example, see: EP2566477, USUS2008/234267, EP3072893, WO201721319, WO2006/81182.

In accordance with a second aspect, the present invention covers methods of preparing compounds of general formula (I) as defined supra, said methods comprising the step of allowing an intermediate compound of general formula (2):

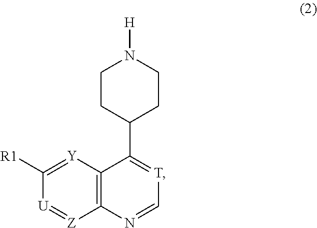

in which T, Y, R1, U, Z, and R3 are as defined for the compound of general formula (I) supra, to react with a compound of general formula (3)

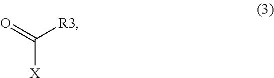

in which R3 is as defined for the compound of general formula (I) supra, and X is a leaving group such as a halogen atom, such as Br, C or I for example, a hydroxyl group, a C₁-C₆-alkyl-O— group, a C₁-C₆-alkyl-C(═O)—O— group, or an aryl-C(═O)—O— group,
optionally in the presence of a base, such as triethylamine, pyridine, N-ethyl-N,N-diisopropylamine, for example,
optionally in a solvent, such as an aprotic polar or a non-polar solvent such as acetonitrile, dichloromethane, 1,2 dichloroethane, chloroform, N,N-dimethylformamide (DMF), 1-methyl-pyrrolidin-2-one (NMP), or mixture of same,
optionally at ambient or an elevated temperature,
optionally in the presence of a catalyst, such as N,N-dimethylaminopyridine for example, thereby giving a compound of general formula (I)

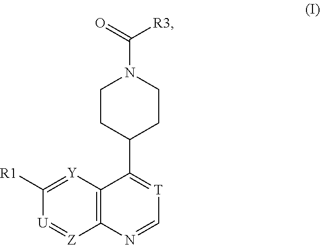

in which
T, Y, R1, U, Z, and R3 are as defined for the compound of general formula (I) supra,
then optionally converting said compound of general formula (I) into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

In accordance with a fourth aspect, the present invention covers intermediate compounds which are useful for the preparation of the compounds of general formula (I), supra.

Particularly, the inventions covers the intermediate compounds of general formula (2′)

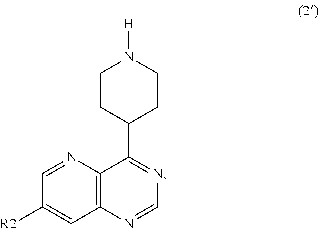

in which R2 is defined for the compound of general formula (I) supra.

In accordance with a fifth aspect, the present invention covers the use of said intermediate compounds for the preparation of a compound of general formula (I) as defined supra.

Particularly, the inventions covers the use of intermediate compounds of general formula (2′)

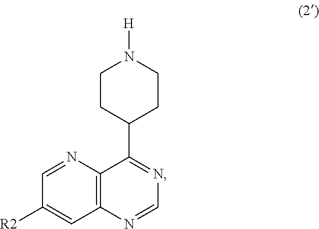

in which R2 is defined for the compound of general formula (I) supra, for the preparation of a compound of general formula (I) as defined supra.

The present invention covers the intermediate compounds which are disclosed in the Example Section of this text, infra.

The present invention covers any sub-combination within any embodiment or aspect of the present invention of intermediate compounds of general formula (2′) supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. Compounds of the present invention have surprisingly been found to effectively inhibit ERK5 and it is possible therefore that said compounds be used for the treatment or prophylaxis of diseases, preferably cancer disorders in humans and animals.

Compounds of the present invention can be utilized to inhibit, block, reduce, decrease, etc., cell proliferation and/or cell division, and/or produce apoptosis in tumors with ERK5 genomically amplified and/or with constitutively active ERK5 signalling. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Examples of breast cancers include, but are not limited to, invasive ductal carcinoma, invasive lobular carcinoma, ductal carcinoma in situ, and lobular carcinoma in situ.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

### Kidney Cancer.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In another aspect, the cell is in vitro. In another embodiment, the cell is in vivo.

The present invention also provides methods of treating cancer, in particular those disorders mentioned supra.

These disorders have been well characterized in humans, but also exist with a similar etiology in other mammals, and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of cancer, in particular those disorders mentioned supra.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

The pharmaceutical activity of the compounds according to the invention can be explained by their activity as ERK5 inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

In accordance with a further aspect, the present invention covers the use of a compound of formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of diseases, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

In accordance with a further aspect, the present invention covers a method of treatment or prophylaxis of diseases, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxyethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and block copolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

Particularly, the present invention covers a pharmaceutical combination, which comprises:
one or more first active ingredients, in particular compounds of general formula (I) as defined supra, and
one or more further active ingredients, in particular cancer, such as breast cancers, such as invasive ductal carcinoma, invasive lobular carcinoma, ductal carcinoma in situ, and lobular carcinoma in situ for example; liver cancers, such as hepatocellular carcinoma, cholangiocarcinoma, or mixed hepatocellular cholangiocarcinoma for example; or kidney cancers.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-cancer agents.

Examples of anti-cancer agents include:

131I-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, rucaparib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of cancer, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Experimental Section

NMR peak forms are stated as they appear in the spectra, possible higher order effects have not been considered.

TABLE 1
Abbreviations
The following table lists the abbreviations used herein.

| Abbreviation | Meaning |
|---|---|
| Ac₂O | acetic anhydride |
| AcOH | acetic acid (ethanoic acid) |
| aq. | aqueous |
| Boc | tert-butoxycarbonyl |
| BOP | (benzotriazol-1-yloxy)tris(dimethylamino) |
| | phosphonium hexafluorophosphate |
| br | broad (1H-NMR signal) |
| cat. | catalytic |
| conc. | concentrated |
| Cl | chemical ionisation |
| d | doublet |
| DAD | diode array detector |
| DBU | 1,8-diazabicyclo(5.4.0)undec-7-ene |
| DCC | N,N′-dicyclohexylcarbodiimide |
| DCM | dichloromethane |
| dd | double-doublet |
| DIC | N,N′-diisopropylcarbodiimide |
| DIPEA | diisopropylethylamine |
| DMA | N,N-dimethylacetamide |
| DMF | N,N-dimethylformamide |
| DMSO | dimethylsulfoxide |
| dt | double-triplet |
| EDC | 1-ethyl-3-(3-dimethylaminopropyl)carbodiimide |
| ELSD | Evaporative Light Scattering Detector |
| EtOAc | ethyl acetate |
| EtOH | ethanol |
| eq. | equivalent |
| ESI | electrospray (ES) ionisation |
| h | hour(s) |
| HATU | 1-[bis(dimethylamino)methylene]-1H-1,2,3-triazolo |
| | [4,5-b]pyridinium 3-oxid hexafluorophosphate |
| HBTU | (o-benzotriazole-10yl)-N,N,N′,N,- |
| | tetramethyluronium hexafluorophosphate |
| HCI | hydrochloric acid |
| HPLC | high performance liquid chromatography |
| LC-MS | liquid chromatography mass spectrometry |
| m | multiplet |
| min | minute(s) |
| MeCN | acetonitrile |
| MeOH | methanol |
| MS | mass spectrometry |
| NBS | N-bromosuccinimide |
| NCS | N-chlorosuccinimide |
| NMR | nuclear magnetic resonance spectroscopy: chemical |
| | shifts (δ) are given in ppm. The chemical shifts |
| | were corrected by setting the DMSO signal to 2.50 |
| | ppm unless otherwise stated. |
| PDA | Photo Diode Array |
| Pd/C | palladium on activated charcoal |
| PdCl₂(dppf) | [1,1′-bis(diphenylphosphino)ferrocene] |
| | dichloropalladium(II) |
| Pd(dba)₂ | bis(dibenzylideneacetone)palladium |
| PyBOP | (benzotriazol-1-yloxy)tripyrrolidinophosphonium |
| | hexafluorophosphate |
| a | quartet |
| r.t. or rt or RT | room temperature |
| rac | racemic |
| Rt | retention time (as measured either with HPLC or |
| | UPLC) in minutes |
| s | singlet |
| sat. | saturated |
| SIBX | stabilized 2-iodoxybenzoic acid |
| SM | starting material |
| SQD | Single-Quadrupole-Detector |
| t | triplet |
| T3P | propylphosphonic anhydride |
| TBAF | tetra-n-butylammonium fluoride |
| TBDMS | tert-butyldimethylsilyl |
| TBTU | N-[(1H-benzotriazol-1-yloxy)(dimethylamino) |
| | methylene]-N-methylmethanaminium |
| | tetrafluoroborate |
| td | triple-doublet |
| TEA | triethylamine |
| TFA | trifluoroacetic acid |
| THF | tetrahydrofuran |
| UPLC | ultra performance liquid chromatography |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary.

In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

All solvents used were commercially available and were used without further purification. Reactions were typically run using anhydrous solvents under an inert atmosphere of nitrogen.

Proton NMR spectra were recorded using a Bruker Plus 400 NMR Spectrometer unless stated otherwise. All deuterated solvents contained typically 0.03% to 0.05% v/v tetramethylsilane, which was used as the reference signal (set at δ 0.00 for both ¹H and ³C).

### UPLC-MS Standard Procedures

Analytical UPLC-MS was performed as described below. The masses (m/z) are reported from the positive mode electrospray ionisation unless the negative mode is indicated (ESI-). In most of the cases method 1 is used. If not, it is indicated.

### LC-MS Method 1

System: Shimadzu LC-MS: UFLC 20-AD and LCMS 2020 MS detector

Column: Shim-pack XR-ODS 2.2 μm, 3.0×50 mm

Solvent: A=H₂O+0.05% vol. HCOOC (99%)
B=acetonitrile+0.05% vol. HCOOC (99%)

### LC-MS Method 2

System: Shimadzu LC-MS: UFLC 20-AD and LCMS 2020 MS detector

Column: Shim-pack XR-ODS 2.2 μm, 3.0×50 mm

Solvent: A=H₂O+0.05% vol. TFA (99%)
B=acetonitrile+0.05% vol. TFA (99%)

### LC-MS Method 3

System: Shimadzu LC-MS: UFLC 20-AD and LCMS 2020 MS detector

Column: Shim-pack XR-ODS 2.2 μm, 3.0×50 mm

Solvent: A=H₂O+0.05% vol. NH₄HCO₃ (99%)
B=acetonitrile+0.05% vol. NH₄HCO₃ (99%)

### LC-MS Method 4:

System: Agilent 1290 UHPLC-MS Tof

Column: BEH C 18 (Waters) 1.7 μm, 50×2.1 mm

Solvent: A=H₂O+0.05% vol. HCOOC (99%)
B=acetonitrile+0.05% vol. HCOOC (99%)

Gradient: 0-1.7 min 2-90% B, 1.7-2 min 90% B, 2-2.5 min 90-2% B

Flow: 1.2 mL/min

Temperature: 60° C.

Detection: DAD scan range 210-400 nm

### LC-MS Method 5

Instrument MS: Waters ZQ; Instrument HPLC: Waters UPLC Acquity; Column: Acquity BEH C18 (Waters), 50 mm×2.1 mm, 1.7 μm; eluent A: water+0.1 vol % formic acid, eluent B: acetonitrile (Lichrosolv Merck); gradient: 0.0 min 99% A—1.6 min 1% A—1.8 min 1% A—1.81 min 99% A—2.0 min 99% A; temperature: 60° C.; flow: 0.8 ml/min; UV-Detection PDA 210-400 nm.

### EXAMPLES

### Example 1

### [4-(6,7-Dimethoxyquinazolin-4-yl)piperidin-1-yl][3-(trifluoromethoxy)phenyl]-methanone

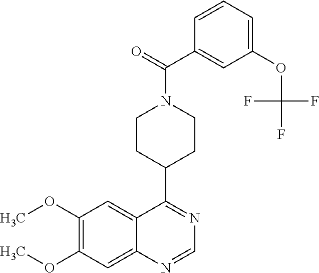 [SMILES: COc1cc2ncnc(C3CCN(C(=O)c4cccc(OC(F)(F)F)c4)CC3)c2cc1OC]

### Step 1: 6, 7-Dimethoxyquinazolin-4(3H)-one

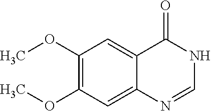

To a solution of 2-amino-4, 5-dimethoxybenzoic acid (5.0 g, 25.4 mmol) in 100 mL of 2-methoxyethanol was added formamidine acetate (4.0 g, 38.0 mmol). The resulting mixture was stirred at 100° C. for overnight. After cooled to room temperature, the solvent was removed in vacuo and the residue was diluted with 150 mL of ammonium hydroxide (10% water solution). The precipitated solid was collected by filtration and the filter cake was washed with water and dried in air to give 4.70 g (88%) of the title compound as a dark brown solid. MS (ESIpos): m/z=207 (M+H)⁺. LC-MS [Method 2]: Rₜ=1.12 min.

### Step 2: 4-Chloro-6,7-dimethoxyquinazoline

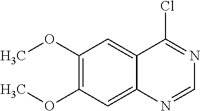

6,7-Dimethoxyquinazolin-4(3H)-one (20 g, 9.7 mmol) and 0.1 mL of N,N-dimethylformamide were added to 50 mL of thionyl chloride. The resulting mixture was stirred at reflux for overnight. After cooled to room temperature, the solvent was removed in vacuo and saturated sodium carbonate solution was added to adjust the pH value to 8 at 0° C. The resulting mixture was extracted with dichloromethane and the combined organic layer was dried over anhydrous sodium sulfate. The solvent was removed in vacuo and the residue was purified by silica gel column chromatography (petroleum ether:ethyl acetate=5:1) to give 1.96 g (88%) of the title compound as a yellow solid. MS (ESIpos): m/z=225 (M+H)⁺; LC-MS [Method 1]: Rₜ=0.91 min.

### Step 3: Tert-butyl 4-(6, 7-dimethoxyquinazolin-4-yl)-3, 6-dihydropyridine-1(2H)-carboxylate

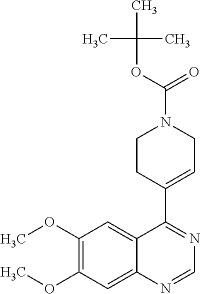 [SMILES: COc1cc2ncnc(C3=CCN(C(=O)OC(C)(C)C)CC3)c2cc1OC]

To a solution of 4-chloro-6,7-dimethoxyquinazoline (0.8 g, 3.6 mmol) in 10 mL of 1,4-dioxane/water (v:v=5:1) were added tert-butyl 4-(4,4,5,5-tetramethyl-1,3,2-dioxaborolan-2-yl)-5,6-dihydropyridine-1(2H)-carboxylate (1.70 g, 5.3 mmol), sodium carbonate (1.50 g, 14.2 mmol), and 1,1′-bis(diphenylphosphino)ferrocenepalladium(II) chloride (0.3 g, 0.4 mmol). The resulting mixture was stirred at 100° C. for 4 hours under nitrogen atmosphere. After cooled to room temperature, water was added and the resulting mixture was extracted with ethyl acetate. The combined organic layer was washed with water, brine, dried over anhydrous sodium sulfate and concentrated in vacuo. The residue was purified by chromatography to give 0.98 g (74%) of the title compound as a yellow solid. MS (ESIpos): m/z=372 (M+H)⁺. LC-MS [Method 3]: Rₜ=1.33 min. ¹H-NMR (400 MHz, DMSO-d₆): δ1.52 (s, 9H), 2.69-2.71 (m, 2H), 3.74-3.76 (m, 2H), 3.98 (s, 3H), 4.04 (s, 3H), 4.18-4.22 (m, 2H), 6.24 (s, 1H), 7.33 (s, 1H), 7.46 (s, 1H), 8.95 (s, 1H).

### Step 4: 6, 7-Dimethoxy-4-(1, 2, 3, 6-tetrahydropyridin-4-yl) quinazoline

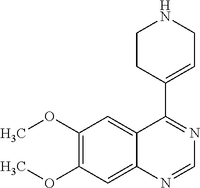 [SMILES: COc1cc2ncnc(C3=CCNCC3)c2cc1OC]

Tert-butyl 4-(6, 7-dimethoxyquinazolin-4-yl)-3, 6-dihydropyridine-1(2H)-carboxylate (0.8 g, 2.2 mmol), was dissolved in 3 mL of dichloromethane. Trifluoroacetic acid (3 mL, 39 mmol) was added and the resulting mixture was stirred at room temperature for 2 h. After evaporation in vacuo, saturated aqueous sodium carbonate was added to adjust the pH=8. The mixture was extracted with dichloromethane and the combined organic phase was dried over anhydrous sodium sulfate. After removal of the solvent, 0.51 g (87%) of the product was obtained as a white solid. ¹H-NMR (400 MHz, CD₃OD): δ 2.60-2.64 (m, 2H), 3.15 (t, 2H), 3.59 (t, 2H), 3.98 (s, 3H), 4.03 (s, 3H), 6.19 (s, 1H), 7.29 (s, 1H), 7.52 (s, 1H), 8.93 (s, 1H). MS (ESIpos): m/z=272 (M+H)⁺. LC-MS [Method 2]: Rₜ=0.79 min. ¹H-NMR (400 MHz, CD₃OD): δ 2.60-2.64 (m, 2H), 3.15 (t, 2H), 3.59 (t, 2H), 3.98 (s, 3H), 4.03 (s, 3H), 6.19 (s, 1H), 7.29 (s, 1H), 7.52 (s, 1H), 8.93 (s, 1H).

### Step 5: 6, 7-Dimethoxy-4-(piperidin-4-yl) quinazoline

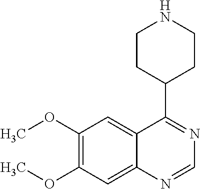 [SMILES: COc1cc2ncnc(C3CCNCC3)c2cc1OC]

6, 7-Dimethoxy-4-(1, 2, 3, 6-tetrahydropyridin-4-yl) quinazoline (310 mg, 1.1 mmol), was dissolved in 20 mL of MeOH. After addition of 0.1 g of palladium/carbon The resulting mixture was stirred at room temperature for overnight under a hydrogen atmosphere (3 atm). The solid was removed by filtration and the filtrate was concentrated in vacuo to give 256.0 mg (80%) of the product as a white solid.

MS (ESIpos): m/z=274 (M+H)⁺. LC-MS [Method 1]: Rₜ=0.67 min.

### Step 6: [4-(6,7-Dimethoxyquinazolin-4-yl)piperidin-1-yl][3-(trifluoromethoxy)-phenyl]methanone

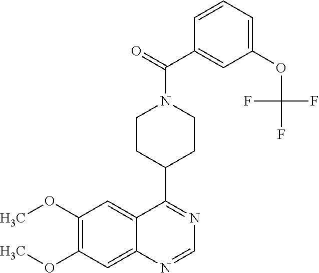 [SMILES: COc1cc2ncnc(C3CCN(C(=O)c4cccc(OC(F)(F)F)c4)CC3)c2cc1OC]

To a mixture of 6,7-dimethoxy-4-(piperidin-4-yl)quinazoline (80 mg, 0.3 mmol), N,N-diisopropylethylamine (114 mg, 0.9 mmol), and N,N,N′,N′-tetramethyl-O-(7-azabenzotriazol-1-yl)uronium hexafluorophosphate (167 mg, 0.5 mmol) in 2 mL of N,N-dimethylformamide, was added 3-(trifluoromethoxy)benzoic acid, 78 mg (0.4 mmol). The resulting mixture was stirred at room temperature for 2 hours. Water was added and the resulting solution was extracted with ethyl acetate. The combined organic layer was dried over anhydrous sodium sulfate and the solvent as removed in vacuo. The residue was purified by chromatography to give 15.8 mg (12%) of the product as a white solid. MS (ESIpos): m/z=462 (M+H)⁺; LC-MS [Method 2]: Rₜ=1.34 min. ¹H-NMR (300 MHz, DMSO-d₆): δ 1.86-1.93 (m, 4H), 3.05-3.23 (m, 1H), 3.44-3.52 (m, 2H), 3.55-3.69 (m, 1H), 3.97 (s, 3H), 4.00 (s, 3H), 4.64-4.71 (m, 1H), 7.35 (s, 1H), 7.40-7.50 (m, 3H), 7.57-7.73 (m, 2H), 9.01 (s, 1H).

### Example 2

### [4-(6,7-Dimethoxyquinazolin-4-yl)piperidin-1-yl][2-fluoro-4-(trifluoromethoxy)phenyl]methanone

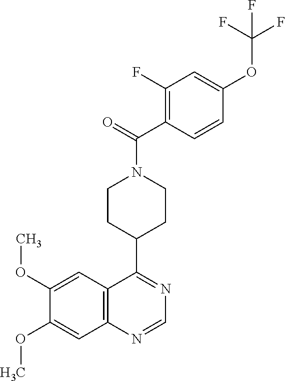 [SMILES: COc1cc2ncnc(C3CCN(C(=O)c4ccc(OC(F)(F)F)cc4F)CC3)c2cc1OC]

A solution of 6,7-dimethoxy-4-(piperidin)-4-yl)quinazoline (80 mg, 0.293 mmol,), 2-fluoro-4-(trifluoromethoxy)benzoyl chloride (99 mg, 0.41 mmol, and triethylamine (123 μl, 0.88 mmol) in dichloromethane (2.5 ml) was stirred at ambient temperature overnight. The solvent was evaporated and the residue was purified by chromatography The title compound was obtained in 45% yield (66 mg). ¹H NMR (400 MHz, DMSO-d₆) δ9.00 (s, 1H), 7.63 (s, 1H), 7.52-7.58 (m, 2H), 7.32-7.39 (m, 2H), 4.67 (br d, J=13.43 Hz, 1H), 3.99 (s, 3H), 3.97 (s, 3H), 3.48-3.59 (m, 1H), 3.37-3.46 (m, 1H), 3.07-3.18 (m, 1H), 1.94-2.02 (m, 1H), 1.77-1.91 (m, 3H). LCMS (Method 4): Rₜ=1.13 min; MS (ESIpos): m/z=480.1 [M+H]⁺.

### Example 3

### 4-{[4-(6,7-Dimethoxyquinazolin-4-yl)piperidin-1-yl]carbonyl}benzonitrile

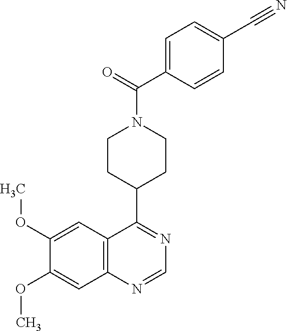 [SMILES: COc1cc2ncnc(C3CCN(C(=O)c4ccc(C#N)cc4)CC3)c2cc1OC]

To 4-cyanobenzoic acid (29 mg, 195 μmol), a solution of 6,7-dimethoxy-4-(piperidin-4-yl)-quinazoline (41.0 mg, 150 μmol) in 1 mL NMP was added. HATU (74.1 mg, 195 μmol) in 0.5 mL NMP and N,N-diisopropylethylamine (75.6 mg, 585 μmol) in 0.5 mL NMP were added. The reaction mixture was shaked for 24 hours at room temperature. Precipitated material was filtered off and the filtrate was purified by preparative HPLC to give the title compound (6 mg, 10% yield). LC-MS [Method 5]: Rₜ=0.94 min; MS (ESIpos): m/z=403 [M+H]⁺.

### Example 4

### 4-[1-(4-Chlorobenzoyl)piperidin-4-yl]-6,7-dimethoxyquinazoline

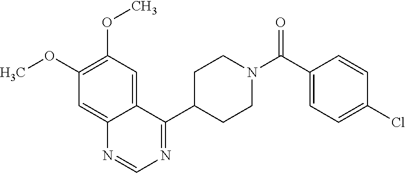 [SMILES: COc1cc2ncnc(C3CCN(C(=O)c4ccc(Cl)cc4)CC3)c2cc1OC]

This compound was synthesized by the same method as described in example 3 to afford the desired product in 33% yields (33 mg).

MS (ESIpos): m/z=412 [M+H]⁺.

### Example 5

### [4-(6,7-Dimethoxyquinazolin-4-yl)piperidin-1-yl]{4-[(trifluoromethoxy)methyl]phenyl}methanone

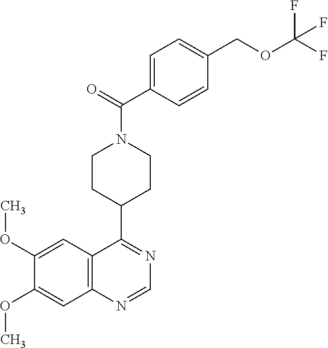 [SMILES: COc1cc2ncnc(C3CCN(C(=O)c4ccc(COC(F)(F)F)cc4)CC3)c2cc1OC]

This compound was synthesized by the same method as described in example 3 to afford the desired product. LC-MS [Method 5]: Rₜ=1.16 min; MS (ESIpos): m/z=476 [M+H]⁺

### Example 6

### 4-[1-(4-Fluorobenzoyl)piperidin-4-yl]-6,7-dimethoxyquinazoline

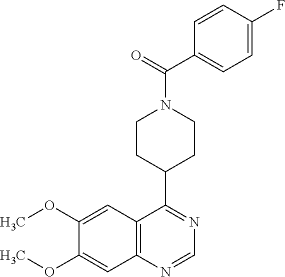 [SMILES: COc1cc2ncnc(C3CCN(C(=O)c4ccc(F)cc4)CC3)c2cc1OC]

To a solution of 6,7-dimethoxy-4-(piperidin-4-yl) quinazoline (100 mg, 0.37 mmol), 4-dimethylaminopyridine (2 mg, 0.016 mmol) and triethylamine (0.26 mL, 1.84 mmol) in 1,2-dichloroethane was added 4-fluorobenzoyl chloride (0.05 mL, 0.44 mmol). The reaction mixture was stirred 20 minutes at room temperature, followed by 1 h at 55° C. and for further 16 h at room temperature. The reaction was diluted with methanol. After removal of the solvent and chromatography the desired product was obtained in 38% yields (55 mg).

MS (ESIpos): m/z=396 [M+H]⁺.

### Example 7

### 6,7-Dimethoxy-4-{1-[4-(trifluoromethoxy)benzoyl]piperidin-4-yl}quinazoline

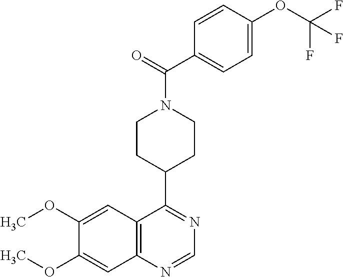 [SMILES: COc1cc2ncnc(C3CCN(C(=O)c4ccc(OC(F)(F)F)cc4)CC3)c2cc1OC]

This compound was synthesized by the same method as described in example 1 to afford the desired product in 18% yields

MS(ESIpos): m/z=462 (M+H)⁺.

### Example 8

### 6,7-Dimethoxy-4-{1-[4-(trifluoromethyl)benzoyl]piperidin-4-yl}quinazoline

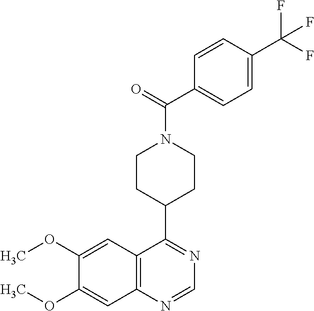 [SMILES: COc1cc2ncnc(C3CCN(C(=O)c4ccc(C(F)(F)F)cc4)CC3)c2cc1OC]

This compound was synthesized by the same method as described in example 6 to afford the desired product in 61% yields.

¹H NMR (400 MHz, DMSO-d₆) δ9.02 (s, 1H), 7.84 (d, J=7.86 Hz, 2H), 7.67 (d, J=7.86 Hz, 2H), 7.58 (s, 1H), 7.35 (s, 1H), 4.65 (s, 1H), 3.94-4.03 (m, 7H), 3.56-3.69 (m, 1H), 3.35-3.45 (m, 1H), 3.00-3.23 (m, 1H), 1.73-2.10 (m, 4H). LC-MS [Method 4]: Rₜ=1.07 min; MS (ESIpos): m/z=446.1 [M+H]⁺.

### Example 9

### [4-(6,7-Dimethoxyquinazolin-4-yl)piperidin-1-yl](phenyl)methanone

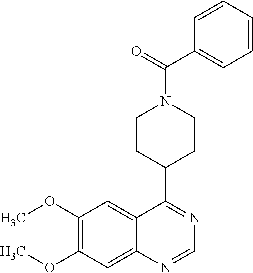 [SMILES: COc1cc2ncnc(C3CCN(C(=O)c4ccccc4)CC3)c2cc1OC]

6,7-Dimethoxy-4-(piperidin-4-yl)quinazoline (80 mg, 0.3 mmol), and benzoyl chloride, (53 mg, 0.4 mmol) were dissolved in 2 mL of dichloromethane at room temperature. Then 0.08 mL of triethylamine was added. The reaction mixture was stirred at room temperature for 2 hours. The reaction mixture was concentrated under vacuum, the residue was purified by chromatography to give of the product as a yellow solid (35.3 mg 31%).

¹H-NMR (300 MHz, DMSO-d₆): δ1.85-1.88 (m, 4H), 2.92-3.16 (m, 1H), 3.36-3.58 (m, 2H), 3.64-3.82 (m, 1H), 3.97 (s, 3H), 4.00 (s, 3H), 4.68-4.69 (m, 1H), 7.35 (s, 1H), 7.43-7.48 (m, 5H), 7.57 (s, 1H), 9.01 (s, 1H). MS (ESIpos): m/z=378 (M+H)⁺.

### Example 10

### [4-(6,7-Dimethoxyquinazolin-4-yl)piperidin-1-yl][4-(propan-2-yloxy)phenyl] methanone

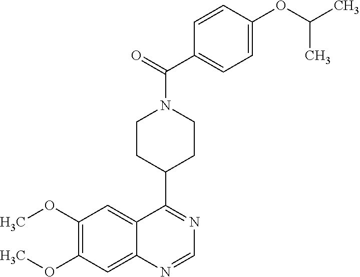 [SMILES: COc1cc2ncnc(C3CCN(C(=O)c4ccc(OC(C)C)cc4)CC3)c2cc1OC]

This compound was synthesized by the same method as described in example 1 to give 15.2 mg (9%) of the title compound as a yellow solid. MS (ESIpos): m/z=436 (M+H)⁺; LC-MS [Method 2]: Rₜ=1.27 min. ¹H-NMR (400 MHz, DMSO-d₆): δ [ppm]=1.28 (d, 6H), 1.81-1.87 (m, 4H), 3.12-3.34 (m, 4H), 3.95-4.00 (m, 7H), 4.64-4.71 (m, 1H), 6.96 (d, 2H), 7.35-7.39 (m, 3H), 7.81 (s, 1H), 9.01 (s, 1H).

### Example 11

### [4-(6,7-Dimethoxyquinazolin-4-yl)piperidin-1-yl](4-methoxyphenyl)methanone

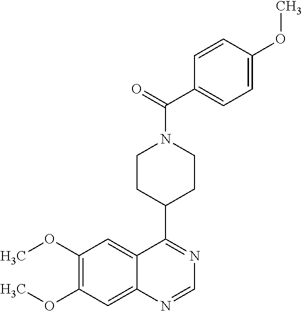

To a solution of 6,7-dimethoxy-4-(piperidin-4-yl)quinazoline (60 mg, 0.22 mmol,), triethylamine (92 μl, 0.66 mmol) in dichloromethane (2 ml) was added 4-methoxybenzoyl chloride (45 mg, 0.26 mmol) and the reaction was stirred at ambient temperature overnight. The solvent was removed under reduced pressure and the residue was purified by HPLC chromatography to obtain the title compound in 32% yield (30 mg). ¹H NMR (400 MHz, DMSO-d6) δ 9.00 (s, 1H), 7.57 (s, 1H), 7.39-7.43 (m, 2H), 7.35 (s, 1H), 7.03-7.05 (m, 1H), 6.98-7.03 (m, 2H), 4.00 (s, 3H), 3.97 (s, 3H), 3.80 (s, 3H), 1.81-1.92 (m, 4H). LC-MS [Method 4]: Rₜ=0.91 min; MS (ESIpos): m/z=408.2 [M+H]⁺.

### Example 12

### (4-Bromophenyl)[4-(6,7-dimethoxyquinazolin-4-yl)piperidin-1-yl]methanone

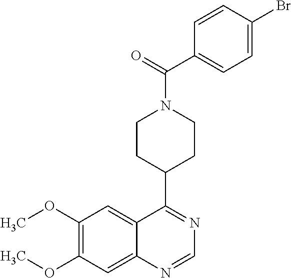 [SMILES: COc1cc2ncnc(C3CCN(C(=O)c4ccc(Br)cc4)CC3)c2cc1OC]

This compound was synthesized by the same method as described in example 9 to give the title compound in 28% yield (29 mg). ¹H NMR (400 MHz, DMSO-d₆) δ9.00 (s, 1H), 7.67 (d, J=8.36 Hz, 2H), 7.57 (s, 1H), 7.41 (d, J=8.36 Hz, 2H), 7.35 (s, 1H), 4.54-4.71 (m, 1H), 3.99 (s, 3H), 3.99-3.99 (m, 1H), 3.97 (s, 3H), 3.64-3.77 (m, 1H), 3.34-3.44 (m, 1H), 3.04-3.20 (m, 1H), 1.76-2.03 (m, 4H). LCMS LC-MS [Method 4]: Rₜ=1.05 min; MS (ESIpos): m/z=456.1 & 458.1 [M+H]⁺.

### Example 13

### [4-(6,7-Dimethoxyquinazolin-4-yl)piperidin-1-yl](4-methylphenyl)methanone

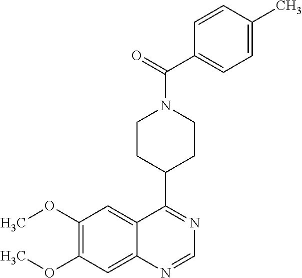

This compound was synthesized by the same method as described in example 9 to give the title compound in 33% yield (30 mg). ¹H NMR (400 MHz, DMSO-d₆) δ9.00 (s, 1H), 7.57 (s, 1H), 7.32-7.36 (m, 3H), 7.25-7.29 (m, 2H), 4.56-4.69 (m, 1H), 3.99 (s, 3H), 3.97 (s, 3H), 3.73-3.85 (m, 1H), 3.04-3.18 (m, 1H), 2.34 (s, 3H), 1.79-1.95 (m, 4H). LC-MS [Method 4]: Rₜ=1.03 min; MS (ESIpos): m/z=392.2 [M+H]⁺.

### Example 14

### (4-Tert-butylphenyl)[4-(6,7-dimethoxyquinazolin-4-yl)piperidin-1-yl]methanone

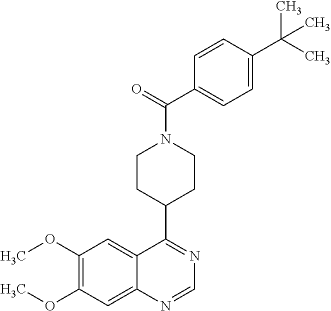 [SMILES: COc1cc2ncnc(C3CCN(C(=O)c4ccc(C(C)(C)C)cc4)CC3)c2cc1OC]

This compound was synthesized by the same method as described in example 9 to give the title compound in 40% yield (40 mg). ¹H NMR (400 MHz, DMSO-d₆) δ9.01 (s, 1H), 7.57 (s, 1H), 7.46-7.50 (m, 2H), 7.34-7.39 (m, 3H), 4.57-4.71 (m, 1H), 3.99 (s, 3H), 3.97 (s, 3H), 3.76-3.86 (m, 1H), 3.34-3.41 (m, 1H), 3.04-3.18 (m, 1H), 1.74-2.05 (m, 4H), 1.30 (s, 9H). LC-MS [Method 4]: Rₜ=1.21 min; MS (ESIpos): m/z=434.2 [M+H]⁺.

### Example 15

### [4-(6,7-Dimethoxyquinazolin-4-yl)piperidin-1-yl](4-ethoxyphenyl)methanone

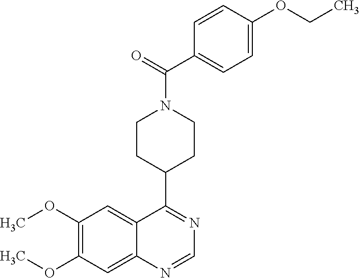 [SMILES: CCOc1ccc(C(=O)N2CCC(c3ncnc4cc(OC)c(OC)cc34)CC2)cc1]

This compound was synthesized by the same method as described in example 9 to give the title compound in 61% yield (59 mg). ¹H NMR (400 MHz, DMSO-d₆) δ9.00 (s, 1H), 7.57 (s, 1H), 7.39 (d, J=8.62 Hz, 2H), 7.35 (s, 1H), 6.98 (d, J=8.87 Hz, 2H), 4.45 (br d, J=13.94 Hz, 1H), 4.06 (q, J=6.84 Hz, 2H), 4.00 (s, 3H), 3.97 (s, 3H), 3.03-3.29 (m, 2H), 1.76-2.06 (m, 4H), 1.34 (t, J=6.97 Hz, 3H). LC-MS [Method 4]: Rₜ=1.04 min; MS (ESIpos): m/z=422.2 [M+H]⁺.

### Example 16

### [4-(6,7-Dimethoxyquinazolin-4-yl)piperidin-1-yl]{4-[(trifluoromethyl)sulfanyl]-phenyl}methanone

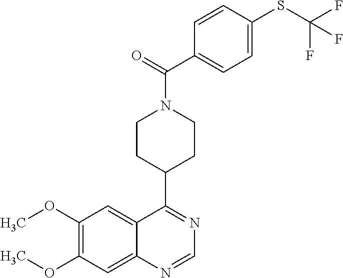 [SMILES: COc1cc2ncnc(C3CCN(C(=O)c4ccc(SC(F)(F)F)cc4)CC3)c2cc1OC]

This compound was synthesized by the same method as described in example 9 to give the title compound in 37% yield (55 mg). ¹H NMR (400 MHz, DMSO-d₆) δ9.01 (s, 1H), 7.81 (d, J=8.11 Hz, 2H), 7.55-7.63 (m, 3H), 7.35 (s, 1H), 4.54-4.71 (m, 1H), 3.98 (d, J=10.14 Hz, 7H), 3.58-3.71 (m, 1H), 3.37-3.46 (m, 1H), 3.12 (br s, 1H), 1.78-2.05 (m, 4H). LC-MS [Method 4]: Rₜ=1.20 min; MS (ESIpos): m/z=478.1 [M+H]⁺.

### Example 17

### (4-Butoxyphenyl)[4-(6,7-dimethoxyquinazolin-4-yl)piperidin-1-yl]methanone

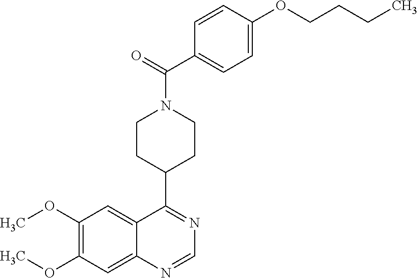 [SMILES: CCCCOc1ccc(C(=O)N2CCC(c3ncnc4cc(OC)c(OC)cc34)CC2)cc1]

This compound was synthesized by the same method as described in example 9 to give the title compound in 35% yield (48 mg). ¹H NMR (400 MHz, DMSO-d₆) δ9.00 (s, 1H), 7.57 (s, 1H), 7.39 (d, J=8.62 Hz, 2H), 7.35 (s, 1H), 6.98 (d, J=8.87 Hz, 2H), 4.40-4.64 (m, 1H), 3.94-4.05 (m, 9H), 3.08-3.28 (m, 1H), 1.80-1.96 (m, 4H), 1.64-1.76 (m, 2H), 1.38-1.50 (m, 2H), 0.93 (t, J=7.48 Hz, 3H). LC-MS [Method 4]: Rₜ=1.20 min; MS (ESIpos): m/z=450.2 [M+H]⁺.

### Example 18

### [4-(6,7-Dimethoxyquinazolin-4-yl)piperidin-1-yl][4-(propan-2-yl)phenyl]-methanone

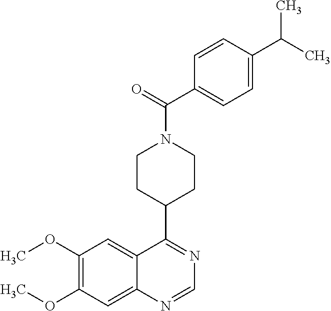 [SMILES: COc1cc2ncnc(C3CCN(C(=O)c4ccc(C(C)C)cc4)CC3)c2cc1OC]

This compound was synthesized by the same method as described in example 9 to give the title compound in 36% yield (46 mg). ¹H NMR (400 MHz, DMSO-d₆) δ9.01 (s, 1H), 7.57 (s, 1H), 7.30-7.39 (m, 5H), 4.56-4.76 (m, 1H), 3.99 (s, 3H), 3.97 (s, 3H), 3.73-3.88 (m, 1H), 3.01-3.19 (m, 1H), 2.93 (quin, J=6.84 Hz, 1H), 1.70-2.04 (m, 4H), 1.22 (d, J=6.84 Hz, 6H). LC-MS [Method 4]: Rₜ=1.15 min; MS (ESIpos): m/z=420.2 [M+H]⁺.

### Example 19

### [4-(Difluoromethoxy)phenyl][4-(6,7-dimethoxyquinazolin-4-yl)piperidin-1-yl]methanone

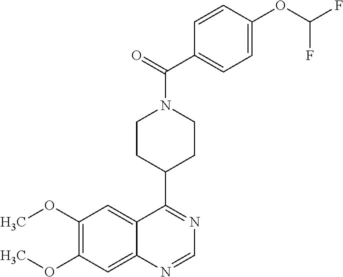 [SMILES: COc1cc2ncnc(C3CCN(C(=O)c4ccc(OC(F)F)cc4)CC3)c2cc1OC]

This compound was synthesized by the same method as described in example 9 to give the title compound in 27% yield (37 mg). ¹H NMR (400 MHz, DMSO-d₆) δ9.00 (s, 1H), 7.57 (s, 1H), 7.50-7.54 (m, 2H), 7.10-7.37 (m, 4H), 4.52-4.70 (m, 1H), 4.00 (s, 3H), 3.97 (s, 3H), 3.70-3.83 (m, 1H), 3.34-3.42 (m, 1H), 3.04-3.17 (m, 1H), 1.77-2.03 (m, 4H). LC-MS [Method 4]: Rₜ=0.98 min; MS (ESIpos): m/z=444.2 [M+H]⁺.

### Example 20

### [4-(6,7-Dimethoxyquinazolin-4-yl)piperidin-1-yl](4-propylphenyl)methanone

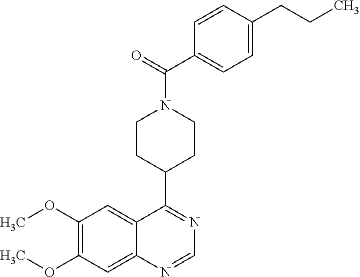 [SMILES: CCCc1ccc(C(=O)N2CCC(c3ncnc4cc(OC)c(OC)cc34)CC2)cc1]

This compound was synthesized by the same method as described in example 9 to give the title compound in 46% yield (59 mg). ¹H NMR (400 MHz, DMSO-d₆) δ9.00 (s, 1H), 7.57 (s, 1H), 7.32-7.39 (m, 3H), 7.24-7.29 (m, 2H), 4.51-4.72 (m, 1H), 3.99 (s, 3H), 3.97 (s, 3H), 3.80 (br d, J=10.39 Hz, 1H), 3.02-3.15 (m, 1H), 2.55-2.62 (m, 2H), 1.78-2.02 (m, 4H), 1.61 (sxt, J=7.45 Hz, 2H), 0.90 (t, J=7.35 Hz, 3H). LC-MS [Method 4]: Rₜ=1.17 min; MS (ESIpos): m/z=420.2 [M+H]⁺.

### Example 21

### [4-(6,7-Dimethoxyquinazolin-4-yl)piperidin-1-yl]{4-[(2,2,2-trifluoroethoxy)methyl]phenyl}-methanone

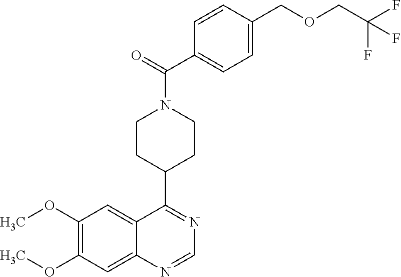 [SMILES: COc1cc2ncnc(C3CCN(C(=O)c4ccc(COCC(F)(F)F)cc4)CC3)c2cc1OC]

This compound was synthesized by the same method as described in example 9 to give the title compound in 41% yield (62 mg). ¹H NMR (400 MHz, DMSO-d6) δ9.01 (s, 1H), 7.57 (s, 1H), 7.40-7.48 (m, 4H), 7.35 (s, 1H), 4.71 (s, 2H), 4.59-4.67 (m, 1H), 4.14 (q, J=9.38 Hz, 2H), 3.99 (s, 3H), 3.97 (s, 3H), 3.66-3.81 (m, 1H), 3.34-3.40 (m, 1H), 3.06-3.17 (m, 1H), 1.75-2.05 (m, 4H). LC-MS [Method 4]: Rₜ=1.06 min; MS (ESIpos): m/z=490.2 [M+H]⁺.

### Example 22

### [4-(6,7-Dimethoxyquinazolin-4-yl)piperidin-1-yl][3-fluoro-4-(trifluoromethoxy)phenyl]methanone

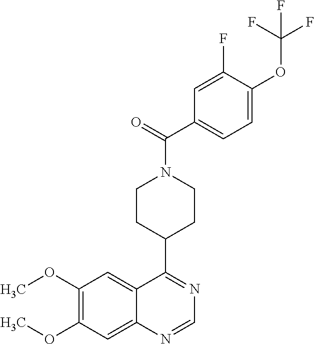 [SMILES: COc1cc2ncnc(C3CCN(C(=O)c4ccc(OC(F)(F)F)c(F)c4)CC3)c2cc1OC]

This compound was synthesized by the same method as described in example 9 to give the title compound in 47% yield (70 mg). ¹H NMR (400 MHz, DMSO-d6) δ9.00 (s, 1H), 7.63-7.71 (m, 2H), 7.57 (s, 1H), 7.41 (td, J=1.11, 8.17 Hz, 1H), 7.35 (s, 1H), 4.57-4.70 (m, 1H), 4.00 (s, 3H), 3.97 (s, 3H), 3.62-3.72 (m, 1H), 3.36-3.44 (m, 1H), 3.06-3.17 (m, 1H), 1.79-2.02 (m, 4H). LC-MS [Method 4]: Rₜ=1.14 min; MS (ESIpos): m/z=480.1 [M+H]⁺.

### Example 23

### [4-(6,7-Dimethoxyquinazolin-4-yl)piperidin-1-yl][6-(trifluoromethoxy)pyridin-3-yl]methanone

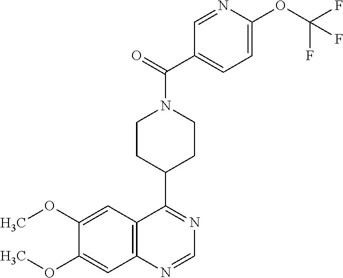 [SMILES: COc1cc2ncnc(C3CCN(C(=O)c4ccc(OC(F)(F)F)nc4)CC3)c2cc1OC]

To a solution of 6,7-dimethoxy-4-(piperidin)-4-yl)quinazoline (119 mg, 0.44 mmol, and 6-(trifluoromethoxy)nicotinic acid (99 mg, 0.48 mmol) in DMF (3.53 ml) was added 1-(3-dimethylaminopropyl)-3-ethylcarbodiimide hydrochloride (108.5 mg, 0.57 mmol) and N,N-diisopropylethylamine (0.227 ml). The reaction was stirred at ambient temperature overnight. The solvent was removed under reduced pressure and the residue was purified by chromatography to yield the title compound in 31% yield (65 mg). ¹H NMR (400 MHz, DMSO-d6) δ9.00 (s, 1H), 8.49 (d, J=2.28 Hz, 1H), 8.14 (dd, J=2.28, 8.36 Hz, 1H), 7.57 (s, 1H), 7.33-7.40 (m, 2H), 4.60-4.73 (m, 1H), 4.00 (s, 3H), 3.97 (s, 3H), 3.66-3.76 (m, 1H), 3.41-3.51 (m, 1H), 3.08-3.22 (m, 1H), 1.81-2.04 (m, 4H). LC-MS [Method 4]: Rₜ=1.01 min; MS (ESIpos): m/z=463.2 [M+H]⁺.

### Example 24

### [4-(6,7-Dimethoxyquinazolin-4-yl)piperidin-1-yl][4-(2-methoxyethoxy)phenyl]-methanone

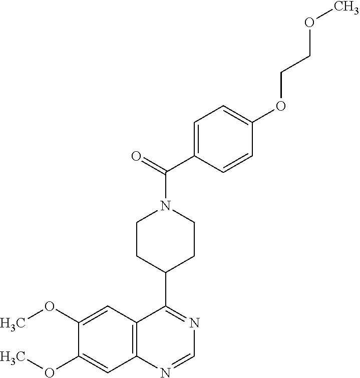

This compound was synthesized by the same method as described in example 1 to give the title compound in 36% yield (65 mg). ¹H NMR (400 MHz, DMSO-d6) δ 9.00 (s, 1H), 7.57 (s, 1H), 7.39 (d, J=8.87 Hz, 2H), 7.35 (s, 1H), 7.00 (d, J=8.87 Hz, 2H), 4.39-4.66 (m, 1H), 4.10-4.20 (m, 2H), 4.00 (s, 3H), 3.97 (s, 3H), 3.63-3.71 (m, 2H), 3.31 (s, 3H), 3.14-3.26 (m, 1H), 1.77-1.94 (m, 4H). LC-MS [Method 4]: Rₜ=0.90 min; MS (ESIpos): m/z=452.2 [M+H]⁺.

### Example 25

### (4-Tert-butoxyphenyl)[4-(6,7-dimethoxyquinazolin-4-yl)piperidin-1-yl]methanone

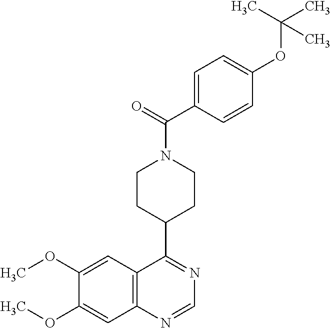 [SMILES: COc1cc2ncnc(C3CCN(C(=O)c4ccc(OC(C)(C)C)cc4)CC3)c2cc1OC]

This compound was synthesized by the same method as described in example 1 to give the title compound in 25% yield (46 mg). ¹H NMR (400 MHz, DMSO-d6) δ9.00 (s, 1H), 7.57 (s, 1H), 7.31-7.40 (m, 3H), 7.04 (d, J=8.62 Hz, 2H), 4.49-4.68 (m, 1H), 4.00 (s, 3H), 3.97 (s, 3H), 3.08-3.25 (m, 1H), 1.77-1.99 (m, 4H), 1.33 (s, 9H). LC-MS [Method 4]: Rₜ=1.10 min; MS (ESIpos): m/z=450.3 [M+H]⁺.

### Example 26

### [4-(6,7-Dimethoxyquinazolin-4-yl)piperidin-1-yl][5-(trifluoromethoxy)pyridin-2-yl]methanone

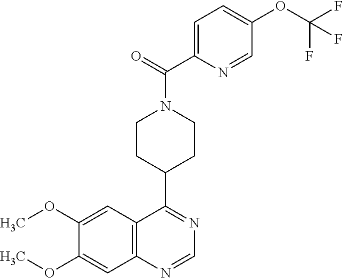 [SMILES: COc1cc2ncnc(C3CCN(C(=O)c4ccc(OC(F)(F)F)cn4)CC3)c2cc1OC]

This compound was synthesized by the same method as described in example 9 to give the title compound in 7% yields (16.8 mg). ¹H NMR (400 MHz, DMSO-d₆) δ9.00 (s, 1H), 8.61-8.77 (m, 1H), 8.05 (ddd, J=1.14, 2.66, 8.62 Hz, 1H), 7.79 (d, J=8.62 Hz, 1H), 7.58 (s, 1H), 7.35 (s, 1H), 4.66 (br d, J=13.18 Hz, 1H), 3.98 (d, J=10.90 Hz, 7H), 3.76 (br d, J=13.43 Hz, 1H), 3.35-3.42 (m, 1H), 3.14 (dt, J=2.79, 12.67 Hz, 1H), 1.71-2.05 (m, 4H). MS (ESIpos): m/z=463.5 [M+H]⁺.

### Example 27

### [4-(6,7-Dimethoxyquinazolin-4-yl)piperidin-1-yl][4-(prop-2-en-1-yloxy)phenyl]-methanone

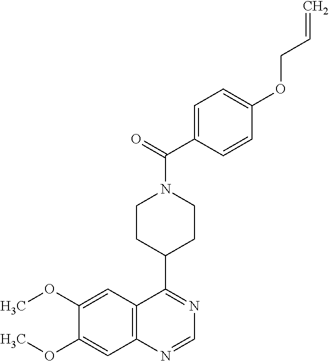 [SMILES: C=CCOc1ccc(C(=O)N2CCC(c3ncnc4cc(OC)c(OC)cc34)CC2)cc1]

This compound was synthesized by the same method as described in example 1 to give the title compound in 18% yield (32 mg). ¹H NMR (400 MHz, DMSO-d₆) δ9.00 (s, 1H), 7.57 (s, 1H), 7.40 (d, J=8.87 Hz, 2H), 7.35 (s, 1H), 7.01 (d, J=8.87 Hz, 2H), 5.98-6.15 (m, 1H), 5.41 (dd, J=1.77, 17.24 Hz, 1H), 5.27 (dd, J=1.65, 10.52 Hz, 1H), 4.61 (td, J=1.52, 5.32 Hz, 2H), 4.00 (s, 3H), 3.97 (s, 3H), 3.11-3.27 (m, 1H), 1.78-1.94 (m, 4H). LC-MS [Method 4]: Rₜ=1.03 min; MS (ESIpos): m/z=434.2 [M+H]⁺.

### Example 28

### [4-(6,7-Dimethoxyquinazolin-4-yl)piperidin-1-yl][2-(trifluoromethoxy)phenyl]-methanone

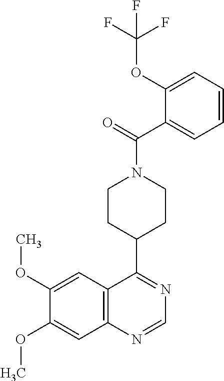 [SMILES: COc1cc2ncnc(C3CCN(C(=O)c4ccccc4OC(F)(F)F)CC3)c2cc1OC]

This compound was synthesized by the same method as described in example 3 to afford the desired product. LC-MS [Method 5]: Rₜ=1.12 min; MS (ESIpos): m/z=462 [M+H]⁺

### Example 29

### [4-(7-Methylquinazolin-4-yl)piperidin-1-yl][4-(trifluoromethoxy)phenyl]-methanone

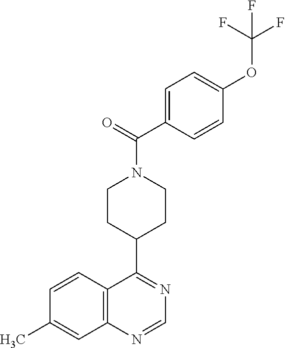 [SMILES: Cc1ccc2c(C3CCN(C(=O)c4ccc(OC(F)(F)F)cc4)CC3)ncnc2c1]

### Step 1: 7-Methylquinazolin-4(3H)-one

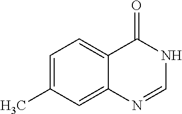

This compound was synthesized by the same method as described in example 1 to give 2.60 g (81%) of the product as a brown solid. MS (ESIpos): m/z=161 (M+H)⁺, LC-MS [Method 2]: Rₜ=0.61 min.

### Step 2: 4-Chloro-7-methylquinazoline

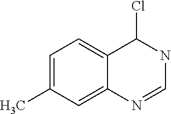

This compound was synthesized by the same method as described in example 1 to give 3.00 g (73%) of the product as a yellow solid. MS (ESIpos): m/z=179 (M+H)⁺, LC-MS [Method 2]: Rₜ=0.95 min.

### Step 3: Tert-butyl 4-(7-methylquinazolin-4-yl)-3,6-dihydropyridine-1(2H)-carboxylate

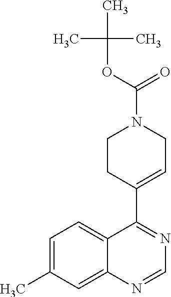 [SMILES: Cc1ccc2c(C3=CCN(C(=O)OC(C)(C)C)CC3)ncnc2c1]

This compound was synthesized by the same method as described in example 1 to give 96.2 mg (80%) of the product as a colorless oil. MS (ESIpos): m/z=326 (M+H)⁺, LC-MS [Method 2]: Rₜ=1.49 min. ¹H-NMR (400 MHz, CD₃OD): δ 1.52 (s, 9H), 2.61 (s, 3H), 2.72 (t, 2H), 3.74 (t, 2H), 4.23 (d, 2H), 6.23 (br, 1H), 7.59 (d, 1H), 7.81 (s, 1H), 8.21 (d, 1H), 9.10 (s, 1H).

### Step 4: 7-Methyl-4-(1,2,3,6-tetrahydropyridin-4-yl)quinazoline

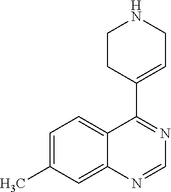 [SMILES: Cc1ccc2c(C3=CCNCC3)ncnc2c1]

This compound was synthesized by the same method as described in example 1 to give 55.7 mg of the product as a light green solid. MS (ESIpos): m/z=226 (M+H)⁺, LC-MS [Method 2]: Rₜ=0.69 min. ¹H-NMR (400 MHz, DMSO-d₆): δ 2.58 (s, 3H), 2.78 (t, 2H), 3.37 (t, 2H), 3.84-3.85 (m, 2H), 6.24 (br, 1H), 7.61 (d, 1H), 7.85 (s, 1H), 8.20 (d, 1H), 9.20 (s, 1H).

### Step 5: 7-Methyl-4-(piperidin-4-yl)quinazoline

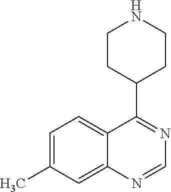 [SMILES: Cc1ccc2c(C3CCNCC3)ncnc2c1]

This compound was synthesized by the same method as described in example 1 to give 19.6 mg of the product as a light yellow solid. MS (ESIpos): m/z=228 (M+H)⁺, LC-MS [Method 2]: Rₜ=0.94 min. ¹H-NMR (400 MHz, DMSO-d₆): δ 1.78-1.79 (m, 4H), 2.56 (s, 3H), 2.85-3.07 (m, 3H), 3.78-4.13 (m, 2H), 7.59 (d, 1H), 7.79 (s, 1H), 8.30 (d, 1H), 9.16 (s, 1H).

### Step 6: [4-(7-Methylquinazolin-4-yl)piperidin-1-yl][4-(trifluoromethoxy)phenyl]-methanone

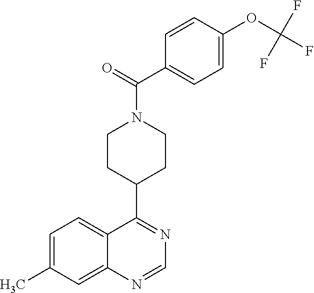 [SMILES: Cc1ccc2c(C3CCN(C(=O)c4ccc(OC(F)(F)F)cc4)CC3)ncnc2c1]

This compound was synthesized by the same method as described in example 9 to give 45.7 mg (35%) of the product as an off-white solid. MS (ESIpos): m/z=416 (M+H)⁺, LC-MS [Method 2]: Rₜ=2.68 min. ¹H-NMR (400 MHz, DMSO-d₆): δ 1.87-1.96 (m, 4H), 2.57 (s, 3H), 3.10-3.11 (m, 1H), 3.33-3.34 (m, 1H), 3.69-3.70 (m, 1H), 4.04-4.05 (m, 1H), 4.64-4.65 (m, 1H), 7.46 (d, 2H), 7.60-7.63 (m, 3H), 7.81 (s, 1H), 8.37 (d, 1H), 9.16 (s, 1H).

### Example 30

### [4-(6-Methylquinazolin-4-yl)piperidin-1-yl][4-(trifluoromethoxy)phenyl]-methanone

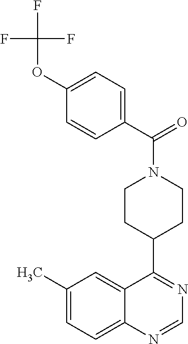 [SMILES: Cc1ccc2ncnc(C3CCN(C(=O)c4ccc(OC(F)(F)F)cc4)CC3)c2c1]

### Step 1: 6-Methylquinazolin-4(3H)-one

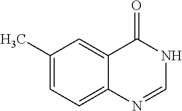

This compound was synthesized by the same method as described in example 1 to give 4.7 g (88%) of the product as a grey solid. MS: m/z=161 (M+H)⁺. LC-MS [Method 3]: Rₜ=1.09 min.

### Step 2: 4-Chloro-6-methyl-3,4-dihydroquinazoline

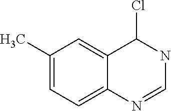

This compound was synthesized by the same method as described in example 1 to give 9.6 g (85%) of the product as a light yellow solid. MS (ESIpos): m/z=179 (M+H)⁺; LC-MS [Method 2]: Rₜ=0.96 min.

### Step 3: Tert-butyl 4-(6-methylquinazolin-4-yl)-5,6-dihydropyridine-1(2H)-carboxylate

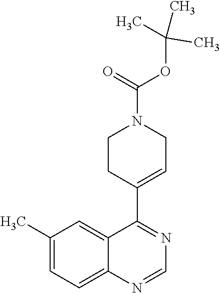 [SMILES: Cc1ccc2ncnc(C3=CCN(C(=O)OC(C)(C)C)CC3)c2c1]

This compound was synthesized by the same method as described in example 1 to give 32.6 mg of the product as a yellow solid. MS (ESIpos): m/z=326 (M+H)⁺; LC-MS [Method 3]: Rₜ=1.93 min; ¹H-NMR (300 MHz, DMSO-d₆): δ 1.47 (s, 9H), 2.55 (s, 3H), 2.66-2.67 (m, 2H), 3.63 (t, 2H), 4.15-4.16 (m, 2H), 6.27 (s, 1H), 7.84-7.95 (m, 2H), 8.08 (s, 1H), 9.16 (s, 1H).

### Step 4: 6-Methyl-4-(1,2,3,6-tetrahydropyridin-4-yl)quinazoline

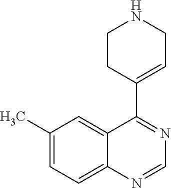 [SMILES: Cc1ccc2ncnc(C3=CCNCC3)c2c1]

This compound was synthesized by the same method as described in example 1 to give 540 mg (78%) of the product as a yellow solid. MS (ESIpos): m/z=226 (M+H)⁺; LC-MS [Method 2]: Rₜ=0.70 min; ¹H-NMR (400 MHz, DMSO-d₆): δ 2.56 (s, 3H), 2.74-2.78 (m, 2H), 3.30-3.33 (m, 2H), 3.79-3.80 (m, 2H), 6.26 (s, 1H), 7.89 (d, 1H), 7.96 (d, 1H), 8.09 (s, 1H), 9.20 (s, 1H).

### Step 5: 6-Methyl-4-(piperidin-4-yl)quinazoline

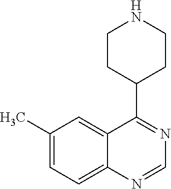 [SMILES: Cc1ccc2ncnc(C3CCNCC3)c2c1]

This compound was synthesized by the same method as described in in example 1 to give 10.8 mg of the product as a yellow solid. MS (ESIpos): m/z=228 (M+H)⁺; LC-MS [Method 2]: Rₜ=0.72 min; H-NMR (400 MHz, DMSO-d₆): δ 1.82-1.99 (m, 4H), 2.51 (s, 3H), 2.96-3.01 (m, 2H), 3.24-3.26 (m, 2H), 3.86-3.91 (m, 1H), 7.79 (d, 1H), 7.85 (d, 1H), 8.16 (s, 1H), 9.09 (s, 1H).

### Step 6: (4-(6-Methylquinazolin-4-yl)piperidin-1-yl)(4-(trifluoromethoxy)phenyl)-methanone

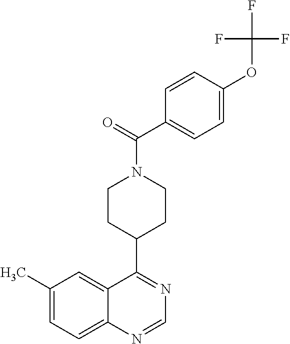 [SMILES: Cc1ccc2ncnc(C3CCN(C(=O)c4ccc(OC(F)(F)F)cc4)CC3)c2c1]

This compound was synthesized by the same method as described in in example 9 to give 41.5 mg (28%) of the product as a white solid. MS (ESIpos): m/z=416 (M+H)⁺; LC-MS [Method 2]: Rₜ=1.53 min; ¹H-NMR (300 MHz, DMSO-d₆): δ 1.87-1.98 (m, 4H), 2.58 (s, 3H), 3.11-3.16 (m, 1H), 3.38-3.39 (m, 1H), 3.70-3.71 (m, 1H), 4.02-4.09 (m, 1H), 4.65-4.66 (m, 1H), 7.46 (d, 2H), 7.61 (d, 2H), 7.85 (d, 1H), 7.92 (d, 1H), 8.26 (s, 1H), 9.16 (s, 1H).

### Example 31

### Methyl 4-{1-[4-(trifluoromethoxy)benzoyl]piperidin-4-yl}quinazoline-7-carboxylate

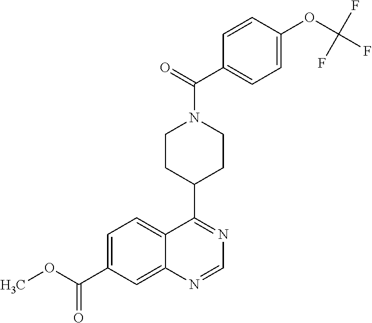 [SMILES: COC(=O)c1ccc2c(C3CCN(C(=O)c4ccc(OC(F)(F)F)cc4)CC3)ncnc2c1]

### Step 1: 2-Aminoterephthalic Acid

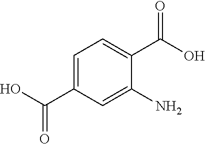 [SMILES: Nc1cc(C(=O)O)ccc1C(=O)O]

Dimethyl 2-aminoterephthalate (7.0 g, 33.5 mmol) was dissolved in 60 mL of methanol/water (5:1, v:v). Then sodium hydroxide, 5.35 g (133.8 mmol), was added to the above solution and the resulting mixture was stirred at 60° C. for 3 hours. The solvent was removed in vacuo and the residue was re-dissolved with water. Hydrochloric acid (6 mol/L) was added to adjust the pH value to 3 and the resulting mixture was extracted with ethyl acetate. The combined organic layer was dried over sodium sulfate and the solvent was removed in vacuo to give 6.00 g (94%) of the product as a yellow solid. MS (ESIpos): m/z=182 (M+H)⁺, LC-MS [Method 3]: Rₜ=0.25 min.

### Step 2: 4-Oxo-3,4-dihydroquinazoline-7-carboxylic Acid

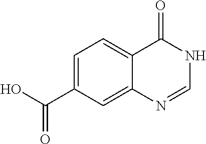 [SMILES: O=C(O)c1ccc2c(=O)[nH]cnc2c1]

This compound was synthesized by the same method as described in in example 1 to give 3.30 g (94%) of the product as a yellow solid. MS (ESIpos): m/z=191 (M+H)⁺. LC-MS [Method 2]: Rₜ=0.56 min.

### Step 3: Methyl 4-oxo-3,4-dihydroquinazoline-7-carboxylate

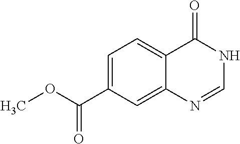

To a solution of 4-oxo-3,4-dihydroquinazoline-7-carboxylic acid (3.30 g, 14.8 mmol) in 60 mL methanol was added 6.6 mL of sulfuric acid. The resulting mixture was stirred at 60° C. for 11 hours. After cooled to room temperature, the solvent was removed in vacuo and the residue was diluted with water. Aqueous sodium hydroxide (6 mol/L) was added to adjust the pH value to 8 and the precipitated solid was collected by filtration. The filter cake was washed with water and dried in oven to give 3.00 g (86%) of the product as a light yellow solid. MS (ESIpos): m/z=205 (M+H)⁺. LC-MS [Method 2]: Rₜ=0.67 min.

### Step 4: Methyl 4-chloroquinazoline-7-carboxylate

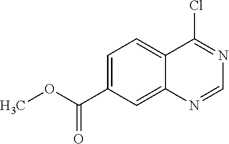

This compound was synthesized by the same method as described in in example 1 to give 2.80 g (81%) of the product as a light brown solid. MS (ESIpos): m/z=223 (M+H)⁺. LC-MS [Method 1]: Rₜ=0.89 min.

### Step 5: 4-[1-(Tert-butoxycarbonyl)-1,2,3,6-tetrahydropyridin-4-yl]quinazoline-7-carboxylic Acid

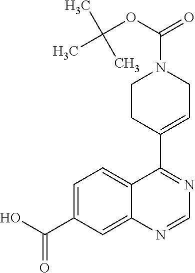 [SMILES: CC(C)(C)OC(=O)N1CC=C(c2ncnc3cc(C(=O)O)ccc23)CC1]

This compound was synthesized by the same method as described in in example 1 to give 4.00 g (71%) of the product as a yellow solid. MS (ESIpos): m/z=356 (M+H)⁺. LC-MS [Method 2]: Rₜ=2.25 min. ¹H-NMR (400 MHz, DMSO-d₆): δ 1.46 (s, 9H), 2.70 (t, 2H), 3.65 (t, 2H), 4.17 (d, 2H), 6.34 (s, 1H), 8.15 (d, 1H), 8.44 (d, 1H), 8.51 (s, 1H), 9.33 (s, 1H), 13.70 (br, 1H).

### Step 6: Methyl 4-(1,2,3,6-tetrahydropyridin-4-yl)quinazoline-7-carboxylate

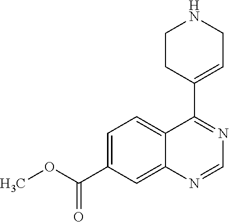 [SMILES: COC(=O)c1ccc2c(C3=CCNCC3)ncnc2c1]

To a solution of 4-[1-(Tert-butoxycarbonyl)-1,2,3,6-tetrahydropyridin-4-yl]quinazoline-7-carboxylic acid (4.0 g, 7.2 mmol) in 60 mL methanol was added 8.0 mL of sulfuric acid. The resulting mixture was stirred at 60° C. for 13 hours. After cooled to room temperature, the solvent was removed in vacuo and the residue was diluted with water. Aqueous sodium hydroxide (6 mol/L) was added to adjust the pH value to 10. The resulting mixture was extracted with dichloromethane and the combined organic layer was dried over sodium sulfate. The solvent was removed in vacuo to give 2.00 g (92%) of the product as a brown solid. MS (ESIpos): m/z=270 (M+H)⁺, LC-MS [Method 2]: Rₜ=0.75 min. ¹H-NMR (400 MHz, DMSO-d₆): δ 2.67 (t, 2H), 3.14-3.16 (m, 2H), 3.65-3.66 (m, 2H), 3.97 (s, 3H), 6.33 (s, 1H), 8.17 (d, 1H), 8.30 (s, 1H), 8.45 (d, 1H), 8.53 (s, 1H), 9.35 (s, 1H).

### Step 7: Methyl 4-{1-[4-(trifluoromethoxy)benzoyl]-1,2,3,6-tetrahydropyridin-4-yl}quinazoline-7-carboxylate

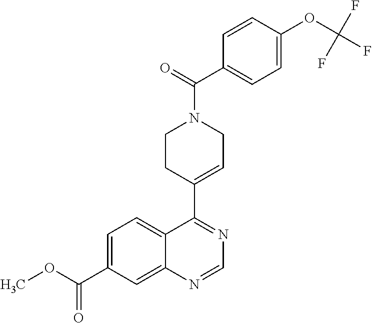 [SMILES: COC(=O)c1ccc2c(C3=CCN(C(=O)c4ccc(OC(F)(F)F)cc4)CC3)ncnc2c1]

This compound was synthesized by the same method as described in in example 9 to give 1.7 g (65%) of product as a light yellow solid. MS (ESIpos): m/z=458 (M+H)⁺, LC-MS [Method 2]: Rₜ=1.57 min. ¹H-NMR (400 MHz, DMSO-d₆): δ 2.80 (t, 2H), 3.63-3.64 (m, 1H), 3.96-3.98 (m, 4H), 4.26-4.45 (m, 2H), 6.26 (s, 0.5H), 6.45 (s, 0.5H), 7.48-7.50 (m, 2H), 7.66-7.68 (m, 2H), 8.16 (s, 1H), 8.53-8.54 (m, 2H), 9.38 (s, 1H).

### Step 8: Methyl 4-{1-[4-(trifluoromethoxy)benzoyl]piperidin-4-yl}quinazoline-7-carboxylate

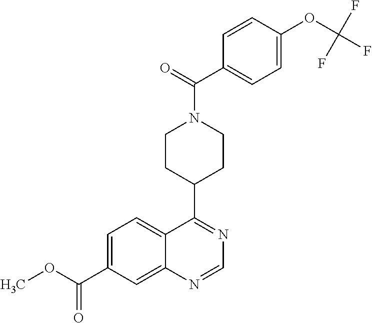 [SMILES: COC(=O)c1ccc2c(C3CCN(C(=O)c4ccc(OC(F)(F)F)cc4)CC3)ncnc2c1]

To a solution of methyl 4-{1-[4-(trifluoromethoxy)benzoyl]-1,2,3,6-tetrahydropyridin-4-yl}quinazoline-7-carboxylate (1.70 g, 3.2 mmol) in 70 mL methanol was added palladium/carbon (0.34 g, 0.3 mmol). The resulting mixture was stirred at room temperature for 5 hours under hydrogen atmosphere (3 atm). The solid was removed by filtration and the filtrate was concentrated in vacuo. The residue was purified by chromatography to give 1.00 g (61%) of the product as a yellow solid. MS (ESIpos): m/z=460 (M+H)⁺. LC-MS [Method 3]: Rₜ=1.95 min. ¹H-NMR (400 MHz, DMSO-d₆): δ 1.90-2.01 (m, 4H), 3.11-3.12 (m, 1H), 3.33-3.34 (m, 1H), 3.71-3.72 (m, 1H), 3.97 (s, 3H), 4.13-4.15 (m, 1H), 4.65-4.66 (m, 1H), 7.45-7.47 (m, 2H), 7.59-7.60 (m, 2H), 8.20 (d, 1H), 8.53 (s, 1H), 8.64 (d, 1H), 9.35 (s, 1H).

### Example 32

### 4-{1-[4-(Trifluoromethoxy)benzoyl]piperidin-4-yl}quinazoline-7-carboxylic Acid

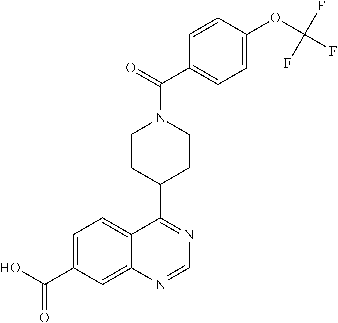 [SMILES: O=C(O)c1ccc2c(C3CCN(C(=O)c4ccc(OC(F)(F)F)cc4)CC3)ncnc2c1]

Methyl 4-{1-[4-(trifluoromethoxy)benzoyl]piperidin-4-yl}quinazoline-7-carboxylate (1 g, 1.6 mmol) was dissolved in 21 mL of methanol/water (v:v=5:2), then sodium hydroxide 0.19 g (4.8 mmol), was added at 0° C., then the resulting mixture was stirred at room temperature for 2 h. After removal of the solvent, the resulting mixture was diluted by addition of water and washed with ethyl acetate, the water phase was obtained. The pH value of water phase was adjusted to 3 with hydrogen chloride (3 mol/L) and the mixture was extracted with ethyl acetate. The combined organic layer was washed with water, dried over sodium sulfate and evaporated to dryness to give 0.9 g (90%) of the product as a light yellow solid.

MS (ESIpos): m/z=446 (M+H)⁺. LC-MS [Method 3]: Rₜ=1.25 min. ¹H-NMR (400 MHz, DMSO-d₆): δ 1.89-2.01 (m, 4H), 3.11-3.15 (m, 1H), 3.33-3.41 (m, 1H), 3.70-3.76 (m, 1H), 4.11-4.17 (m, 1H), 4.64-4.66 (m, 1H), 7.45-7.50 (m, 2H), 7.59-7.61 (m, 2H), 8.06 (d, 1H), 8.51 (s, 1H), 8.59-8.61 (d, 1H), 9.32 (s, 2H), 13.64 (br, 1H).

### Example 33

### 4-{1-[4-(Trifluoromethoxy)benzoyl]piperidin-4-yl}quinazoline-7-carboxamide

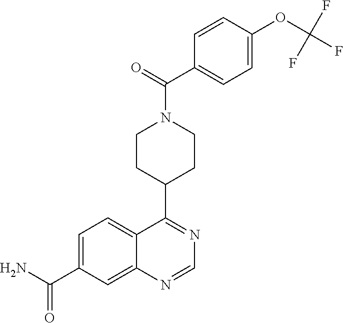 [SMILES: NC(=O)c1ccc2c(C3CCN(C(=O)c4ccc(OC(F)(F)F)cc4)CC3)ncnc2c1]

Methyl 4-{1-[4-(trifluoromethoxy)benzoyl]piperidin-4-yl}quinazoline-7-carboxylate (400 mg, 0.8 mmol) was added to 30 mL of ammonia/methanol (7 M). The resulting mixture was stirred at room temperature for 26 hours. The solvent was removed in vacuo and the residue was purified by chromatography to give 300 mg (70%) of the product as a light yellow solid. MS (ESIpos): m/z=445 (M+H)⁺. LC-MS [Method 3]: Rₜ=1.57 min. ¹H-NMR (400 MHz, DMSO-d₆): δ 1.90-2.00 (m, 4H), 3.12-3.13 (m, 1H), 3.33-3.38 (m, 1H), 3.69-3.70 (m, 1H), 4.11-4.12 (m, 1H), 4.64-4.65 (m, 1H), 7.45-7.47 (m, 2H), 7.59-7.61 (m, 2H), 7.76 (s, 1H), 8.17 (d, 1H), 8.41 (s, 1H), 8.55-8.56 (m, 2H), 9.30 (s, 1H).

### Example 34

### 4-{1-[4-(Trifluoromethoxy)benzoyl]piperidin-4-yl}quinazoline-7-carbonitrile

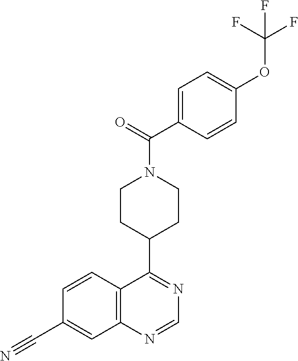

To a solution of 4-{1-[4-(trifluoromethoxy)benzoyl]piperidin-4-yl}quinazoline-7-carboxamide (150 mg, 0.3 mmol) in 10 mL tetrahydrofuran were added pyridine (0.25 mL, 3.0 mmol) and trifluoroacetic anhydride (0.4 mL, 3.0 mmol). The resulting mixture was stirred at room temperature for 4 hours. The solvent was removed in vacuo and the residue was re-dissolved with ethyl acetate. The resulting solution was washed with water and the solvent was removed in vacuo. The residue was purified by chromatography to give 48.5 mg (36%) of the product as an off-white solid. MS (ESIpos): m/z=427 (M+H)⁺. LC-MS [Method 3]: Rₜ=1.92 min. ¹H-NMR (400 MHz, DMSO-d₆): δ 1.88-1.99 (m, 4H), 3.10-3.11 (m, 1H), 3.33-3.34 (m, 1H), 3.68-3.69 (m, 1H), 4.13-4.14 (m, 1H), 4.63-4.64 (m, 1H), 7.46 (d, 2H), 7.60 (d, 2H), 8.15 (d, 1H), 8.65 (s, 1H), 8.70 (d, 1H), 9.38 (s, 1H).

### Example 35

### [4-(7-Methoxyquinazolin-4-yl)piperidin-1-yl][4-(trifluoromethoxy)phenyl]-methanone

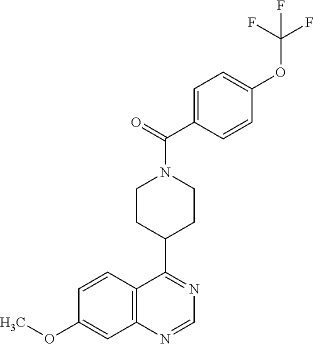

### Step 1: 7-Methoxyquinazolin-4(3H)-one

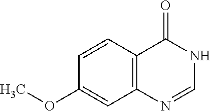

This compound was synthesized by the same method as described in example 1 to give 3.16 g (95%) of the product as a brown solid. MS (ESIpos): m/z=177 (M+H)⁺; LC-MS [Method 1]: Rₜ=0.58 min.

### Step 2: 4-Chloro-7-methoxyquinazoline

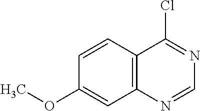

This compound was synthesized by the same method as described in example 1 to give 2.38 g (62%) of the product as a brown solid. MS (ESIpos): m/z=195 (M+H)⁺; LC-MS [Method 2]: Rₜ=0.91 min.

### Step 3: Tert-butyl 4-(7-methoxyquinazolin-4-yl)-5,6-dihydropyridine-1(2H)-carboxylate

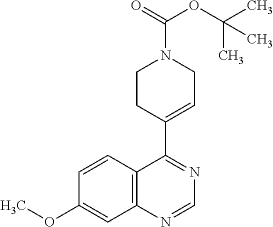 [SMILES: COc1ccc2c(C3=CCN(C(=O)OC(C)(C)C)CC3)ncnc2c1]

This compound was synthesized by the same method as described in example 1 to give 2.35 g (88%) of the product as a light yellow solid. MS (ESIpos): m/z=342 (M+H)⁺; LC-MS [Method 2]: Rₜ=1.38 min. ¹H-NMR (400 MHz, DMSO-d₆): δ 1.46 (s, 9H), 2.63-2.64 (m, 2H), 3.62 (t, 2H), 3.97 (s, 3H), 4.14-3-4.15 (m, 2H), 6.21 (br, 1H), 7.30-7.33 (m, 1H), 7.38 (s, 1H), 8.20 (d, 1H), 9.11 (s, 1H).

### Step 4: 7-Methoxy-4-(1,2,3,6-tetrahydropyridin-4-yl)quinazoline

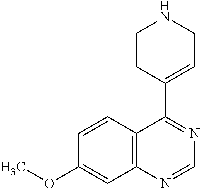 [SMILES: COc1ccc2c(C3=CCNCC3)ncnc2c1]

This compound was synthesized by the same method as described in example 1 to give 0.51 g (69%) of the product as an off-white solid. MS (ESIpos): m/z=242 (M+H)⁺; LC-MS [Method 2]: Rₜ=0.65 min. ¹H NMR (400 MHz, DMSO-d₆): δ 2.51-2.57 (m, 2H), 2.95-3.05 (m, 1H), 3.45-3.53 (m, 3H), 3.97 (s, 3H), 4.09-4.15 (m, 1H), 6.20 (br, 1H), 7.30-7.33 (m, 1H), 7.37 (s, 1H), 8.19 (d, 1H), 9.10 (s, 1H).

### Step 5: 7-Methoxy-4-(piperidin-4-yl)quinazoline

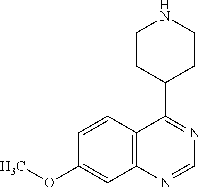 [SMILES: COc1ccc2c(C3CCNCC3)ncnc2c1]

This compound was synthesized by the same method as described in example 1 to give 0.18 g (88%) of the product as an off-white solid. MS (ESIpos): m/z=244 (M+H)⁺; LC-MS [Method 2]: Rₜ=0.66 min. ¹H-NMR (400 MHz, DMSO-d₆): δ 1.72-1.78 (m, 4H), 2.79-3.07 (m, 3H), 3.78-3.79 (m, 1H), 3.96 (s, 3H), 4.12-4.14 (m, 1H), 7.33-7.35 (m, 2H), 8.33 (d, 1H), 9.09 (s, 1H).

### Step 6: (4-(7-Methoxyquinazolin-4-yl)piperidin-1-yl)(4-(trifluoromethoxy)phenyl)-methanone

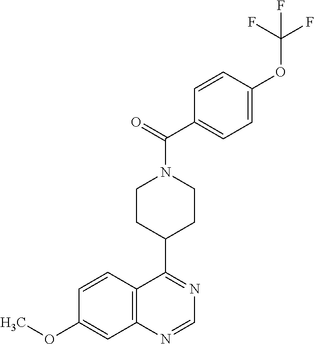 [SMILES: COc1ccc2c(C3CCN(C(=O)c4ccc(OC(F)(F)F)cc4)CC3)ncnc2c1]

This compound was synthesized by the same method as described in example 9 to give 42.3 mg (24%) of the product as an off-white solid. MS (ESIpos): m/z=432 (M+H)⁺; LC-MS [Method 2]: Rₜ=1.42 min. ¹H-NMR (400 MHz, DMSO-d₆): δ 1.83-1.92 (m, 4H), 3.05-3.13 (m, 1H), 3.33-3.37 (m, 1H), 3.65-3.72 (m, 1H), 3.97-4.03 (m, 4H), 4.62-4.66 (m, 1H), 7.35-7.38 (m, 2H), 7.45-7.47 (m, 2H), 7.57-7.60 (m, 2H), 8.38 (d, 1H), 9.11 (s, 1H).

### Example 36

### [4-(7-Hydroxyquinazolin-4-yl)piperidin-1-yl][4-(trifluoromethoxy)phenyl]-methanone

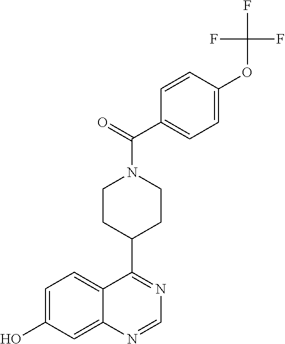 [SMILES: O=C(c1ccc(OC(F)(F)F)cc1)N1CCC(c2ncnc3cc(O)ccc23)CC1]

### Step 1: 4-(Piperidin-4-yl)quinazolin-7-ol

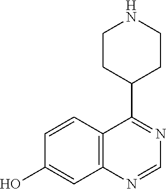 [SMILES: Oc1ccc2c(C3CCNCC3)ncnc2c1]

To a solution of 7-methoxy-4-(piperidin-4-yl)quinazoline (1.2 g, 4.93 mmol) in 10 mL of N,N-dimethylformamide was added sodium ethanethiolate (2.07 g, 24.66 mmol). The resulting mixture was stirred at 130° C. for 2 hours. After cooled to room temperature, the solvent was removed in vacuo and the residue was washed with methanol/dichloromethane for several times. The combined organic layer was concentrated in vacuo to give 1.0 g (crude) of the product as a semi-solid. MS (ESIpos): m/z=230 (M+H)⁺; LC-MS [Method 1]: Rₜ=4.03 min. ¹H-NMR (300 MHz, DMSO-d₆): δ1.82-1.97 (m, 4H), 2.95-2.99 (m, 2H), 3.23-3.27 (m, 2H), 3.80 (m, 1H), 7.15 (s, 1H), 7.24-7.28 (m, 1H), 8.24-8.27 (m, 1H), 8.39 (br, 1H), 9.00 (s, 1H), 9.84 (br, 1H).

### Step 2: (4-(7-Hydroxyquinazolin-4-yl)piperidin-1-yl)(4-(trifluoromethoxy)phenyl)-methanone

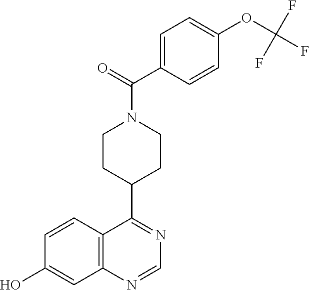 [SMILES: O=C(c1ccc(OC(F)(F)F)cc1)N1CCC(c2ncnc3cc(O)ccc23)CC1]

This compound was synthesized by the same method as described in in example 9 to give 700 mg of the product as a white solid. MS (ESIpos): m/z=418 (M+H)⁺. LC-MS [Methode 2]: Rₜ=0.93 min. ¹H-NMR (400 MHz, DMSO-d₆): δ 1.84-1.86 (m, 4H), 3.05 (m, 2H), 3.66 (m, 1H), 3.89-3.96 (m, 1H), 4.61 (m, 1H), 7.16 (s, 1H), 7.25 (d, 1H), 7.43 (d, 2H), 7.56 (d, 2H), 8.30 (d, 1H), 8.99 (s, 1H), 9.83 (br, 1H).

### Example 37

### [4-(7-Aminoquinazolin-4-yl)piperidin-1-yl][4-(trifluoromethoxy)phenyl]-methanone

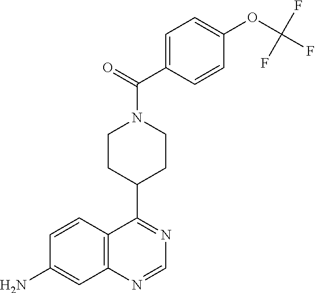 [SMILES: Nc1ccc2c(C3CCN(C(=O)c4ccc(OC(F)(F)F)cc4)CC3)ncnc2c1]

### Step 1: Tert-butyl (4-{1-[4-(trifluoromethoxy)benzoyl]piperidin-4-yl}quinazolin-7-yl)carbamate

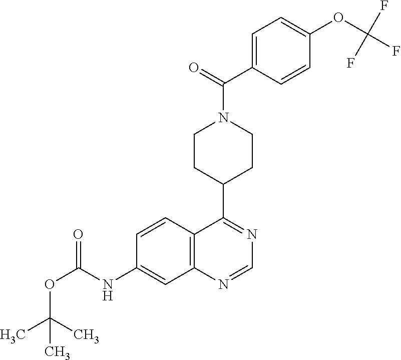 [SMILES: CC(C)(C)OC(=O)Nc1ccc2c(C3CCN(C(=O)c4ccc(OC(F)(F)F)cc4)CC3)ncnc2c1]

To a solution of 4-{1-[4-(trifluoromethoxy)benzoyl]piperidin-4-yl}quinazoline-7-carboxylic acid (200 mg, 0.4 mmol) 5 mL of tert-butanol were added diphenyl phosphoroazidate (115 mg, 0.4 mmol) and triethylamine (42 mg, 0.4 mmol). The resulting mixture was stirred at 80° C. for 15 h. The solvent was removed in vacuo, the residue was purified by chromatography to give 150 mg (65%) of the product as light yellow solid. MS (ESIpos): m/z=517 (M+H)⁺.

### Step 2: [4-(7-Aminoquinazolin-4-yl)piperidin-1-yl][4-(trifluoromethoxy)phenyl]-methanone

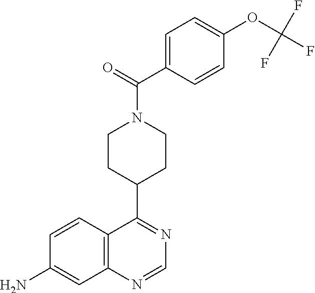 [SMILES: Nc1ccc2c(C3CCN(C(=O)c4ccc(OC(F)(F)F)cc4)CC3)ncnc2c1]

This compound was synthesized by the same method as described in in example 1 to give 13.2 mg (22%) of the product as an off-white solid. MS (ESIpos): m/z=417 (M+H)⁺. LC-MS [Methode 3]: Rₜ=1.64 min. ¹H-NMR (400 MHz, DMSO-d₆): δ 1.75-1.86 (m, 4H), 2.99-3.03 (m, 1H), 3.31-3.32 (m, 1H), 3.64-3.71 (m, 1H), 3.78-3.82 (m, 1H), 4.61-4.68 (m, 1H), 6.34 (s, 2H), 6.79 (s, 1H), 7.05 (d, 1H), 7.45 (d, 2H), 7.58 (d, 2H), 8.08 (d, 1H), 8.80 (s, 1H).

### Example 38

### N-(4-{1-[4-(Trifluoromethoxy)benzoyl]piperidin-4-yl}quinazolin-7-yl)acetamide

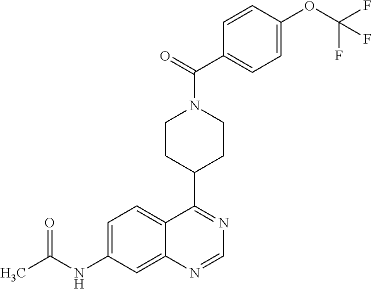 [SMILES: CC(=O)Nc1ccc2c(C3CCN(C(=O)c4ccc(OC(F)(F)F)cc4)CC3)ncnc2c1]

[4-(7-Aminoquinazolin-4-yl)piperidin-1-yl][4-(trifluoromethoxy)phenyl]methanone (30 mg, 0.72 mmol) and triethylamine (22 mg, 0.22 mmol), were dissolved in 0.5 mL of dichloromethane. Acetyl chloride (8 mg, 0.11 mmol) was added dropwise to the solution and the resulting mixture was stirred at room temperature for 10 min. The solvent was removed in vacuo and the residue was purified by chromatography to give 5.3 mg (16%) of the product as a white solid. MS (ESIpos): m/z=459 (M+H)⁺. LC-MS [Methode 2]: Rₜ=1.27 min. ¹H-NMR (400 MHz, DMSO-d₆): δ 1.62-1.91 (m, 4H), 2.13 (s, 3H), 3.00-3.15 (m, 1H), 3.28-3.30 (m, 1H), 3.65-3.74 (m, 1H), 3.92-4.00 (m, 1H), 4.60-4.65 (m, 1H), 7.44 (d, 2H), 7.57 (d, 2H), 7.78 (d, 1H), 8.37-8.40 (m, 2H), 9.09 (s, 1H), 10.51 (s, 1H).

### Example 39

### N-(4-{1-[4-(Trifluoromethoxy)benzoyl]piperidin-4-yl}quinazolin-7-yl)methane-sulfonamide

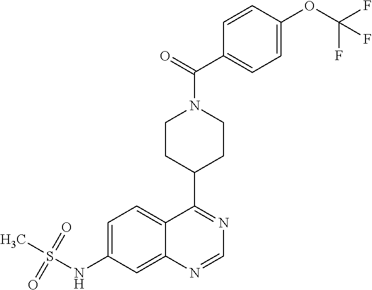 [SMILES: CS(=O)(=O)Nc1ccc2c(C3CCN(C(=O)c4ccc(OC(F)(F)F)cc4)CC3)ncnc2c1]

[4-(7-Aminoquinazolin-4-yl)piperidin-1-yl][4-(trifluoromethoxy)phenyl]methanone (30 mg, 0.72 mmol) and triethylamine, 36 mg (0.36 mmol) were added to 0.5 mL dichloromethane. After dropwise addition of methanesulfonyl chloride (21 mg, 0.18 mmol), the resulting mixture was stirred at room temperature for 10 min. The solvent was removed in vacuo and the residue was dissolved with methanol. Then aqueous sodium hydroxide (0.2 mL, 4M), was added and the resulting mixture was stirred at room temperature for another 30 min. The solvent was removed in vacuo and the residue was purified by chromatography to give 8.6 mg (22%) of the product as a yellow solid. MS (ESIpos): m/z=495 (M+H)⁺. LC-MS [Methode 2]: Rₜ=1.27 min. ¹H-NMR (300 MHz, CD₃OD): δ 1.87-2.04 (m, 4H), 3.11 (s, 3H), 3.14-3.20 (m, 1H), 3.40-3.49 (m, 1H), 3.80-3.95 (m, 1H), 3.96-4.09 (m, 1H), 4.70-4.77 (m, 1H), 7.37 (d, 2H), 7.54-7.59 (m, 3H), 7.76 (s, 1H), 8.37 (d, 1H), 9.04 (s, 1H).

### Example 40

### [4-(7-Bromoquinazoline-4-yl)piperidin-1-yl][4-(trifluoromethoxy)phenyl]-methanone

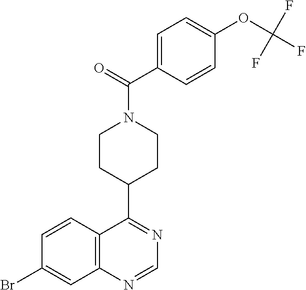 [SMILES: O=C(c1ccc(OC(F)(F)F)cc1)N1CCC(c2ncnc3cc(Br)ccc23)CC1]

To a solution of [4-(7-aminoquinazolin-4-yl)piperidin-1-yl][4-(trifluoromethoxy)phenyl]-methanone (100 mg, 0.24 mmol) in 5 mL acetonitrile were added copper bromide (41 mg, 0.29 mmol) and isoamyl nitrite (45 mg, 0.38 mmol). The resulting mixture was stirred at room temperature for 24 hours under nitrogen atmosphere. The solvent was removed in vacuo and the residue was purified by chromatography to give 10 mg (9%) of the product as an off-white solid. MS (ESIpos): m/z=480 (M+H)⁺. LC-MS [Method 3]: Rₜ=2.07 min. ¹H-NMR (400 MHz, CDCl₃): δ 1.94-2.11 (m, 4H), 3.10-3.24 (m, 2H), 3.70-3.85 (m, 1H), 3.90-4.19 (m, 1H), 4.70-5.10 (m, 1H), 7.26-7.29 (m, 2H), 7.51-7.54 (m, 2H), 7.74 (d, 1H), 8.16-8.34 (m, 2H), 9.44 (s, 1H).

### Example 41

### N-Methyl-4-{1-[4-(trifluoromethoxy)benzoyl]piperidin-4-yl}quinazoline-7-carboxamide

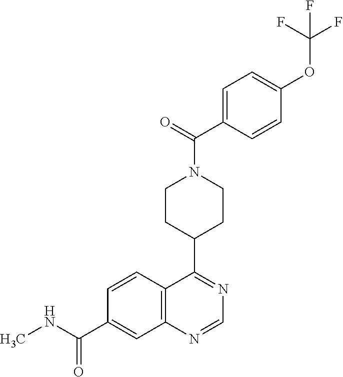 [SMILES: CNC(=O)c1ccc2c(C3CCN(C(=O)c4ccc(OC(F)(F)F)cc4)CC3)ncnc2c1]

This compound was synthesized by the same method as described in in example 1 to give 45.9 mg (55%) of the product as a yellow solid. MS (ESIpos): m/z=459 (M+H)⁺; LC-MS [Method 3]: Rₜ=2.59 min. ¹H-NMR (400 MHz, DMSO-d₆): δ 1.87-2.08 (m, 4H), 2.87 (d, 3H), 3.04-3.11 (m, 1H), 3.33-3.39 (m, 1H), 3.70-3.72 (m, 1H), 4.11-4.15 (m, 1H), 4.65-4.72 (m, 1H), 7.46 (d, 2H), 7.59 (d, 2H), 8.14 (d, 1H), 8.47 (s, 1H), 8.57 (d, 1H), 8.89-8.90 (m, 1H), 9.36 (s, 1H).

### Example 42

### N,N-Dimethyl-4-{1-[4-(trifluoromethoxy)benzoyl]piperidin-4-yl}quinazoline-7-carboxamide

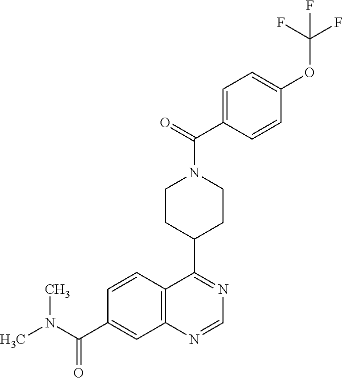 [SMILES: CN(C)C(=O)c1ccc2c(C3CCN(C(=O)c4ccc(OC(F)(F)F)cc4)CC3)ncnc2c1]

This compound was synthesized by the same method as described in example 1 (to give 36.9 mg (43%) of the product as a light yellow solid. MS (ESIpos): m/z=473 (M+H)⁺; LC-MS [Method 3]: Rₜ=3.37 min. ¹H-NMR (400 MHz, DMSO-d₆): δ 1.88-2.00 (m, 4H), 2.93 (s, 3H), 3.06 (s, 3H), 3.10-3.22 (m, 1H), 3.30-3.38 (m, 1H), 3.70-3.71 (m, 1H), 4.06-4.14 (m, 1H), 4.64-4.70 (m, 1H), 7.46 (d, 2H), 7.60 (d, 2H), 7.75 (d, 1H), 7.99 (s, 1H), 8.56 (d, 1H), 9.27 (s, 1H).

### Example 43

### Pyrrolidin-1-yl(4-{1-[4-(trifluoromethoxy)benzoyl]piperidin-4-yl}quinazolin-7-yl)methanone

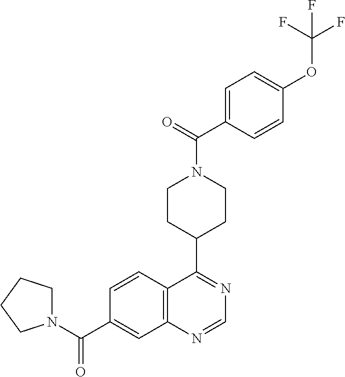 [SMILES: O=C(c1ccc2c(C3CCN(C(=O)c4ccc(OC(F)(F)F)cc4)CC3)ncnc2c1)N1CCCC1]

This compound was synthesized by the same method as described in example 1 to give 26.1 mg (29%) of the product as a light yellow solid. MS (ESIpos): m/z=499 (M+H)⁺; LC-MS [Method 3]: Rₜ=1.76 min. ¹H-NMR (400 MHz, DMSO-d₆): δ 1.82-1.94 (m, 7H), 3.06-3.12 (m, 1H), 3.40-3.43 (m, 4H), 3.52-3.55 (m, 2H), 3.65-3.70 (m, 1H), 4.04-4.15 (m, 1H), 4.64-4.72 (m, 1H), 7.42 (d, 2H), 7.60 (d, 2H), 7.83 (d, 1H), 8.09 (s, 1H), 8.55 (d, 1H), 9.28 (s, 1H).

### Example 44

### Morpholin-4-yl(4-{1-[4-(trifluoromethoxy)benzoyl]piperidin-4-yl}quinazolin-7-yl)methanone

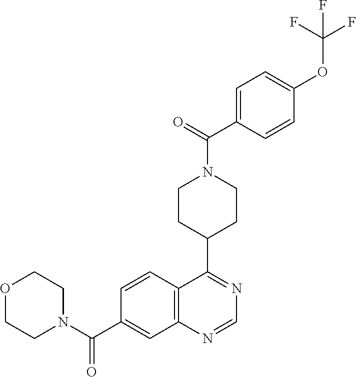 [SMILES: O=C(c1ccc2c(C3CCN(C(=O)c4ccc(OC(F)(F)F)cc4)CC3)ncnc2c1)N1CCOCC1]

This compound was synthesized by the same method as described in example 1 to give 47.0 mg (51%) of the product as a light yellow solid. MS (ESIpos): m/z=515 (M+H)⁺; LC-MS [Method 3]: Rₜ=1.66 min. ¹H-NMR (400 MHz, DMSO-d₆): δ 1.88-2.08 (m, 4H), 3.06-3.12 (m, 1H), 3.33-3.35 (m, 3H), 3.52-3.57 (m, 2H), 3.68-3.72 (m, 5H), 4.04-4.15 (m, 1H), 4.64-4.72 (m, 1H), 7.46 (d, 2H), 7.60 (d, 2H), 7.77 (d, 1H), 8.01 (s, 1H), 8.57 (d, 1H), 9.28 (s, 1H).

### Example 45

### N-Phenyl-4-{1-[4-(trifluoromethoxy)benzoyl]piperidin-4-yl}quinazoline-7-carboxamide

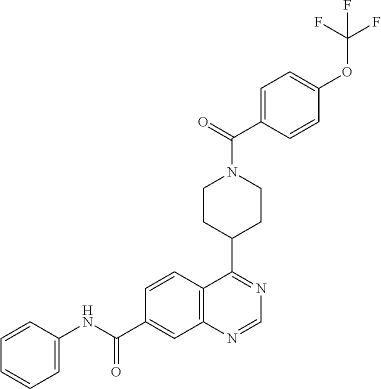 [SMILES: O=C(Nc1ccccc1)c1ccc2c(C3CCN(C(=O)c4ccc(OC(F)(F)F)cc4)CC3)ncnc2c1]

This compound was synthesized by the same method as described in example 1 to give 48.3 mg (51%) of the product as a light yellow solid. MS (ESIpos): m/z=521 (M+H)⁺; LC-MS [Method 3]: Rₜ=1.97 min. ¹H-NMR (400 MHz, DMSO-d₆): δ 1.90-2.04 (m, 4H), 3.10-3.16 (m, 1H), 3.33-3.42 (m, 1H), 3.66-3.74 (m, 1H), 4.13-4.18 (m, 1H), 4.62-4.68 (m, 1H), 7.16 (t, 1H), 7.39-7.48 (m, 4H), 7.61 (d, 2H), 7.85 (d, 2H), 8.23 (m, 1H), 8.64 (d, 2H), 9.36 (s, 1H), 10.68 (s, 1H).

### Example 46

### {4-[7-(2-Methylpropoxy)quinazolin-4-yl]piperidin-1-yl}[4-(trifluoromethoxy)-phenyl]methanone

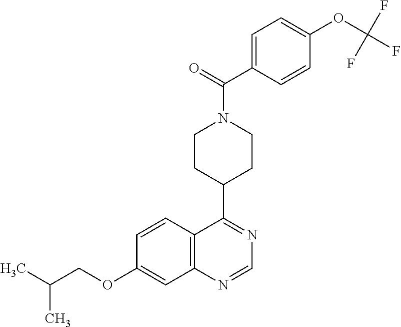 [SMILES: CC(C)COc1ccc2c(C3CCN(C(=O)c4ccc(OC(F)(F)F)cc4)CC3)ncnc2c1]

To a solution of [4-(7-Hydroxyquinazolin-4-yl)piperidin-1-yl][4-(trifluoromethoxy)phenyl]methanone (90 mg, 0.22 mmol) in 1 mL of N,N-dimethyl formamide were added potassium carbonate, (60 mg, 0.43 mmol), in portions at 0° C. for 30 min 1-iodo-2-methylpropane (119 mg, 0.65 mmol). The resulting solution was stirred 13 h at room temperature. After chromatography, the title compound was obtained as a white solid (18 mg, 17%).

MS (ESIpos): m/z=474 (M+H)⁺; LC-MS [Method 2]: Rₜ=1.76 min; ¹H-NMR (400 MHz, DMSO-d₆): δ 1.01 (d, 6H), 1.84-1.89 (m, 4H), 2.04-2.12 (m, 1H), 3.06-3.07 (m, 1H), 3.38-3.39 (m, 1H), 3.68-3.69 (m, 1H), 3.95-3.96 (m, 3H), 4.61-4.62 (m, 1H), 7.32-7.37 (m, 2H), 7.43-7.45 (m, 2H), 7.56-7.58 (m, 2H), 8.36 (d, 1H), 9.08 (s, 1H).

### Example 47

### {4-[7-(2-Hydroxyethoxy)quinazolin-4-yl]piperidin-1-yl}[4-(trifluoromethoxy)-phenyl]methanone

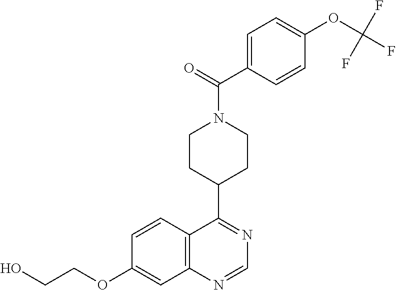 [SMILES: O=C(c1ccc(OC(F)(F)F)cc1)N1CCC(c2ncnc3cc(OCCO)ccc23)CC1]

This compound was synthesized by the same method as described in example 46. The reaction mixture was stirred at 50° for 24 h to give 22.4 mg (20%) of the product as a white solid after chromatography. MS (ESIpos): m/z=462 (M+H)⁺; LC-MS [Method 2]: Rₜ=1.08 min; ¹H-NMR (400 MHz, DMSO-d₆): δ 1.86-1.89 (m, 4H), 3.08-3.09 (m, 1H), 3.37-3.80 (m, 1H), 3.69-3.70 (m, 1H), 3.78-3.82 (m, 2H), 3.98-3.99 (m, 1H), 4.20-4.22 (m, 2H), 4.62-4.63 (m, 1H), 4.95-4.97 (m, 1H), 7.36-7.39 (m, 2H), 7.45 (d, 2H), 7.59 (d, 2H), 8.38 (d, 1H), 9.09 (s, 1H).

### Example 48

### {4-[7-(Oxetan-3-ylmethoxy)quinazolin-4-yl]piperidin-1-yl}[4-(trifluoromethoxy)-phenyl]methanone

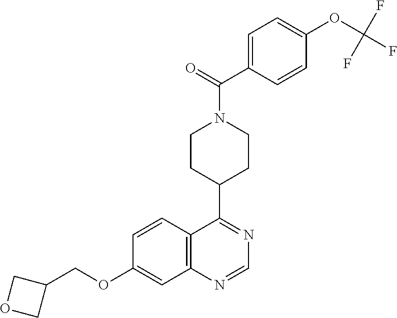 [SMILES: O=C(c1ccc(OC(F)(F)F)cc1)N1CCC(c2ncnc3cc(OCC4COC4)ccc23)CC1]

This compound was synthesized by the same method as described in in example 46 to give 36.5 mg (31%) of the product as a white solid. MS (ESIpos): m/z=488 (M+H)⁺; LC-MS[Method 2]: Rₜ=1.40 min; ¹H-NMR (400 MHz, DMSO-d₆): δ 1.85-1.90 (m, 4H), 3.05-3.07 (m, 1H), 3.28-3.34 (m, 1H), 3.41-3.50 (m, 1H), 3.62-3.67 (m, 1H), 3.98-3.99 (m, 1H), 4.41-4.43 (d, 2H), 4.45-4.48 (m, 2H), 4.61-4.62 (m, 1H), 4.71-4.75 (m, 2H), 7.35-7.45 (m, 4H), 7.56 (d, 2H), 8.37 (d, 1H), 9.08 (s, 1H).

### Example 49

### {4-[7-(Tetrahydro-2H-pyran-4-yloxy)quinazolin-4-yl]piperidin-1-yl}[4-(trifluoromethoxy)phenyl]-methanone

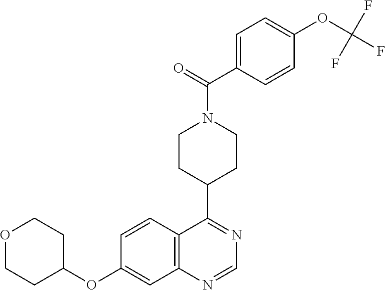 [SMILES: O=C(c1ccc(OC(F)(F)F)cc1)N1CCC(c2ncnc3cc(OC4CCOCC4)ccc23)CC1]

To a solution of [4-(7-Hydroxymethoxyquinazolin-4-yl)piperidin-1-yl][4-(trifluoromethoxy)phenyl]methanone (100 mg, 0.24 mmol) in 1 mL of N,N-dimethyl formamide were added potassium iodide, (41 mg, 0.3 mmol) and 4-(bromomethyl)-tetrahydro-2H-pyran, 119 mg (0.72 mmol). The resulting solution was stirred 12 h at 80° C. After chromatography, the title compound was obtained as a brown solid (24 mg, 26%). After filtration, removal of the solvent and subsequent chromatography, the product was obtained as a white solid in 10% yields (12 mg).

MS (ESIpos): m/z=502 (M+H)⁺; LC-MS[Method 2]: Rₜ=1.52 min; ¹H-NMR (400 MHz, DMSO-d₆): δ 1.63-1.72 (m, 2H), 1.86-1.90 (m, 4H), 2.06-2.09 (m, 2H), 3.07-3.08 (m, 1H), 3.56 (t, 2H), 3.68-3.69 (m, 1H), 3.88 (t, 2H), 3.96-3.99 (m, 1H), 4.61-4.62 (m, 2H), 4.90-4.94 (m, 1H), 7.36 (d, 1H), 7.44-7.46 (m, 3H), 7.59 (d, 2H), 8.38 (d, 1H), 9.09 (s, 1H).

### Example 50

### N-{2-[(4-{1-[4-(Trifluoromethoxy)benzoyl]piperidin-4-yl}quinazolin-7-yl)oxy]ethyl}acetamide

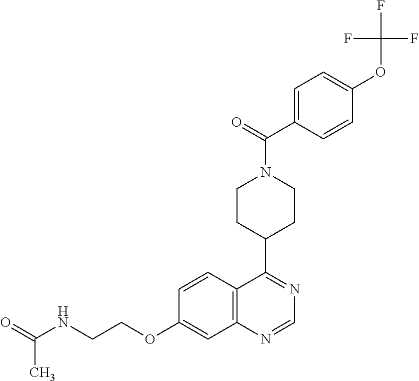 [SMILES: CC(=O)NCCOc1ccc2c(C3CCN(C(=O)c4ccc(OC(F)(F)F)cc4)CC3)ncnc2c1]

### Step 1: {4-[7-(2-Aminoethoxy)quinazolin-4-yl]piperidin-1-yl}[4-(trifluoro-methoxy)phenyl]methanone

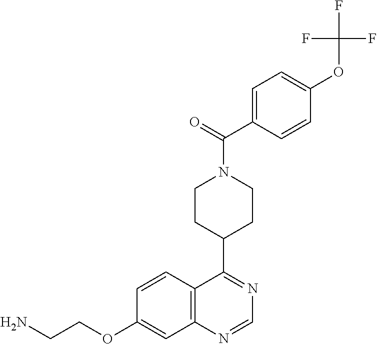 [SMILES: NCCOc1ccc2c(C3CCN(C(=O)c4ccc(OC(F)(F)F)cc4)CC3)ncnc2c1]

To a solution of (4-(7-hydroxyquinazolin-4-yl)piperidin-1-yl)(4-(trifluoromethoxy)phenyl)methanone (100 mg, 0.24 mmol), in 1.0 mL of N,N-dimethylformamide were added tert-butyl 2-iodoethylcarbamate (195 mg, 0.72 mmol), and potassium carbonate (66 mg, 0.48 mmol). The resulting mixture was stirred at 50° C. for 24 hours under nitrogen atmosphere. After cooled to room temperature, the pH value was adjusted to 1 with 1 N hydrochloric acid and the resulting solution was stirred at 50° C. for another 12 hours. After cooled to room temperature, aqueous sodium carbonate was added to adjust the pH value to 7 and the solid was removed by filtration. The filtrate was purified by chromatography to give 40 mg (33%) of the product as a white solid. MS (ESIpos): m/z=461 (M+H)⁺; LC-MS[Method 2]: Rₜ=0.64 min.

### Step 2: N-{2-[(4-{1-[4-(Trifluoromethoxy)benzoyl]piperidin-4-yl}quinazolin-7-yl)oxy]ethyl}acetamide

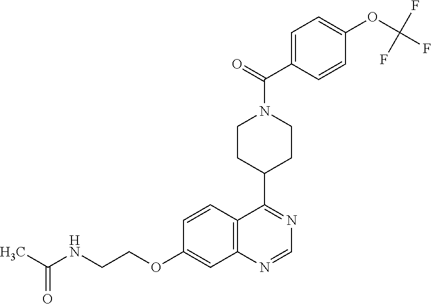 [SMILES: CC(=O)NCCOc1ccc2c(C3CCN(C(=O)c4ccc(OC(F)(F)F)cc4)CC3)ncnc2c1]

To a solution of {4-[7-(2-aminoethoxy)quinazolin-4-yl]piperidin-1-yl}[4-(trifluoromethoxy)phenyl]methanone (40 mg, 0.9 mmol) and triethylamine (26 mg, 0.26 mmol) in 1.0 mL of dichloromethane was added acetyl chloride (10 mg, 0.13 mmol). The resulting mixture was stirred at room temperature for 10 minutes. The solvent was removed in vacuo and the residue was purified by chromatography to give 24.6 mg (55%) of the product as a white solid. MS (ESIpos): m/z=503 (M+H)⁺; LC-MS[Method 2]: Rₜ=1.26 min; ¹H-NMR (400 MHz, DMSO-d₆): δ 1.82-1.95 (m, 7H), 3.05-3.06 (m, 1H), 3.45-3.47 (m, 1H), 3.48-3.50 (t, 2H), 3.67-3.68 (m, 1H), 3.97-3.98 (m, 1H), 4.18-4.21 (t, 2H), 4.61-4.62 (m, 1H), 7.33-7.36 (m, 2H), 7.44 (d, 2H), 7.57 (d, 2H), 8.12-8.15 (m, 1H), 8.37 (d, 1H), 9.08 (s, 1H).

### Example 51

### [4-(7-Cyclopropylquinazolin-4-yl)piperidin-1-yl][4-(trifluoromethoxy)phenyl]-methanone

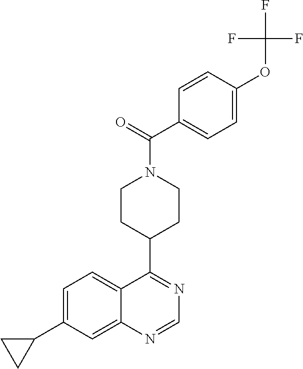 [SMILES: O=C(c1ccc(OC(F)(F)F)cc1)N1CCC(c2ncnc3cc(C4CC4)ccc23)CC1]

### Step 1: [4-{1-[4-(Trifluoromethoxy)benzoyl]piperidin-4-yl}quinazolin-7-yl trifluoromethanesulfonate

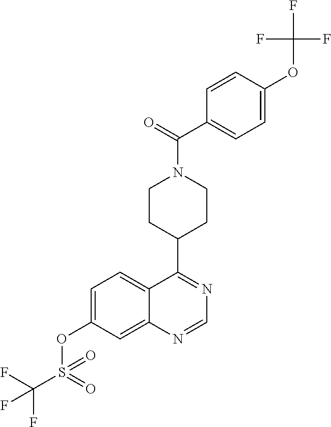 [SMILES: O=C(c1ccc(OC(F)(F)F)cc1)N1CCC(c2ncnc3cc(OS(=O)(=O)C(F)(F)F)ccc23)CC1]

(4-(7-hydroxyquinazolin-4-yl)piperidin-1-yl)(4-(trifluoromethoxy)phenyl)methanone, (1.13 g, 2.7 mmol), pyridine (1.50 g, 19.0 mmol) and trifluoromethanesulfonic anhydride (1.53 g, 5.4 mmol) were dissolved in 30 mL of dichloromethane. The resulting mixture was stirred at room temperature for 20 minutes. The mixture was extracted with dichloromethane and the combined organic phase was dried over anhydrous sodium sulfate. The residue was purified by chromatography to give 1.10 g (69%) of the product as a white solid.

MS(ESIpos): m/z=550 (M+H)⁺.

### Step 2: [4-(7-Cyclopropylquinazolin-4-yl)piperidin-1-yl][4-(trifluoromethoxy)-phenyl]methanone

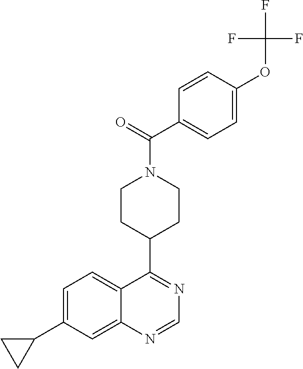 [SMILES: O=C(c1ccc(OC(F)(F)F)cc1)N1CCC(c2ncnc3cc(C4CC4)ccc23)CC1]

To a solution of 4-(1-(4-(trifluoromethoxy)benzoyl)piperidin-4-yl)quinazolin-7-yl trifluoromethanesulfonate (60 mg, 0.1 mmol) in 2 mL of N,N-dimethylformamide were added cyclopropylboronic acid (12 mg, 0.1 mmol), Pd(dppf)Cl₂ (8.0 mg, 0.01 mmol), and potassium carbonate (45 mg, 0.3 mmol). The resulting mixture was stirred at 90° C. for 2 hours under nitrogen atmosphere. After cooled to room temperature, the solid was removed by filtration and the filtrate was purified by chromatography to give 18.5 mg of the product as a light yellow solid. MS (ESIpos): m/z=442 (M+H)⁺; LC-MS [Method 3]: Rₜ=2.04 min. ¹H-NMR (400 MHz, DMSO-d₆): δ 0.90-0.94 (m, 2H), 1.12-1.16 (m, 2H), 1.86-1.91 (m, 4H), 2.20-2.22 (m, 1H), 3.04-3.06 (m, 1H), 3.32-3.36 (m, 1H), 3.68-3.70 (m, 1H), 4.01-4.02 (m, 1H), 4.62-4.65 (m, 1H), 7.44-7.48 (m, 3H), 7.59 (d, 2H), 7.68 (s, 1H), 8.33 (d, 1H), 9.14 (s, 1H).

### Example 52

### {4-[7-(3,6-Dihydro-2H-pyran-4-yl)quinazolin-4-yl]piperidin-1-yl}[4-(trifluoro-methoxy)phenyl]-methanone

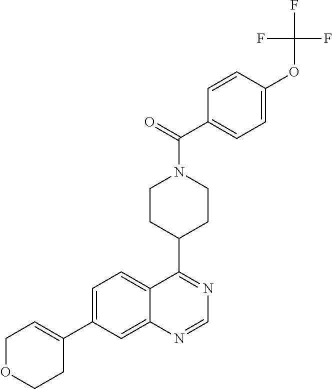 [SMILES: O=C(c1ccc(OC(F)(F)F)cc1)N1CCC(c2ncnc3cc(C4=CCOCC4)ccc23)CC1]

This compound was synthesized by the same method as described in example 51 to give 22.6 mg (43%) of the product as a light yellow solid. MS (ESIpos): m/z=484 (M+H)⁺; LC-MS [Method 3]: Rₜ=1.93 min. ¹H-NMR (400 MHz, DMSO-d₆): δ 1.87-1.92 (m, 4H), 2.61-2.63 (m, 2H), 3.09-3.11 (m, 1H), 3.33-3.36 (m, 1H), 3.69-3.71 (m, 1H), 3.87-3.90 (m, 2H), 4.06-4.08 (m, 1H), 4.32-4.33 (m, 2H), 4.63-4.65 (m, 1H), 6.70-6.71 (t, 1H), 7.46 (d, 2H), 7.59 (d, 2H), 7.93 (s, 1H), 7.95 (d, 1H), 8.42 (d, 1H), 9.19 (s, 1H).

### Example 53

### {4-[7-(1H-Pyrazol-5-yl)quinazolin-4-yl]piperidin-1-yl}[4-(trifluoromethoxy)-phenyl]methanone

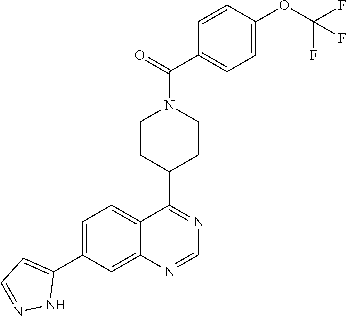 [SMILES: O=C(c1ccc(OC(F)(F)F)cc1)N1CCC(c2ncnc3cc(-c4ccn[nH]4)ccc23)CC1]

This compound was synthesized by the same method as described in in example 51 to give 40 mg (46%) of the title compound as a white solid. MS (ESIpos): m/z=468 (M+H)⁺; LC-MS [Method 2]: Rₜ=1.39 min. ¹H-NMR (400 MHz, DMSO-d₆): δ 1.89-1.91 (m, 4H), 3.11-3.12 (m, 1H), 3.33-3.39 (m, 1H), 3.70-3.71 (m, 1H), 4.07-4.08 (m, 1H), 4.64-4.65 (m, 1H), 7.07 (s, 1H), 7.46 (d, 2H), 7.60 (d, 2H), 7.90-7.91 (m, 1H), 8.27-8.29 (m, 1H), 8.38 (s, 1H), 8.50 (d, 1H), 9.20 (s, 1H), 13.20 (br, 1H).

### Example 54

### 1-(4-{1-[4-(Trifluoromethoxy)benzoyl]piperidin-4-yl}quinazolin-7-yl)pyrrolidin-2-one

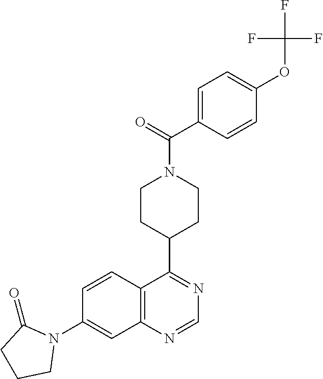 [SMILES: O=C(c1ccc(OC(F)(F)F)cc1)N1CCC(c2ncnc3cc(N4CCCC4=O)ccc23)CC1]

4-(1-(4-(Trifluoromethoxy)benzoyl)piperidin-4-yl)quinazolin-7-yl trifluoromethanesulfonate (60 mg, 0.1 mmol), pyrrolidin-2-one (11 mg, 0.1 mmol), tris(dibenzylideneacetone)dipalladium (10 mg, 0.01 mmol), Xantphos (19 mg, 0.03 mmol), and cesium carbonate (107 mg, 0.3 mmol) were added to 2 mL of 1,4-dioxane. The resulting mixture was stirred at 100° C. for 15 hours under nitrogen atmosphere. After cooled to room temperature, the solid was removed by filtration and the filtrate was purified by chromatography to give 37.3 mg (69%) of the product as a light yellow solid. MS (ESIpos): m/z=485 (M+H)⁺; LC-MS [Method 2]: Rₜ=2.92 min. ¹H-NMR (400 MHz, DMSO-d₆): δ 1.87-1.89 (m, 4H), 2.11-2.15 (m, 2H), 2.59-2.63 (m, 2H), 3.06-3.08 (m, 1H), 3.33-3.36 (m, 1H), 3.66-3.74 (m, 1H), 4.00-4.03 (m, 3H), 4.64-4.66 (m, 1H), 7.46 (d, 2H), 7.59 (d, 2H), 8.03 (s, 1H), 8.32 (d, 1H), 8.46 (d, 1H), 9.15 (s, 1H).

### Example 55

### 1-(4-{1-[4-(Trifluoromethoxy)benzoyl]piperidin-4-yl}quinazolin-7-yl)piperidin-2-one

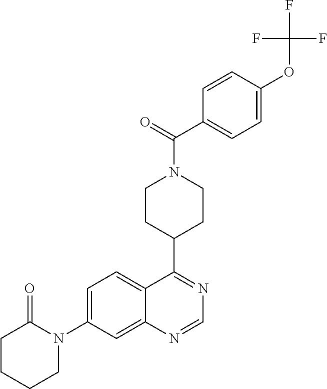 [SMILES: O=C(c1ccc(OC(F)(F)F)cc1)N1CCC(c2ncnc3cc(N4CCCCC4=O)ccc23)CC1]

This compound was synthesized by the same method as described in example 54 to give 36 mg (66%) of the product as a light yellow solid. MS (ESIpos): m/z=499 (M+H)⁺; LC-MS [Method 3]: Rₜ=1.72 min. ¹H-NMR (400 MHz, DMSO-d₆): δ 1.84-1.92 (m, 8H), 2.45-2.47 (m, 2H), 3.08-3.12 (m, 1H), 3.33-3.42 (m, 1H), 3.66-3.74 (m, 1H), 3.80-3.82 (m, 2H), 4.01-4.05 (m, 1H), 4.62-4.68 (m, 1H), 7.45 (d, 2H), 7.61 (d, 2H), 7.76 (d, 1H), 7.87 (s, 1H), 8.43 (d, 1H), 9.18 (s, 1H).

### Example 56

### Formic Acid—(4-{7-[2-(dimethylamino)ethoxy]quinazolin-4-yl}piperidin-1-yl)[4-(trifluoromethoxy)phenyl]methanone (1:1)

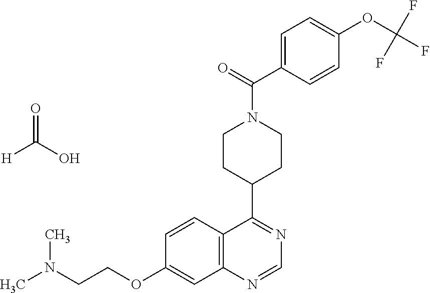 [SMILES: O=CO ; CN(C)CCOc1ccc2c(C3CCN(C(=O)c4ccc(OC(F)(F)F)cc4)CC3)ncnc2c1]

This compound was synthesized by the same method as described in in example 46 to afford the desired product in 28% yields (30 mg).

MS (ESIpos): m/z=489 (M+H)⁺; LC-MS [Method 2]: Rₜ=1.69 min; ¹H-NMR (400 MHz, DMSO-d₆): δ 1.86-1.91 (m, 4H), 2.26 (s, 6H), 2.67 (t, 2H), 3.05-3.15 (m, 1H), 3.30-3.41 (m, 1H), 3.68-3.69 (m, 1H), 3.98-4.02 (m, 1H), 4.28 (t, 2H), 4.63-4.65 (m, 1H), 7.35-7.39 (m, 2H), 7.46 (d, 2H), 7.59 (d, 2H), 8.17 (s, 1H), 8.37 (d, 1H), 9.10 (s, 1H).

### Example 57

### [4-(6-Methoxyquinazolin-4-yl)piperidin-1-yl][4-(trifluoromethoxy)phenyl]-methanone

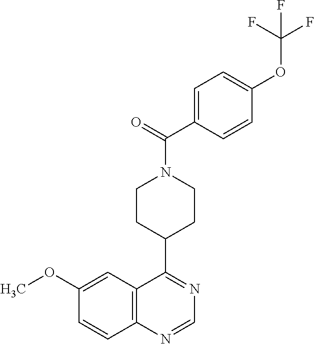 [SMILES: COc1ccc2ncnc(C3CCN(C(=O)c4ccc(OC(F)(F)F)cc4)CC3)c2c1]

### Step 1: 6-Methoxyquinazolin-4(3H)-one

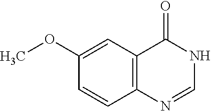

This compound was synthesized by the same method as described in in example 1 to give 2.09 g (65%) of the product as a brown solid. MS (ESIpos): m/z=177 (M+H)⁺; LC-MS [Method 2]: Rₜ=0.59 min.

### Step 2: 4-Chloro-6-methoxyquinazoline

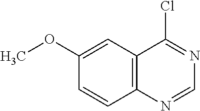

This compound was synthesized by the same method as described in in example 1 to give 2.12 g (51%) of the product as a brown solid. MS (ESIpos): m/z=195 (M+H)⁺; LC-MS [Method 2]: Rₜ=0.92 min.

### Step 3: Tert-butyl 4-(6-methoxyquinazolin-4-yl)-5,6-dihydropyridine-1(2H)-carboxylate

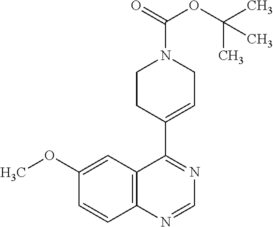 [SMILES: COc1ccc2ncnc(C3=CCN(C(=O)OC(C)(C)C)CC3)c2c1]

This compound was synthesized by the same method as described in in example 1 to give 1.9 g (94%) of the product as a light yellow solid. MS (ESIpos): m/z=342 (M+H)⁺; LC-MS [Method 1]: Rₜ=2.58 min. ¹H-NMR (400 MHz, DMSO-d₆): δ 1.46 (s, 9H), 2.67-2.68 (m, 2H), 3.64 (t, 2H), 3.94 (s, 3H), 4.14-4.15 (m, 2H), 6.37 (br, 1H), 7.53 (s, 1H), 7.66-7.69 (m, 1H), 7.96 (d, 1H), 9.10 (s, 1H).

### Step 4: 6-Methoxy-4-(1,2,3,6-tetrahydropyridin-4-yl)quinazoline

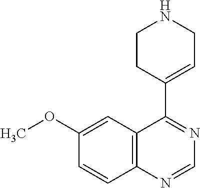 [SMILES: COc1ccc2ncnc(C3=CCNCC3)c2c1]

This compound was synthesized by the same method as described in in example 1 to give 0.83 g (75%) of the product as a yellow solid. MS (ESIpos): m/z=242 (M+H)⁺; LC-MS [Method 1]: Rₜ=0.69 min. ¹H-NMR (400 MHz, DMSO-d₆): δ 2.51-2.55 (m, 2H), 2.95-3.05 (m, 2H), 3.60-3.75 (m, 2H), 3.94 (s, 3H), 4.15-4.20 (m, 1H), 6.35 (br, 1H), 7.55 (s, 1H), 7.65-7.68 (m, 1H), 7.95 (d, 1H), 9.09 (s, 1H).

### Step 5: 6-Methoxy-4-(piperidin-4-yl)quinazoline

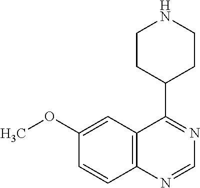 [SMILES: COc1ccc2ncnc(C3CCNCC3)c2c1]

6-Methoxy-4-(1,2,3,6-tetrahydropyridin-4-yl)quinazoline (0.40 g, 1.7 mmol), was dissolved in 20 mL of methanol. Palladium hydroxide/carbon (10%) (0.12 g, 0.17 mmol), was added and the resulting mixture was stirred at room temperature for 30 minutes under hydrogen atmosphere (3 atm). After filtration, the filtrate was concentrated in vacuo to give 0.37 g (91%) of the product as an off-white solid. MS (ESIpos): m/z=244 (M+H)⁺; LC-MS [Method 2]: Rₜ=0.72 min. ¹H-NMR (400 MHz, DMSO-d₆): δ 1.76-1.85 (m, 4H), 2.75-2.82 (m, 2H), 3.04-3.07 (m, 2H), 3.73-3.79 (m, 1H), 3.98 (s, 3H), 7.57 (s, 1H), 7.62-7.65 (m, 1H), 7.93 (d, 1H), 9.08 (s, 1H).

### Step 6: (4-(6-Methoxyquinazolin-4-yl)piperidin-1-yl)(4-(trifluoromethoxy)phenyl)-methanone

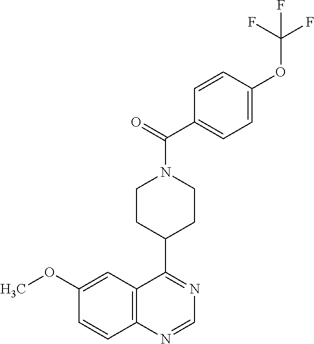 [SMILES: COc1ccc2ncnc(C3CCN(C(=O)c4ccc(OC(F)(F)F)cc4)CC3)c2c1]

This compound was synthesized by the same method as described in in example 9 to give 34.4 mg (19%) of the product as an off-white solid. MS (ESIpos): m/z=432 (M+H)⁺; LC-MS [Method 2]: Rₜ=1.51 min. ¹H-NMR (400 MHz, DMSO-d₆): δ 1.86-1.99 (m, 4H), 3.13-3.15 (m, 1H), 3.40-3.42 (m, 1H), 3.69-3.75 (m, 1H), 3.99 (s, 3H), 4.03-4.10 (m, 1H), 4.62-4.68 (m, 1H), 7.47 (d, 2H), 7.60 (d, 2H), 7.65-7.68 (m, 2H), 7.95 (d, 1H), 9.10 (s, 1H).

### Example 58

### [4-(Quinazolin-4-yl)piperidin-1-yl][4-(trifluoromethoxy)phenyl]methanone

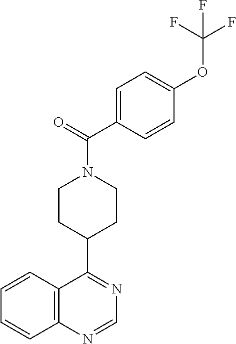 [SMILES: O=C(c1ccc(OC(F)(F)F)cc1)N1CCC(c2ncnc3ccccc23)CC1]

### Step 1: Quinazolin-4(3H)-one

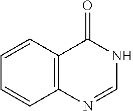 [SMILES: O=c1[nH]cnc2ccccc12]

This compound was synthesized by the same method as described in example 1 to give 1.25 g (56%) of the product as a brown solid. MS (ESIpos): m/z=147 (M+H)⁺; LC-MS [Method 1]: Rₜ=0.53 min.

### Step 2: 4-Chloroquinazoline

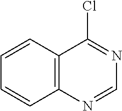 [SMILES: Clc1ncnc2ccccc12]

This compound was synthesized by the same method as described in example 1 to give 1.33 g (crude) of the product as a brown solid. MS (ESIpos): m/z=165 (M+H)⁺; LC-MS [Method 1]: Rₜ=0.87 min.

### Step 3: Tert-butyl 4-(quinazolin-4-yl)-5,6-dihydropyridine-1(2H)-carboxylate

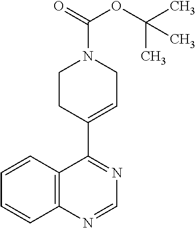 [SMILES: CC(C)(C)OC(=O)N1CC=C(c2ncnc3ccccc23)CC1]

This compound was synthesized by the same method as described in example 1 to give 2.51 g (crude) of the product as a light yellow semi-solid. MS (ESIpos): m/z=312 (M+H)⁺; LC-MS [Method 1]: Rₜ=1.77 min. ¹H-NMR (400 MHz, DMSO-d₆): δ 1.46 (s, 9H), 2.67-2.68 (m, 2H), 3.64 (t, 2H), 4.15-4.17 (m, 2H), 6.28 (br, 1H), 7.73-7.76 (m, 1H), 8.02-8.04 (m, 2H), 8.33 (d, 1H), 9.24 (s, 1H).

### Step 4: 4-(1,2,3,6-Tetrahydropyridin-4-yl)quinazoline

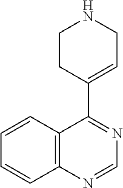 [SMILES: C1=C(c2ncnc3ccccc23)CCNC1]

This compound was synthesized by the same method as described in example 1 to give 0.65 g (77%) of the product as yellow oil. MS (ESIpos): m/z=212 (M+H)⁺; LC-MS [Method 2]: Rₜ=0.56 min. ¹H-NMR (400 MHz, DMSO-d₆): δ 2.54-2.67 (m, 2H), 2.99-3.01 (m, 2H), 3.49-3.51 (m, 2H), 4.17 (br, 1H), 6.27 (br, 1H), 7.70-7.77 (m, 1H), 7.99-8.03 (m, 2H), 8.32 (d, 1H), 9.22 (s, 1H).

### Step 5: 4-(Piperidin-4-yl)quinazoline

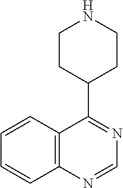 [SMILES: c1ccc2c(C3CCNCC3)ncnc2c1]

This compound was synthesized by the same method as described in example 57 to give 0.30 g (84%) of the product as an off-white solid. MS (ESIpos): m/z=214 (M+H)⁺; LC-MS [Method 1]: Rₜ=0.73 min. ¹H-NMR (400 MHz, DMSO-d₆): δ 1.70-1.80 (m, 4H), 2.24-2.30 (m, 1H), 2.66-2.72 (m, 2H), 2.98-3.01 (m, 2H), 3.67-3.75 (m, 1H), 7.66-7.72 (m, 1H), 7.90-7.95 (m, 2H), 8.34 (d, 1H), 9.15 (s, 1H).

### Step 6: (4-(Quinazolin-4-yl)piperidin-1-yl)(4-(trifluoromethoxy)phenyl)-methanone

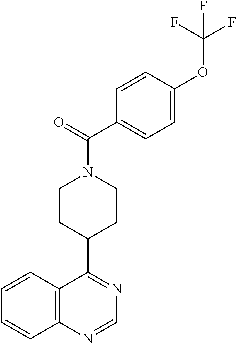 [SMILES: O=C(c1ccc(OC(F)(F)F)cc1)N1CCC(c2ncnc3ccccc23)CC1]

This compound was synthesized by the same method as described in example 9 to give 47.5 mg (25%) of the product as an off-white solid. MS (ESIpos): m/z=402 (M+H)⁺; LC-MS [Method 3]: Rₜ=1.79 min. ¹H-NMR (400 MHz, DMSO-d₆): δ 1.88-1.99 (m, 4H), 3.09-3.11 (m, 1H), 3.34-3.39 (m, 1H), 3.69-3.72 (m, 1H), 4.05-4.13 (m, 1H), 4.64-4.70 (m, 1H), 7.46 (d, 2H), 7.59-7.61 (m, 2H), 7.76-7.81 (m, 1H), 7.99-8.04 (m, 2H), 8.49 (d, 1H), 9.23 (s, 1H).

### Example 59

### [4-(8-Methylquinazolin-4-yl)piperidin-1-yl][4-(trifluoromethoxy)phenyl]-methanone

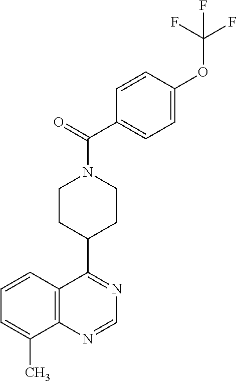 [SMILES: Cc1cccc2c(C3CCN(C(=O)c4ccc(OC(F)(F)F)cc4)CC3)ncnc12]

### Step 1: 8-Methylquinazolin-4(3H)-one

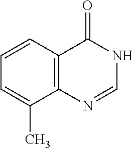

This compound was synthesized by the same method as described in example 1 to give 1.48 g (92%) of the product as a brown solid. MS (ESIpos): m/z=161 (M+H)⁺; LC-MS [Method 1]: Rₜ=0.66 min.

### Step 2: 4-Chloro-8-methylquinazoline

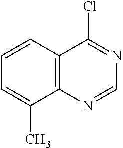

This compound was synthesized by the same method as described in example 1 to give 1.51 g (89%) of the product as a brown solid. MS (ESIpos): m/z=179 (M+H)⁺; LC-MS [Method 2]: Rₜ=1.02 min.

### Step 3: Tert-butyl 4-(8-methylquinazolin-4-yl)-5,6-dihydropyridine-1(2H)-carboxylate

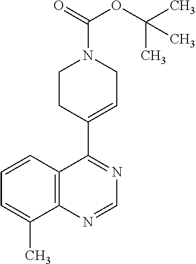 [SMILES: Cc1cccc2c(C3=CCN(C(=O)OC(C)(C)C)CC3)ncnc12]

This compound was synthesized by the same method as described in example 1 to give 2.53 g (92%) of the product as a light yellow semi-solid. MS (ESIpos): m/z=326 (M+H)⁺; LC-MS [Method 2]: Rₜ=1.62 min. ¹H-NMR (400 MHz, CD₃OD): δ 1.54 (s, 9H), 2.72-2.75 (m, 2H), 2.78 (s, 3H), 3.77-3.79 (m, 2H), 4.23-4.25 (m, 2H), 6.21 (br, 1H), 7.60-7.64 (m, 1H), 7.87 (d, 1H), 8.15 (d, 1H), 9.20 (s, 1H).

### Step 4: 8-Methyl-4-(1,2,3,6-tetrahydropyridin-4-yl)quinazoline

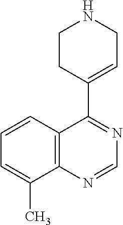 [SMILES: Cc1cccc2c(C3=CCNCC3)ncnc12]

This compound was synthesized by the same method as described in example 1 to give 0.65 g (94%) of the product as an off-white solid. MS (ESIpos): m/z=226 (M+H)⁺; LC-MS [Method 2]: Rₜ=0.75 min. ¹H-NMR (400 MHz, DMSO-d₆): δ 2.51-2.67 (m, 2H), 2.71 (s, 3H), 2.99-3.02 (m, 1H), 3.50-3.76 (m, 3H), 4.15-4.17 (m, 1H), 6.21 (s, 1H), 7.61 (t, 1H), 7.86 (d, 1H), 8.14 (d, 1H), 9.25 (s, 1H).

### Step 5: 8-Methyl-4-(piperidin-4-yl)quinazoline

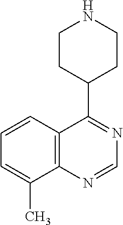 [SMILES: Cc1cccc2c(C3CCNCC3)ncnc12]

This compound was synthesized by the same method as described in example 57 to give 0.20 g (79%) of the product as an off-white solid. MS (ESIpos): m/z=228 (M+H)⁺; LC-MS [Method 3]: Rₜ=0.95 min. ¹H-NMR (400 MHz, DMSO-d₆): δ 1.73-1.76 (m, 4H), 2.63 (s, 3H), 2.72-2.78 (m, 1H), 2.94-2.98 (m, 2H), 3.63-3.82 (m, 2H), 4.03-4.06 (m, 1H), 7.57 (t, 1H), 7.78 (d, 1H), 8.20 (d, 1H), 9.17 (s, 1H).

### Step 6: (4-(8-Methylquinazolin-4-yl)piperidin-1-yl)(4-(trifluoromethoxy)phenyl)-methanone

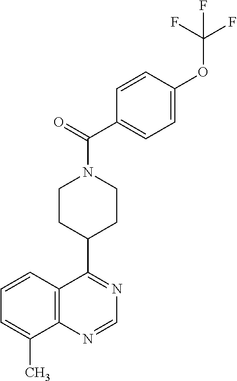 [SMILES: Cc1cccc2c(C3CCN(C(=O)c4ccc(OC(F)(F)F)cc4)CC3)ncnc12]

This compound was synthesized by the same method as described in example 9 to give 52.6 mg (28%) of the product as an off-white solid. MS (ESIpos): m/z=416 (M+H)⁺; LC-MS [Method 2]: Rₜ=1.62 min. ¹H-NMR (400 MHz, DMSO-d₆): δ 1.88-1.98 (m, 4H), 2.70 (s, 3H), 3.08-3.12 (m, 1H), 3.32-3.35 (m, 1H), 3.68-3.72 (m, 1H), 4.04-4.12 (m, 1H), 4.63-4.66 (m, 1H), 7.46 (d, 2H), 7.58-7.62 (m, 2H), 7.64-7.68 (m, 1H), 7.87 (d, 1H), 8.32 (d, 1H), 9.26 (s, 1H).

### Example 60

### [4-(7-Methoxyquinolin-4-yl)piperidin-1-yl][4-(trifluoromethoxy)-phenyl]methanone

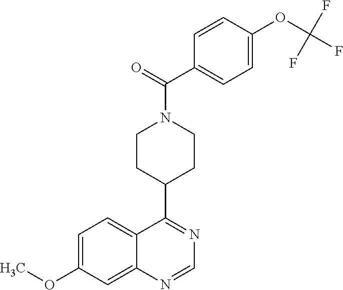 [SMILES: COc1ccc2c(C3CCN(C(=O)c4ccc(OC(F)(F)F)cc4)CC3)ncnc2c1]

This compound was synthesized by the same method as described in example 9 to give 169 mg of the title compound as a white solid. MS (ESIpos): m/z=431 (M+H)⁺; LC-MS [Method 2]: Rₜ=1.21 min. ¹H-NMR (400 MHz, DMSO-d₆): δ 1.72-1.80 (m, 4H), 3.06-3.07 (m, 1H), 3.30-3.37 (m, 1H), 3.69-3.73 (m, 2H), 3.92 (s, 3H), 4.67-4.68 (m, 1H), 7.26-7.29 (m, 1H), 7.33 (d, 1H), 7.41 (d, 1H), 7.46 (d, 2H), 7.62 (d, 2H), 8.22 (d, 1H), 8.76 (d, 1H).

### Example 61

### [4-(7-Methoxyquinazolin-4-yl)piperidin-1-yl][2-methyl-4-(trifluoromethoxy)-phenyl]methanone

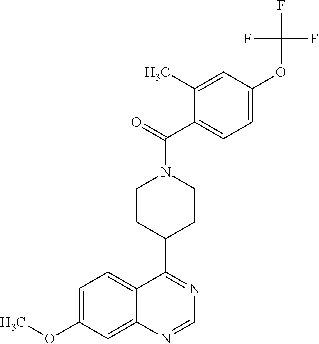 [SMILES: COc1ccc2c(C3CCN(C(=O)c4ccc(OC(F)(F)F)cc4C)CC3)ncnc2c1]

To a mixture of 7-methoxy-4-(piperidin-4-yl)quinazoline (100 mg, 0.45 mmol) and triethylamine (130 mg, 1.2 mmol) in 2 mL dichloromethane were added 2-methyl-4-(trifluoromethoxy)benzoic acid (100 mg, 0.41 mmol) and TBTU (160 mg, 0.5 mmol. The resulting mixture was stirred at temperature for 2.5 hours. The solvent was removed in vacuo and the residue was purified by chromatography to give 61 mg (32%) of the product as a white solid.

MS (ESIpos): m/z=446 (M+H)⁺; LC-MS [Method 2]: Rₜ=2.65 min. ¹H-NMR (400 MHz, DMSO-d₆): δ 1.75-1.98 (m, 4H), 2.27 (s, 3H), 3.04-3.10 (m, 1H), 3.31-3.38 (m, 2H), 3.96 (s, 3H), 4.09-4.10 (m, 1H), 4.70-4.71 (m, 1H), 7.25-7.36 (m, 5H), 8.37 (d, 1H), 9.11 (s, 1H).

### Example 62

### [4-(7-Methoxyquinazolin-4-yl)piperidin-1-yl][3-methyl-4-(trifluoromethoxy)-phenyl]methanone

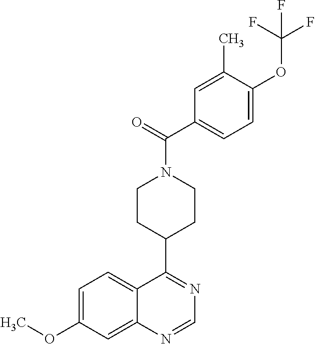 [SMILES: COc1ccc2c(C3CCN(C(=O)c4ccc(OC(F)(F)F)c(C)c4)CC3)ncnc2c1]

(3-Bromo-4-(trifluoromethoxy)phenyl)(4-(7-methoxyquinazolin-4-yl)piperidin-1-yl)methanone (60 mg, 0.1 mmol), methylboronic acid (25 mg, 0.4 mmol), sodium carbonate (55 mg, 0.5 mmol), and Pd(dppf)Cl₂ (20 mg, 0.02 mmol), were combined in 5 mL of 1,4-dioxane/water (v:v=4:1). The resulting mixture was stirred at 100° C. for 20 hours under nitrogen atmosphere. After cooled to room temperature, the solvent was removed in vacuo and the residue was re-dissolved with water. The resulting solution was extracted with ethyl acetate and the combined organic layer was dried over anhydrous sodium sulfate. The solvent was removed in vacuo and the residue was purified by chromatography to give 17.8 mg (38%) of the product as a white solid. MS (ESIpos): m/z=446 (M+H)⁺; LC-MS [Method 2]: Rₜ=1.51 min. ¹H-NMR (400 MHz, CD₃OD): δ 1.85-1.92 (m, 1H), 2.08-2.10 (m, 3H), 2.40 (s, 3H), 3.10-3.17 (m, 1H), 3.44-3.45 (m, 1H), 3.88-3.91 (m, 1H), 4.00 (s, 3H), 4.02-4.03 (m, 1H), 4.81-4.84 (m, 1H), 7.35-7.44 (m, 4H), 7.49 (s, 1H), 8.37 (d, 1H), 9.07 (s, 1H).

### Example 63

### {4-[7-(2-Hydroxy-2-methylpropoxy)quinazolin-4-yl]piperidin-1-yl}[4-(trifluoro-methoxy) phenyl]-methanone

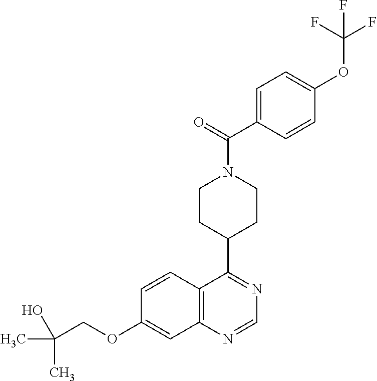 [SMILES: CC(C)(O)COc1ccc2c(C3CCN(C(=O)c4ccc(OC(F)(F)F)cc4)CC3)ncnc2c1]

To a solution of (4-(7-hydroxyquinazolin-4-yl)piperidin-1-yl)(4-(trifluoromethoxy)-phenyl)methanone (150 mg, 0.36 mmol) in 4 mL of 1,2-dimethoxyethane and 1 mL of water were added 2,2-dimethyloxirane (518 mg, 7.19 mmol) and sodium hydroxide (29 mg, 0.72 mmol). The resulting mixture was stirred at 90° C. for 20 hours. 1N HCl solution was added to adjust the pH (pH=7). The solvent was removed in vacuo and the residue was purified by chromatography to afford 39 mg (22%) of the title compound as a white solid.

MS (ESIpos): m/z=490 (M+H)⁺; LC-MS [Method 2]: Rₜ=1.44 min. ¹H-NMR (400 MHz, DMSO-d₆): δ 1.25 (s, 6H), 1.83-1.89 (m, 4H), 3.08-3.09 (m, 1H), 3.35-3.40 (m, 1H), 3.68-3.69 (m, 1H), 3.94 (s, 2H), 3.97-4.00 (m, 1H), 4.63-4.64 (m, 1H), 4.74 (s, 1H), 7.33 (d, 1H), 7.39 (d, 1H), 7.45-7.49 (m, 2H), 7.58-7.60 (m, 2H), 8.38 (d, 1H), 9.10 (s, 1H).

### Example 64

### {4-[7-(2,2-Difluoroethoxy)quinazolin-4-yl]piperidin-1-yl}[4-(trifluoromethoxy)-phenyl]methanone

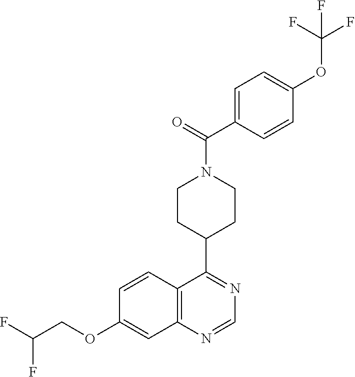 [SMILES: O=C(c1ccc(OC(F)(F)F)cc1)N1CCC(c2ncnc3cc(OCC(F)F)ccc23)CC1]

This compound was synthesized by the same method as described in example 46. The mixture was stirred for 2 h at 80° C. to give 31 mg (33%) of the title compound as a white solid after chromatography. MS (ESIpos): m/z=482 (M+H)⁺; LC-MS [Method 1]: Rₜ=1.57 min. ¹H-NMR (400 MHz, DMSO-d₆): δ 1.84-1.92 (m, 4H), 3.08-3.09 (m, 1H), 3.35-3.40 (m, 1H), 3.68-3.69 (m, 1H), 4.02-4.03 (m, 1H), 4.54-4.62 (m, 3H), 6.49 (t, 1H), 7.43-7.49 (m, 4H), 7.59 (d, 2H), 8.43 (d, 1H), 9.14 (s, 1H).

### Example 65

### [3-Amino-4-(trifluoromethoxy)phenyl][4-(7-methoxyquinazolin-4-yl)piperidin-1-yl]methanone

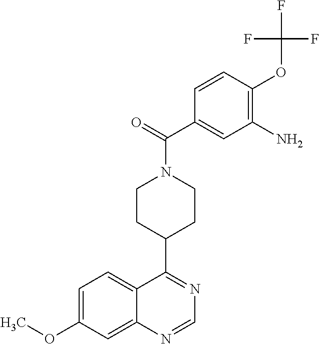 [SMILES: COc1ccc2c(C3CCN(C(=O)c4ccc(OC(F)(F)F)c(N)c4)CC3)ncnc2c1]

### Step 1: 3-Nitro-4-(trifluoromethoxy) benzoic Acid

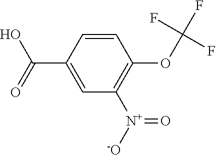

To a solution of 4-(trifluoromethoxy) benzoic acid (1.64 g, 8.0 mmol) in 15 mL of concentrated sulfuric acid was added 10 mL of nitric acid/sulfuric acid (v:v=1:1) dropwise at 0° C. The resulting mixture was stirred at room temperature for 30 min. Upon completion of the reaction, the mixture was poured into ice cold water and the precipitated solid was collected by filtration. The filter cake was washed with water and petroleum ether, dried in vacuo to give 1.81 g (91%) of the product as a white solid.

¹H-NMR (400 MHz, DMSO-d₆): δ 7.88 (d, 1H), 8.35 (d, 1H), 8.59 (s, 1H), 13.86 (br, 1H).

### Step 2: (4-(7-Methoxyquinazolin-4-yl) piperidin-1-yl) (3-nitro-4-(trifluoro-methoxy)phenyl) methanone

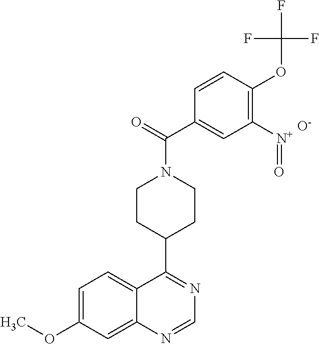

This compound was synthesized by the same method as described in example 1 to give 365 mg (57%) of the product as a yellow solid.

MS (ESIpos): m/z=477 (M+H)⁺; LC-MS [Method 2]: Rₜ=1.05 min.

### Step 3: (3-Amino-4-(trifluoromethoxy)phenyl)(4-(7-methoxyquinazolin-4-yl)piperidin-1-yl)methanone

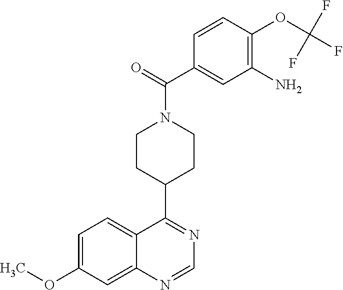 [SMILES: COc1ccc2c(C3CCN(C(=O)c4ccc(OC(F)(F)F)c(N)c4)CC3)ncnc2c1]

This compound was synthesized by the same method as described in example 1 to give 130 mg (85%) of the product as a white solid. MS (ESIpos): m/z=447 (M+H)⁺; LC-MS [Method 2]: Rₜ=1.33 min. ¹H-NMR (400 MHz, DMSO-d₆): δ 1.75-1.86 (m, 4H), 2.93-3.06 (m, 1H), 3.23-3.35 (m, 1H), 3.79-3.84 (m, 1H), 3.97 (s, 3H), 3.98-4.08 (m, 1H), 4.52-4.63 (m, 1H), 5.61 (br, 2H), 6.58 (d, 1H), 6.86 (s, 1H), 7.15 (d, 1H), 7.35-7.38 (m, 2H), 8.38 (d, 1H), 9.11 (s, 1H).

### Example 66

### [2-Amino-4-(trifluoromethoxy)phenyl][4-(7-methoxyquinazolin-4-yl)piperidin-1-yl]methanone

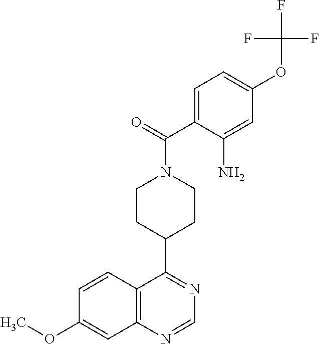 [SMILES: COc1ccc2c(C3CCN(C(=O)c4ccc(OC(F)(F)F)cc4N)CC3)ncnc2c1]

### Step 1: Tert-butyl N-[2-bromo-5-(trifluoromethoxy)phenyl]-N-[(tert-butoxy) carbonyl]carbamate

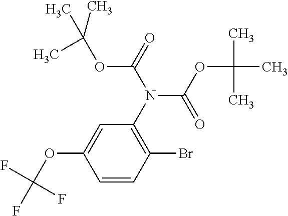 [SMILES: CC(C)(C)OC(=O)N(C(=O)OC(C)(C)C)c1cc(OC(F)(F)F)ccc1Br]

To a solution of 2-bromo-5-(trifluoromethoxy)benzenamine (1.0 g, 3.9 mmol) in 20 mL of tetrahydrofuran were added N,N-dimethylpyridin-4-amine (50 mg, 0.4 mmol), and di-tert-butyl dicarbonate (2.55 g, 11.7 mmol). The resulting mixture was stirred at reflux for overnight. After cooled to room temperature, the solvent was removed in vacuo and the residue was purified by silica gel column chromatography (petroleum ether:ethyl acetate=10:1) to give 1.4 g (80%) of the product as a white solid.

MS (ESIpos): m/z=456 (M+H)⁺; LC-MS [Method 2]: Rₜ=1.07 min.

### Step 2: Ethyl 2-(tert-butoxycarbonylamino)-4-(trifluoromethoxy) benzoate

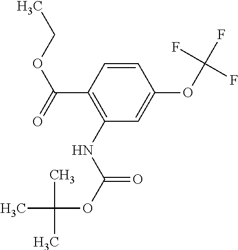 [SMILES: CCOC(=O)c1ccc(OC(F)(F)F)cc1NC(=O)OC(C)(C)C]

Tert-butyl N-[2-bromo-5-(trifluoromethoxy)phenyl]-N-[(tert-butoxy) carbonyl]carbamate (500 mg, 1.1 mmol), trimethylamine (333 mg, 3.3 mmol) and 1,1′-bis(diphenylphosphino)ferrocenepalladium(II) chloride (80 mg, 0.1 mmol), were combined in 15 mL of ethanol under nitrogen. The reaction mixture was stirred 12 hours at 120° C. under CO atmosphere (50 atm). After evaporation in vacuo, the residue was subjected to column chromatography (petroleum ether/ethyl acetate=5:1) to yield 356 mg (93%) of the product, as a colorless oil.

MS (ESIpos): m/z=250 (M+H-100)⁺; LC-MS [Method 2]: Rₜ=1.43 min.

### Step 3: 2-(Tert-butoxycarbonylamino)-4-(trifluoromethoxy) benzoic Acid

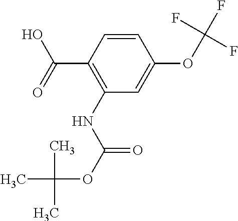 [SMILES: CC(C)(C)OC(=O)Nc1cc(OC(F)(F)F)ccc1C(=O)O]

Ethyl 2-(tert-butoxycarbonylamino)-4-(trifluoromethoxy) benzoate (356 mg, 1.0 mmol), and sodium hydroxide, 80.0 mg (80 mg, 8.0 mmol), were dissolved in 18 mL of water/ethanol (v:v=1:5). The resulting mixture was stirred at room temperature for 2 hours. The organic solvent was removed in vacuo and the residue was diluted with water. Hydrochloric acid solution (1M) was added to the above solution to adjust the pH value to 5. The precipitate was collected by filtration and the filter cake was dried to give 278 mg (84%) of the product as a white solid.

MS (ESIpos): m/z=222 (M+H-Boc)⁺; LC-MS [Method 2]: Rₜ=1.25 min.

### Step 4: Tert-butyl 2-(4-(7-methoxyquinazolin-4-yl) piperidine-1-carbonyl)-5-(trifluoromethoxy) phenylcarbamate

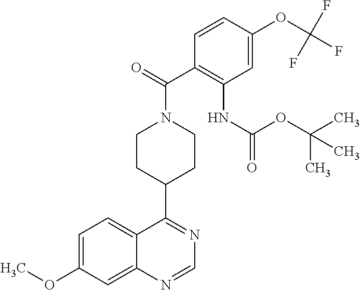 [SMILES: COc1ccc2c(C3CCN(C(=O)c4ccc(OC(F)(F)F)cc4NC(=O)OC(C)(C)C)CC3)ncnc2c1]

This compound was synthesized by the same method as described in example 1 to give 196 mg (68%) of the product as a yellow solid.

MS (ESIpos): m/z=547 (M+H)⁺; LC-MS [Method 2]: Rₜ=1.25 min.

### Step 5: (2-Amino-4-(trifluoromethoxy)phenyl) (4-(7-methoxyquinazolin-4-yl) piperidin-1-yl) methanone

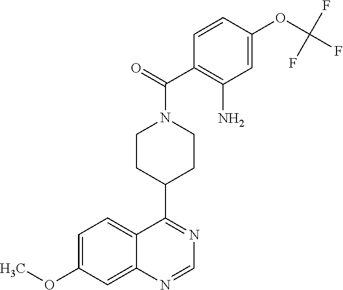 [SMILES: COc1ccc2c(C3CCN(C(=O)c4ccc(OC(F)(F)F)cc4N)CC3)ncnc2c1]

This compound was synthesized by the same method as described in example 1 to give 148 mg (92%) of the product as a yellow solid.

MS (ESIpos): m/z=447 (M+H)⁺; LC-MS [Method 2]: Rₜ=1.48 min. ¹H-NMR (400 MHz, DMSO-d₆): δ 1.79-1.87 (m, 4H), 3.09-3.19 (m, 2H), 3.91-3.95 (m, 2H), 3.96 (s, 3H), 5.61 (br, 2H), 6.49 (d, 1H), 6.66 (s, 1H), 7.14 (d, 1H), 7.34-7.37 (m, 2H), 8.38 (d, 1H), 9.10 (s, 1H).

### Example 67

### N-[2-{[4-(7-Methoxyquinazolin-4-yl)piperidin-1-yl]carbonyl}-5-(trifluoro-methoxy)phenyl]acetamide

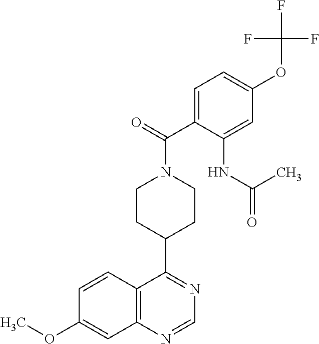 [SMILES: COc1ccc2c(C3CCN(C(=O)c4ccc(OC(F)(F)F)cc4NC(C)=O)CC3)ncnc2c1]

To a solution of (2-amino-4-(trifluoromethoxy)phenyl)(4-(7-methoxyquinazolin-4-yl)piperidin-1-yl)methanone (80 mg, 0.2 mmol), and triethylamine (60 mg, 0.6 mmol) in 2 mL of dichloromethane was added acetyl chloride (27 mg, 0.3 mmol) at 0° C. The resulting mixture was stirred at room temperature for 1 hour. Upon completion of the reaction, the solvent was removed in vacuo and the residue was purified by chromatography to give 38.6 mg (49%) of the product as a white solid. MS (ESIpos): m/z=489 (M+H)⁺; LC-MS [Method 2]: Rₜ=2.73 min. ¹H-NMR (400 MHz, DMSO-d₆): δ 1.77-1.82 (m, 1H), 1.85-1.93 (m, 3H), 2.13 (s, 3H), 2.96-3.12 (m, 1H), 3.26-3.32 (m, 1H), 3.46-3.51 (m, 1H), 3.90-3.95 (m, 1H) 3.97 (s, 3H), 4.63-4.65 (m, 1H), 7.20 (d, 1H), 7.34-7.37 (m, 2H), 7.45 (d, 1H), 7.69 (s, 1H), 8.39 (d, 1H), 9.10 (s, 1H), 9.83 (br, 1H).

### Example 68

### [4-(6-Methoxypyrido[3,4-d]pyrimidin-4-yl)piperidin-1-yl][4-(trifluoromethoxy)phenyl]methanone

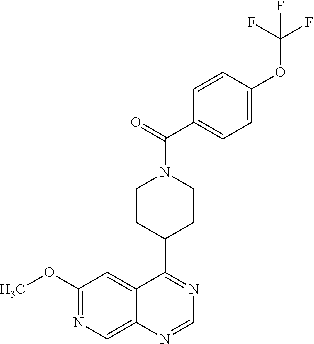 [SMILES: COc1cc2c(C3CCN(C(=O)c4ccc(OC(F)(F)F)cc4)CC3)ncnc2cn1]

### Step 1: 6-Methoxypyrido[3,4-d]pyrimidin-4(3H)-one

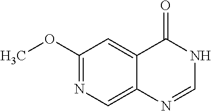

This compound was synthesized by the same method as described in example 1 to give 850 mg (80%) of the product as a brown solid. MS (ESIpos): m/z=178 (M+H)⁺. LC-MS [Method 1]: Rₜ=0.55 min.

### Step 2: 4-Chloro-6-methoxypyrido[3,4-d]pyrimidine

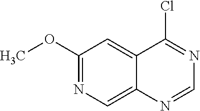

This compound was synthesized by the same method as described in example 1 to give 400 mg (40%) of the product as a dark brown solid. MS (ESIpos): m/z=196 (M+H)⁺. LC-MS [Method 1]: Rₜ=0.95 min.

### Step 3: Tert-butyl 4-(6-methoxypyrido[3,4-d]pyrimidin-4-yl)-3,6-dihydropyridine-1(2H)-carboxylate

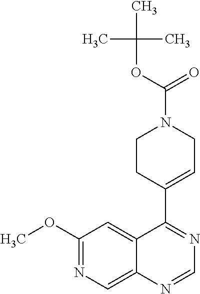 [SMILES: COc1cc2c(C3=CCN(C(=O)OC(C)(C)C)CC3)ncnc2cn1]

This compound was synthesized by the same method as described in example 1 to give 300 mg (39%) of the product as a brown solid. MS (ESIpos): m/z=343 (M+H)⁺. LC-MS [Method 2]: Rₜ=1.04 min.

### Step 4: Tert-butyl 4-(6-methoxypyrido[3,4-d]pyrimidin-4-yl)piperidine-1-carboxylate

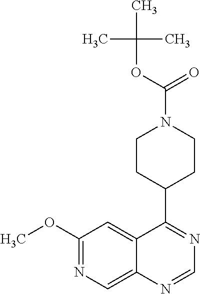 [SMILES: COc1cc2c(C3CCN(C(=O)OC(C)(C)C)CC3)ncnc2cn1]

This compound was synthesized by the same method as described in example 57 to give 40 mg (33%) of the product as a light yellow solid. MS (ESIpos): m/z=345 (M+H)⁺. LC-MS [Method 2]: Rₜ=1.17 min.

### Step 5: 6-Methoxy-4-(piperidin-4-yl)pyrido[3,4-d]pyrimidine

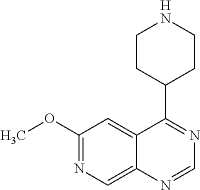

This compound was synthesized by the same method as described in example 1 (step 4) to give 40 mg (crude) of the product as a yellow solid. MS (ESIpos): m/z=245 (M+H)⁺, LC-MS [Method 2]: Rₜ=0.56 min.

### Step 6: [4-(6-Methoxypyrido[3,4-d]pyrimidin-4-yl)piperidin-1-yl][4-(trifluoro-methoxy)phenyl]methanone

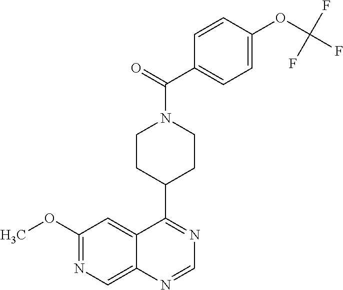 [SMILES: COc1cc2c(C3CCN(C(=O)c4ccc(OC(F)(F)F)cc4)CC3)ncnc2cn1]

This compound was synthesized by the same method as described in example 9 to give 6.6 mg of the product as an off-white solid. MS (ESIpos): m/z=433 (M+H)⁺. LC-MS [Method 3]: Rₜ=1.87 min. ¹H-NMR (400 MHz, DMSO-d₆): δ 2.00-2.05 (m, 4H), 3.20-3.21 (m, 1H), 3.47-3.48 (m, 1H), 3.85-3.88 (m, 1H), 3.96-3.99 (m, 1H), 4.09 (s, 3H), 4.77-4.81 (m, 1H), 7.40 (d, 2H), 7.53 (s, 1H), 7.60 (d, 2H), 9.12 (s, 1H), 9.16 (s, 1H).

### Example 69

### [4-(7-Methoxypyrido[2,3-d]pyrimidin-4-yl)piperidin-1-yl][4-(trifluoromethoxy)phenyl]methanone

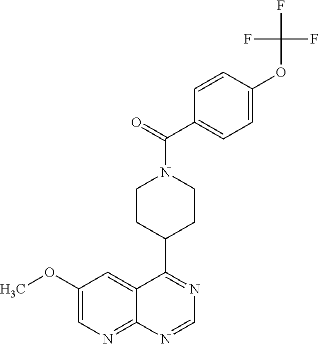 [SMILES: COc1cnc2ncnc(C3CCN(C(=O)c4ccc(OC(F)(F)F)cc4)CC3)c2c1]

### Step 1: 2-Amino-6-methoxynicotinic Acid

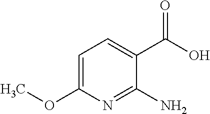

To a solution of 2-amino-6-chloronicotinic acid (900 mg, 5.4 mmol) in 25 mL methanol was added sodium methoxide (2.81 g, 52.2 mmol). The resulting mixture was stirred at reflux for two weeks. After cooled to room temperature, the solvent was removed in vacuo and the residue was purified by C18 reverse phase column [Mobile Phase A: Water, Mobile Phase B: Acetonitrile; Gradient: 0% B to 60% B in 25 min] to give 156 mg of the title compound as a white solid. MS (ESIpos): m/z=169 (M+H)⁺; LC-MS [Method 2]: Rₜ=0.66 min.

### Step 2: 7-Methoxypyrido [2,3-d] pyrimidin-4(3H)-one

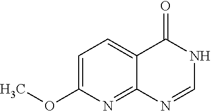

This compound was synthesized by the same method as described in example 1 to give 89 mg (49%) of the title compound as a yellow solid. MS (ESIpos): m/z=178 (M+H)⁺; LC-MS [Method 2]: Rₜ=0.74 min.

### Step 3: 4-Chloro-7-methoxypyrido [2, 3-d] pyrimidine

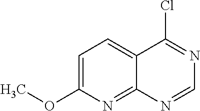

This compound was synthesized by the same method as described in example 1 to give 78 mg (63%) of the title compound as a yellow solid. MS (ESIpos): m/z=196 (M+H)⁺; LC-MS [Method 2]: Rₜ=0.81 min.

### Step 4: Tert-butyl 4-(7-methoxypyrido [2,3-d] pyrimidin-4-yl)-5, 6-dihydro-pyridine-1(2H)-carboxylate

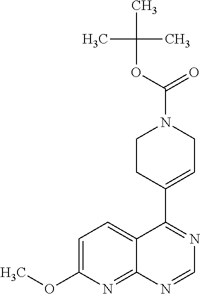 [SMILES: COc1ccc2c(C3=CCN(C(=O)OC(C)(C)C)CC3)ncnc2n1]

This compound was synthesized by the same method as described in example 1 to give 96 mg (57%) of the title compound as a yellow solid. MS (ESIpos): m/z=343 (M+H)⁺; LC-MS [Method 2]: Rₜ=1.06 min.

### Step 5: Tert-butyl 4-(7-methoxypyrido [2, 3-d] pyrimidin-4-yl) piperidine-1-carboxylate

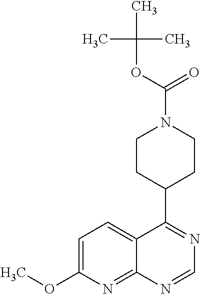 [SMILES: COc1ccc2c(C3CCN(C(=O)OC(C)(C)C)CC3)ncnc2n1]

This compound was synthesized by the same method as described in example 1 to give 67 mg (26%) of the title compound as a yellow solid. MS (ESIpos): m/z=345 (M+H)⁺; LC-MS [Method 2]: Rₜ=1.17 min.

### Step 6: 7-Methoxy-4-(piperidin-4-yl) pyrido [2, 3-d] pyrimidine

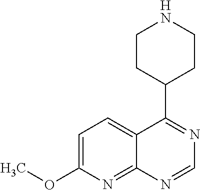

This compound was synthesized by the same method as described in example 1 to give 24.0 mg (66%) of the title compound as a yellow solid. MS (ESIpos): m/z=245 (M+H)⁺; LC-MS [Method 2]: Rₜ=0.31 min.

### Step 7: [4-(7-Methoxypyrido[2,3-d]pyrimidin-4-yl)piperidin-1-yl][4-(trifluoro-methoxy)phenyl]methanone

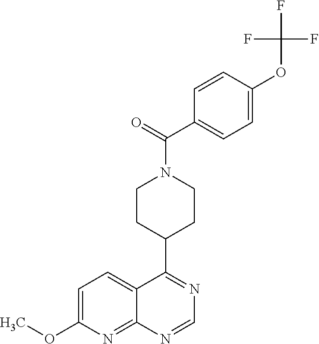 [SMILES: COc1ccc2c(C3CCN(C(=O)c4ccc(OC(F)(F)F)cc4)CC3)ncnc2n1]

This compound was synthesized by the same method as described in example 9 to give 6.2 mg (14%) of the title compound as a white solid. MS (ESIpos): m/z=433 (M+H)⁺; LC-MS [Method 2]: Rₜ=1.50 min. ¹H-NMR (400 MHz, DMSO-d₆): δ 1.82-1.90 (m, 4H), 3.01-3.09 (m, 1H), 3.32-3.38 (m, 1H), 3.61-3.66 (m, 1H), 3.92-3.98 (m, 1H), 3.99 (s, 3H), 3.52-3.61 (m, 1H), 7.25 (d, 1H), 7.45 (d, 2H), 7.59 (d, 2H), 7.79 (d, 1H), 9.23 (s, 1H).

### Example 70

### [4-(7-Methoxypyrido[3,2-d]pyrimidin-4-yl)piperidin-1-yl][4-(trifluoromethoxy)phenyl]methanone

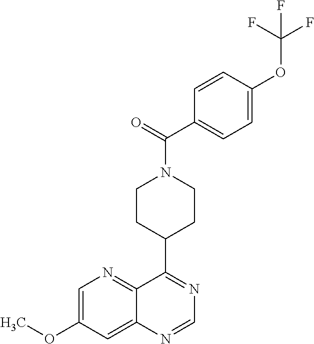 [SMILES: COc1cnc2c(C3CCN(C(=O)c4ccc(OC(F)(F)F)cc4)CC3)ncnc2c1]

### Step 1: Tert-butyl N-(5-bromo-2-chloropyridin-3-yl)-N-[(tert-butoxy)carbonyl]-carbamate

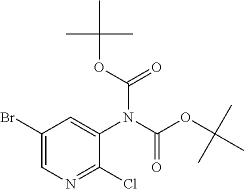 [SMILES: CC(C)(C)OC(=O)N(C(=O)OC(C)(C)C)c1cc(Br)cnc1Cl]

To a solution of 5-bromo-2-chloropyridin-3-amine (1000 g, 4.82 mol) and trimethylamine (1961 g, 19, 38 mmol) in 10 L dichloromethane were added under nitrogen atmosphere 4-dimethylaminopyridine (59 g, 482.93 mmol). This was followed by the addition of (Boc)₂O (3703 g, 16.97 mol) in several batches. The resulting solution was stirred overnight at 25° C. The resulting mixture was concentrated under vacuum. The residue was applied onto a silica gel column with dichloromethane/petroleum ether (4:1). This resulted in 1.3 kg (66%) of tert-butyl N-(5-bromo-2-chloropyridin-3-yl)-N-[(tert-butoxy)carbonyl]carbamate as a white solid.

### Step 2: 5-[bis[(Tert-butoxy)carbonyl]amino]-6-chloropyridin-3-yl)boronic Acid

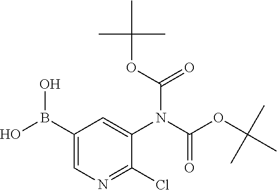 [SMILES: CC(C)(C)OC(=O)N(C(=O)OC(C)(C)C)c1cc(B(O)O)cnc1Cl]

To a mixture of tert-butyl N-(5-bromo-2-chloropyridin-3-yl)-N-[(tert-butoxy)carbonyl]carbamate (500 g, 1.23 mol) and (MeO)₃B (384 g, 3.69 mol) in tetrahydrofuran (10 L) under inert atmosphere was added dropwise a solution of n-BuLi (2.5M) (1477 mL) with stirring at −85-−80° C. The resulting solution was stirred for 2 h at −85-−80° C. The reaction was then quenched by the addition of 3000 mL of saturated aqueous NH₄Cl. The pH value of the solution was adjusted to 3-4 with hydrogen chloride (2 mol/L). The resulting solution was extracted with 4×3 L of ethyl acetate and the organic layers combined and dried over anhydrous sodium sulfate and concentrated under vacuum. This resulted in 300 g (66%) of (5-[bis[(tert-butoxy)carbonyl]amino]-6-chloropyridin-3-yl)boronic acid as a white solid.

### Step 3: Tert-butyl N-[(tert-butoxy)carbonyl]-N-(2-chloro-5-hydroxypyridin-3-yl)carbamate

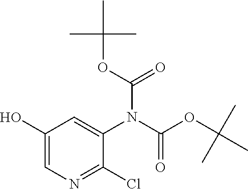 [SMILES: CC(C)(C)OC(=O)N(C(=O)OC(C)(C)C)c1cc(O)cnc1Cl]

To a solution of (5-[bis[(tert-butoxy)carbonyl]amino]-6-chloropyridin-3-yl)boronic acid (600 g, 1.61 mol) in tetrahydrofuran (2000 mL) was added sodium hydroxide (2 N) (600 mL), followed by H₂O₂ (30%) (3000 mL) dropwise with stirring at 0° C. The resulting solution was stirred for 3 h at 25° C. The pH value of the solution was adjusted to 3-4 with hydrogen chloride (2 mol/L). The resulting solution was extracted with 4×3 L of ethyl acetate and the organic layers combined and dried over anhydrous sodium sulfate and concentrated under vacuum. This resulted in 450 g (81%) of tert-butyl N-[(tert-butoxy)carbonyl]-N-(2-chloro-5-hydroxypyridin-3-yl)carbamate as a white solid.

### Step 4: Tert-butyl N-[(tert-butoxy)carbonyl]-N-(2-chloro-5-methoxypyridin-3-yl)carbamate

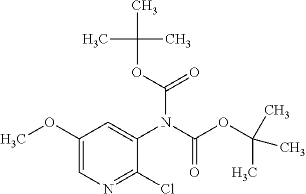 [SMILES: COc1cnc(Cl)c(N(C(=O)OC(C)(C)C)C(=O)OC(C)(C)C)c1]

To a solution of tert-butyl N-[(tert-butoxy)carbonyl]-N-(2-chloro-5-hydroxypyridin-3-yl)carbamate (450 g, 1.31 mol) in N,N-dimethylformamide (5000 mL) was added under inert atmosphere sodium carbonate (693 g, 6.54 mol, followed by CH₃I (929 g, 6.54 mol) dropwise with stirring at 25° C. The resulting solution was stirred for 12 h at 25° C. The resulting solution was diluted with 4000 mL of H₂O. The resulting solution was extracted with 5×3 L of ethyl acetate and the organic layers combined. The resulting mixture was washed with 6×2000 mL of brine. The mixture was dried over anhydrous sodium sulfate and concentrated under vacuum. The residue was applied onto a silica gel column with ethyl acetate/petroleum ether (1:1). This resulted in 350 g (75%) of tert-butyl N-[(tert-butoxy)carbonyl]-N-(2-chloro-5-methoxypyridin-3-yl)carbamate as a light yellow solid.

### Step 5: 3-[[(Tert-butoxy)carbonyl]amino]-5-methoxypyridine-2-carboxylate

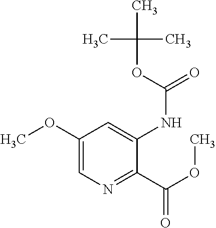

Under inert atmosphere CO was added to a mixture of tert-butyl N-[(tert-butoxy)carbonyl]-N-(2-chloro-5-methoxypyridin-3-yl)carbamate (350 g, 975 mmol), TEA (197.5 g, 1.95 mol) and Pd(dppf)Cl₂ (7.2 g, 9.84 mmol) in methanol (3500 mL). The resulting solution was stirred for 12 h at 90° C. The resulting mixture was concentrated under vacuum. The residue was applied onto a silica gel column with ethyl acetate/hexane (1:1). This resulted in 250 g (91%) of methyl 3-[[(tert-butoxy)carbonyl]amino]-5-methoxypyridine-2-carboxylate as a white solid.

### Step 6. 3-[[(Tert-Butoxy)carbonyl]amino]-5-methoxypyridine-2-carboxylic Acid

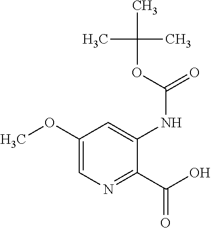 [SMILES: COc1cnc(C(=O)O)c(NC(=O)OC(C)(C)C)c1]

To a solution of methyl 3-[[(tert-butoxy)carbonyl]amino]-5-methoxypyridine-2-carboxylate (250 g, 885.6 mmol) in tetrahydrofuran (2000 mL) was added dropwise a solution of sodium hydroxide (67.6 g, 1.69 mol) in water (500 mL) at 0° C. The resulting solution was stirred for 2 h at 25° C. The resulting mixture was concentrated under vacuum. The resulting solution was diluted with 500 mL of H₂O. The pH value of the solution was adjusted to 3-4 with hydrogen chloride (2 mol/L). The solids were collected by filtration. The aqueous layer was extracted again with THF/EA (1:1). The combined organic layer was dried over anhydrous sodium sulfate and concentrated under vacuum. This resulted in 200 g (84%) of 3-[[(tert-butoxy)carbonyl]amino]-5-methoxypyridine-2-carboxylic acid as a white solid.

### Step 7: 3-Amino-5-methoxypyridine-2-carboxylic Acid; Trifluoroacetic Acid

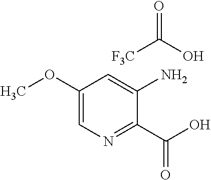

To a solution of 3-[[(tert-butoxy)carbonyl]amino]-5-methoxypyridine-2-carboxylic acid (200 g, 745.53 mmol) in dichloromethane (1000 mL) was added trifluoroacetic acid (800 mL). The resulting solution was stirred for 2 h at 25° C. The resulting mixture was concentrated under vacuum. The resulting solution was diluted with 2 L of ether. The solids were collected by filtration. This resulted in 190 g (90%) of 3-amino-5-methoxypyridine-2-carboxylic acid; trifluoroacetic acid as a brown solid.

### Step 8: 7-Methoxy-3H,4H-pyrido[3,2-d]pyrimidin-4-one

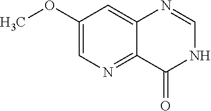

To a mixture of 3-amino-5-methoxypyridine-2-carboxylic acid; trifluoroacetic acid (190 g, 673.34 mmol), in 2-methoxyethanol (2500 mL) was added formamidine acetate (245 g, 3.50 equiv). The resulting solution was stirred for 5 h at 120° C. The resulting mixture was concentrated under vacuum. The resulting solution was diluted with 1 L of H₂O. This resulted in 80 g (67%) of 7-methoxy-3H,4H-pyrido[3,2-d]pyrimidin-4-one as a brown solid.

### Step 9: 4-Chloro-7-methoxypyrido[3,2-d]pyrimidine

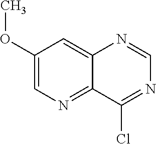

To a solution of 7-methoxy-3H,4H-pyrido[3,2-d]pyrimidin-4-one (80 g, 451 mmol), in toluene (800 mL) were added DIEA (160 mL) and POCl₃ (160 mL). The resulting solution was stirred for 2 h at 90° C. The resulting mixture was concentrated under vacuum. The residue was applied onto a silica gel column with ethyl acetate/petroleum ether (1:1). This resulted in 80 g (91%) of 4-chloro-7-methoxypyrido[3,2-d]pyrimidine as a white solid.

### Step 10: Tert-butyl 4-[7-methoxypyrido[3,2-d]pyrimidin-4-yl]-1,2,3,6-tetrahydropyridine-1-carboxylate

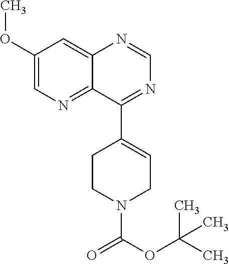 [SMILES: COc1cnc2c(C3=CCN(C(=O)OC(C)(C)C)CC3)ncnc2c1]

To a mixture of 4-chloro-7-methoxypyrido[3,2-d]pyrimidine (70 g, 358 mmol), tert-butyl 4-(tetramethyl-1,3,2-dioxaborolan-2-yl)-1,2,3,6-tetrahydropyridine-1-carboxylate (222 g, 717 mmol) in dioxane (2000 mL) were added under inert atmosphere a solution of sodium carbonate (76 g, 718 mmol) in water (500 mL), followed by the Pd(PPh₃)₄ (5 g, 4.33 mmol). The flask was evacuated and flushed three times with nitrogen. The resulting solution was stirred for 5 h at 100° C. The reaction mixture was cooled. The resulting solution was extracted with 3×1 L of ethyl acetate and the organic layers combined. The resulting mixture was washed with 3×500 mL of brine. The mixture was dried over anhydrous sodium sulfate and concentrated under vacuum. The residue was applied onto a silica gel column with ethyl acetate/petroleum ether (1:1). This resulted in 96 g (78%) of tert-butyl 4-[7-methoxypyrido[3,2-d]pyrimidin-4-yl]-1,2,3,6-tetrahydropyridine-1-carboxylate as a light yellow solid.

H-NMR (300 MHz, DMSO-d₆) δ 9.193 (s, 1H), 8.811 (d, J=1.35 Hz, 1H), 7.740 (d, J=1.35 Hz, 1H), 7.302 (s, 1H), 4.177 (s, 2H), 4.028 (s, 3H), 3.592 (m, 2H), 2.781 (s, 2H), 1.449 (s, 9H).

### Step 11: Tert-butyl 4-[7-methoxypyrido[3,2-d]pyrimidin-4-yl]piperidine-1-carboxylate

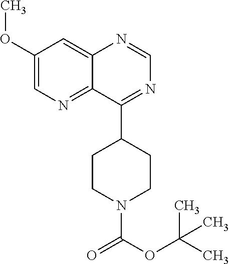 [SMILES: COc1cnc2c(C3CCN(C(=O)OC(C)(C)C)CC3)ncnc2c1]

To a solution of tert-butyl 4-[7-methoxypyrido[3,2-d]pyrimidin-4-yl]-1,2,3,6-tetrahydropyridine-1-carboxylate (50 g, 146.03 mmol) in methanol (500 mL) was added palladium on carbon (1 g). The flask was evacuated and flushed three times with nitrogen, followed by flushing with hydrogen. The resulting solution was stirred for 2 h at 25° C. After filtration the mixture was concentrated under vacuum. The residue was purified by chromatography with ethyl acetate/petroleum ether (1:2). This resulted in 45 g (89%) of tert-butyl 4-[7-methoxypyrido[3,2-d]pyrimidin-4-yl]piperidine-1-carboxylate as a off-white solid.

H-NMR (300 MHz, DMSO-d₆): δ 9.192 (s, 1H), 8.837 (d, J=1.35 Hz, 1H), 7.746 (d, J=1.5 Hz, 1H), 4.246 (m, 1H), 4.113 (m, 2H), 4.028 (s, 3H), 2.958 (s, 2H), 1.792 (m, 4H), 1.430 (s, 9H).

### Step 12: 4-[7-Methoxypyrido[3,2-d]pyrimidin-4-yl]piperidine dihydrochloride

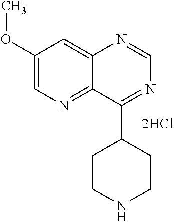 [SMILES: COc1cnc2c(C3CCNCC3)ncnc2c1]

To a solution of tert-butyl 4-[7-methoxypyrido[3,2-d]pyrimidin-4-yl]piperidine-1-carboxylate (35 g, 101.62 mmol) in dichloromethane (300 mL) was added hydrogen chloride (in dioxane, 4M) (150 mL). The resulting solution was stirred for 2 h at 25° C. The resulting mixture was concentrated under vacuum. The crude product was purified by re-crystallization from ether. This resulted in 30.0 g (93%) of 4-[7-methoxypyrido[3,2-d]pyrimidin-4-yl]piperidine dihydrochloride as a light brown solid.

H-NMR: (300 MHz, CDC₃, ppm): δ9.230 (s, 2H), 9.042 (s, 1H), 8.850 (s, 1H), 8.841 (s, 1H), 7.770 (d, J=3.0 Hz, 1H), 4.353 (m, 1H), 4.035 (s, 3H), 3.399 (d, J=6.0 Hz, 2H), 3.159 (m, 2H), 2.118 (m, 4H). LC-MS-: (ES, m/z): 245[M−2HCl+H]⁺

### Step 13: [4-(7-Methoxypyrido[3,2-d]pyrimidin-4-yl)piperidin-1-yl][4-(trifluoro-methoxy)phenyl]-methanone

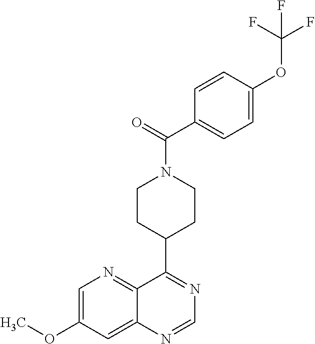 [SMILES: COc1cnc2c(C3CCN(C(=O)c4ccc(OC(F)(F)F)cc4)CC3)ncnc2c1]

This compound was synthesized by the same method as described in example 9 to give 10.5 mg (8%) of the title compound as a white solid. MS (ESIpos): m/z=433 (M+H)⁺; LC-MS [Method 2]: Rₜ=2.20 min. ¹H-NMR (400 MHz, DMSO-d₆): δ 1.84-1.96 (m, 4H), 3.01-3.06 (m, 1H), 3.32-3.39 (m, 1H), 3.61-3.70 (m, 1H), 4.03 (s, 3H), 4.33-4.37 (m, 1H), 4.62-4.64 (m, 1H), 7.44 (d, 2H), 7.55 (d, 2H), 7.84 (s, 1H), 8.83 (s, 1H), 9.20 (s, 1H).

### Example 71

### (4-{7-[(1-Methylpiperidin-4-yl)amino]pyrido[3,2-d]pyrimidin-4-yl}piperidin-1-yl)[4-(trifluoromethoxy)phenyl]methanone

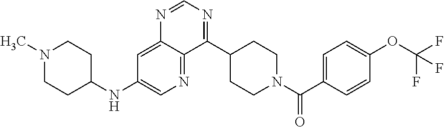 [SMILES: CN1CCC(Nc2cnc3c(C4CCN(C(=O)c5ccc(OC(F)(F)F)cc5)CC4)ncnc3c2)CC1]

### Step 1: 2,2,2-Trifluoro-1-(4-[7-methoxypyrido[3,2-d]pyrimidin-4-yl]piperidin-1-yl)ethan-1-one

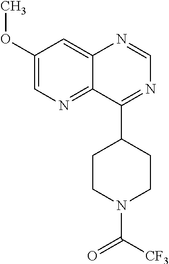 [SMILES: COc1cnc2c(C3CCN(C(=O)C(F)(F)F)CC3)ncnc2c1]

To a solution of 4-[7-methoxypyrido[3,2-d]pyrimidin-4-yl]piperidine dihydrochloride (35 g, 110 mmol) and TEA (56 g, 553 mmol) in dichloromethane (350 mL) was added dropwise at 0° C. TFAA (46.62 g, 221.97 mmol,). The resulting solution was stirred for 1 h at 25° C. The resulting mixture was concentrated under vacuum. The residue was purified by chromatography to give 37 g (99%) of 2,2,2-trifluoro-1-(4-[7-methoxypyrido[3,2-d]pyrimidin-4-yl]piperidin-1-yl)ethan-1-one as a white solid.

### Step 2: 2,2,2-Trifluoro-1-(4-[7-hydroxypyrido[3,2-d]pyrimidin-4-yl]piperidin-1-yl)ethan-1-one

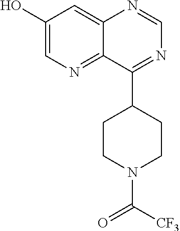 [SMILES: O=C(N1CCC(c2ncnc3cc(O)cnc23)CC1)C(F)(F)F]

To a solution of 2,2,2-trifluoro-1-(4-[7-methoxypyrido[3,2-d]pyrimidin-4-yl]piperidin-1-yl)ethan-1-one (37 g, 109 mmol) in 1,2-dichloroethane (300 mL) was added under inert atmosphere boron tribromide (70 mL). The resulting solution was stirred for 6 h at 90° C. The resulting mixture was concentrated under vacuum. The reaction was then quenched by the addition of 200 mL of MeOH. which was used in the next step without further purification.

### Step 3: 4-(Piperidin-4-yl)pyrido[3,2-d]pyrimidin-7-ol

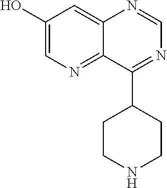 [SMILES: Oc1cnc2c(C3CCNCC3)ncnc2c1]

To a solution of 2,2,2-Trifluoro-1-(4-[7-hydroxypyrido[3,2-d]pyrimidin-4-yl]piperidin-1-yl)ethan-1-one was added sodium hydroxide (2 N) (250 mL, 6.25 mol). The resulting solution was stirred for 1 h at 25° C. which was used in the next step without further purification.

### Step 4: tert-Butyl 4-[7-hydroxypyrido[3,2-d]pyrimidin-4-yl]piperidine-1-carboxylate

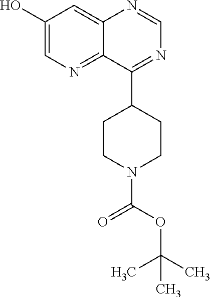 [SMILES: CC(C)(C)OC(=O)N1CCC(c2ncnc3cc(O)cnc23)CC1]

To a solution of 4-(Piperidin-4-yl)pyrido[3,2-d]pyrimidin-7-ol was added (Boc)₂O (47.5 g, 218 mmol). The resulting solution was stirred for 1 h at 25° C. The pH value of the solution was adjusted to 5-6 with hydrogen chloride (1 mol/L). The resulting solution was extracted with 2×500 mL of ethyl acetate and the organic layers combined and dried over anhydrous sodium sulfate and concentrated under vacuum. The residue was purified by chromatography to give 30 g of tert-butyl 4-[7-hydroxypyrido[3,2-d]pyrimidin-4-yl]piperidine-1-carboxylate as a light yellow solid.

### Step 5: 4-(Piperidin-4-yl)pyrido[3,2-d]pyrimidin-7-ol dihydrochloride

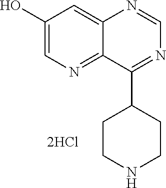 [SMILES: Oc1cnc2c(C3CCNCC3)ncnc2c1]

To a solution of tert-butyl 4-[7-hydroxypyrido[3,2-d]pyrimidin-4-yl]piperidine-1-carboxylate (29 g, 88 mmol) in dichloromethane (200 mL) was added a solution of hydrogen chloride (4M in dioxane, 50 mL). The resulting solution was stirred for 1 h at 25° C. The resulting mixture was concentrated under vacuum. The resulting solution was diluted with 200 mL of ether. This resulted in 25 g (94%) of 4-(piperidin-4-yl)pyrido[3,2-d]pyrimidin-7-ol dihydrochloride as a light yellow solid.

### Step 6: 4-(1-[[4-(Trifluoromethoxy)phenyl]carbonyl]piperidin-4-yl)pyrido[3,2-d]pyrimidin-7-ol

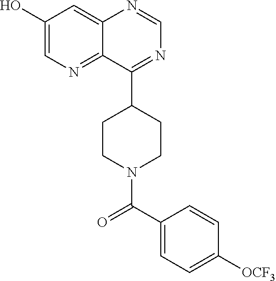 [SMILES: O=C(c1ccc(OC(F)(F)F)cc1)N1CCC(c2ncnc3cc(O)cnc23)CC1]

To a solution 4-(trifluoromethoxy)benzoyl chloride (36.6 g, 125.00 mmol, 1.00 equiv) in dichloromethane (500 mL) was added under inert atmosphere at −10° C. TEA (127 g, 1.26 mol). After 10 minutes 4-(piperidin-4-yl)pyrido[3,2-d]pyrimidin-7-ol dihydrochloride (37 g, 163 mmol) was added dropwise at −10° C. The resulting solution was stirred for 1 h at 25° C. The resulting mixture was concentrated under vacuum. The resulting solution was diluted with 400 mL of H₂O. The pH value of the solution was adjusted to 5-6 with hydrogen chloride (2 mol/L). The resulting solution was extracted with 3×500 mL of ethyl acetate and the organic layers combined. The resulting mixture was washed with 4×500 mL of brine. The mixture was dried over anhydrous sodium sulfate and concentrated under vacuum. The residue purified by chromatography to give 25 g (35%) of the desired product as a off-white solid.

¹H-NMR (300 MHz, CDC₃, ppm) δ11.491 (s, 1H), 9.123 (s, 1H), 8.760 (d, J=1.35 Hz, 1H), 7.584 (d, J=4.35 Hz, 2H), 7.455 (m, 3H), 4.420 (m, 2H), 3.683 (s, 1H), 3.040 (s, 1H), 1.865 (m, 4H). LC-MS (ES, m/z): 419[M+H]⁺

### Step 7: 4-{1-[4-(trifluoromethoxy)benzoyl]piperidin-4-yl}pyrido[3,2-d]pyrimidin-7-yl 1,1,2,2,3,3,4,4,4-nonafluorobutane-1-sulfonate

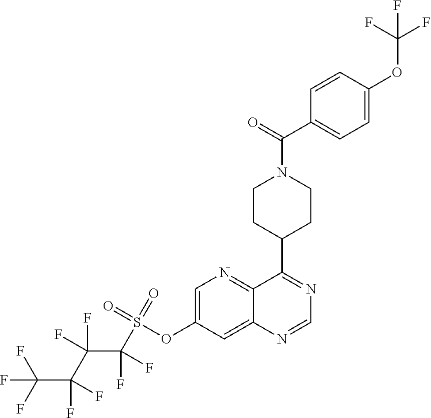 [SMILES: O=C(c1ccc(OC(F)(F)F)cc1)N1CCC(c2ncnc3cc(OS(=O)(=O)C(F)(F)C(F)(F)C(F)(F)C(F)(F)F)cnc23)CC1]

To a solution of [4-(7-hydroxypyrido[3,2-d]pyrimidin-4-yl)piperidin-1-yl][4-(trifluoromethoxy)phenyl]methanone (2.0 g, 4.78 mmol) in tetrahydrofuran (50 mL) was added 1,1,2,2,3,3,4,4,4-nonafluorobutane-1-sulfonyl fluoride (1.26 mL, 7.17 mmol) and potassium carbonate (1.98 g, 14.3 mmol). The reaction mixture was refluxed for one hour. Saturated aqueous sodium chloride solution was added at room temperature. The aqueous phase was extracted three times with ethyl acetate. The combined organic phases were dried over sodium sulfate. Subsequent filtration and removal of the solvent, the crude product was used in the next step without prior purification.

### Step 8: (4-{7-[(1-methylpiperidin-4-yl)amino]pyrido[3,2-d]pyrimidin-4-yl}piperidin-1-yl)[4-(trifluoromethoxy)phenyl]methanone

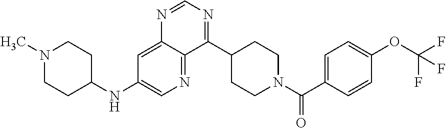 [SMILES: CN1CCC(Nc2cnc3c(C4CCN(C(=O)c5ccc(OC(F)(F)F)cc5)CC4)ncnc3c2)CC1]

To a solution of 4-{1-[4-(trifluoromethoxy)benzoyl]piperidin-4-yl}pyrido[3,2-d]pyrimidin-7-yl 1,1,2,2,3,3,4,4,4-nonafluorobutane-1-sulfonate (100 mg, 0.14 mmol) in toluene (1.5 mL) in a sealed tube were added 1-methylpiperidin-4-amine (36 μL, 0.29 mmol), sodium tert-butoxide (55 mg, 0.57 mmol), tris(dibenzylideneacetone)-dipalladium-chloroform adduct (7.4 mg, 0.007 mmol) and 4,5-bis(diphenylphosphino)-9,9-dimethylxanthene (8.3 mg, 0.014 mmol). The reaction mixture was allowed to stir 3 h at 120° C. After cooling to room temperature, the mixture was filtered over Celite, washed with toluene followed by the removal of the solvent. The crude product was purified by chromatography to afford the desired product (8 mg, 10%).

¹H NMR (400 MHz, DMSO-d₆) δ 8.92 (s, 1H), 8.58 (d, J=2.79 Hz, 1H), 7.53-7.62 (m, 2H), 7.41-7.48 (m, 2H), 7.22 (d, J=7.60 Hz, 1H), 6.93 (d, J=2.53 Hz, 1H), 4.52-4.67 (m, 1H), 4.15-4.31 (m, 1H), 3.61-3.74 (m, 1H), 3.38-3.49 (m, 1H), 2.94-3.07 (m, 1H), 2.70-2.81 (m, 2H), 2.18 (s, 3H), 2.02-2.14 (m, 2H), 1.69-1.99 (m, 7H), 1.40-1.58 (m, 2H). LC-MS [Method 4]: Rₜ=0.73 min; MS (ESIpos): m/z=501.2 [M+H]⁺.

### Example 72

### {4-[7-(3,3-Difluoropyrrolidin-1-yl)pyrido[3,2-d]pyrimidin-4-yl]piperidin-1-yl}[4-(trifluoromethoxy)phenyl]methanone

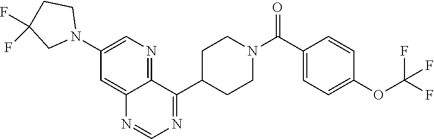 [SMILES: O=C(c1ccc(OC(F)(F)F)cc1)N1CCC(c2ncnc3cc(N4CCC(F)(F)C4)cnc23)CC1]

This compound was synthesized by the same method as described in example 71 to afford the desired product in 5% yields (4 mg)

¹H NMR (400 MHz, DMSO-d₆) δ 9.03 (s, 1H), 8.74 (d, J=3.04 Hz, 1H), 7.56-7.61 (m, 2H), 7.43-7.48 (m, 2H), 7.14 (d, J=2.79 Hz, 1H), 4.53-4.71 (m, 1H), 4.27-4.39 (m, 1H), 4.03 (t, J=12.93 Hz, 2H), 3.78 (t, J=7.35 Hz, 2H), 3.64-3.74 (m, 1H), 2.91-3.11 (m, 1H), 2.57-2.72 (m, 2H), 1.75-2.07 (m, 5H). One proton was not visible due to overlap with water. LC-MS [Method 4]: Rₜ=1.2 min; MS (ESIpos): m/z=508.2 [M+H]⁺.

### Example 73

### {4-[7-(4-Amino-4-methylpiperidin-1-yl)pyrido[3,2-d]pyrimidin-4-yl]piperidin-1-yl}[4-(trifluoromethoxy)phenyl]methanone

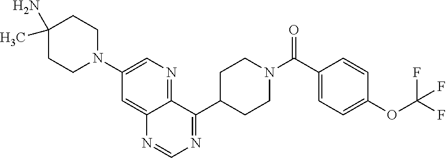 [SMILES: CC1(N)CCN(c2cnc3c(C4CCN(C(=O)c5ccc(OC(F)(F)F)cc5)CC4)ncnc3c2)CC1]

This compound was synthesized by the same method as described in example 71 to afford the desired product in 7% yields (6 mg).

¹H NMR (400 MHz, DMSO-d₆) δ9.04 (d, J=3.04 Hz, 1H), 9.00 (s, 1H), 7.56-7.61 (m, 2H), 7.42-7.48 (m, 2H), 7.34 (d, J=3.04 Hz, 1H), 4.55-4.70 (m, 1H), 4.22-4.35 (m, 1H), 3.48-3.75 (m, 5H), 3.18-3.29 (m, 1H), 2.93-3.09 (m, 1H), 1.43-2.05 (m, 10H).

### Example 74

### {4-[7-(Morpholin-4-yl)pyrido[3,2-d]pyrimidin-4-yl]piperidin-1-yl}[4-(trifluoromethoxy)phenyl]methanone

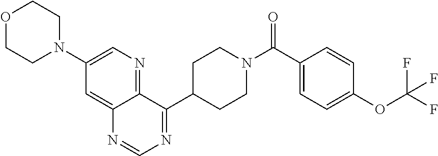 [SMILES: O=C(c1ccc(OC(F)(F)F)cc1)N1CCC(c2ncnc3cc(N4CCOCC4)cnc23)CC1]

This compound was synthesized by the same method as described in example 71 to afford the desired product in 25% yields (41 mg).

¹H NMR (500 MHz, DMSO-d₆) δ 9.04 (s, 1H), 9.01 (d, J=2.86 Hz, 1H), 7.56-7.59 (m, 2H), 7.40-7.44 (m, 2H), 7.38 (d, J=2.86 Hz, 1H), 4.29-4.38 (m, 1H), 4.07-4.27 (m, 1H), 3.75-3.84 (m, 4H), 3.46-3.54 (m, 4H), 3.15-3.26 (m, 2H), 1.87-1.96 (m, 4H). One proton was not visible. LC-MS [Method 4]: Rₜ=1.08 min; MS (ESIpos): m/z=488.2 [M+H]⁺.

### Example 75

### (4-{7-[(3-Hydroxy-3-methylpyrrolidin-1-yl]pyrido[3,2-d]pyrimidin-4-yl}piperidin-1-yl)[4-(trifluoromethoxy)phenyl]methanone

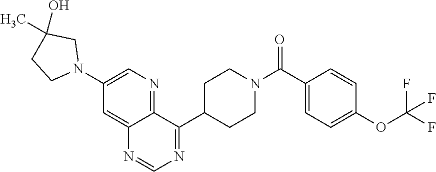 [SMILES: CC1(O)CCN(c2cnc3c(C4CCN(C(=O)c5ccc(OC(F)(F)F)cc5)CC4)ncnc3c2)C1]

This compound was synthesized by the same method as described in example 71 to afford the desired product in 7% yields (9 mg).

¹H NMR (400 MHz, DMSO-d₆) δ8.95 (s, 1H), 8.67 (d, J=2.79 Hz, 1H), 7.54-7.63 (m, 2H), 7.41-7.49 (m, 2H), 6.91 (d, J=2.79 Hz, 1H), 4.96 (s, 1H), 4.58-4.71 (m, 1H), 4.25-4.39 (m, 1H), 3.54-3.77 (m, 3H), 3.37-3.47 (m, 2H), 2.93-3.11 (m, 1H), 1.77-2.04 (m, 6H), 1.40 (s, 3H). 1 aliphatic proton was not visible. LC-MS [Method 4]: Rₜ=1.0 min; MS (ESIpos): m/z=502.1 [M+H]⁺.

### Example 76

### (4-{7-[3-(Dimethylamino)pyrrolidin-1-yl]pyrido[3,2-d]pyrimidin-4-yl}piperidin-1-yl)[4-(trifluoromethoxy)phenyl]methanone

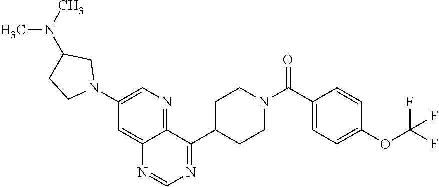 [SMILES: CN(C)C1CCN(c2cnc3c(C4CCN(C(=O)c5ccc(OC(F)(F)F)cc5)CC4)ncnc3c2)C1]

This compound was synthesized by the same method as described in example 71 to afford the desired product in 9% yields (12 mg).

¹H NMR (400 MHz, DMSO-d₆) δ8.97 (s, 1H), 8.71 (d, J=2.79 Hz, 1H), 7.56-7.60 (m, 2H), 7.43-7.48 (m, 2H), 6.99 (d, J=2.79 Hz, 1H), 4.57-4.70 (m, 1H), 4.20-4.36 (m, 1H), 3.63-3.80 (m, 3H), 3.42-3.52 (m, 1H), 3.19-3.30 (m, 2H), 2.94-3.10 (m, 1H), 2.81-2.91 (m, 1H), 2.23 (m, 6H), 1.71-1.98 (m, 6H). LC-MS [Method 4]: Rₜ=0.72 min; MS (ESIpos): m/z=515.2 [M+H]⁺.

### Example 77

### {4-[7-(4,4-Difluoropiperidin-1-yl)pyrido[3,2-d]pyrimidin-4-yl]piperidin-1-yl}[4-(trifluoromethoxy)phenyl]methanone

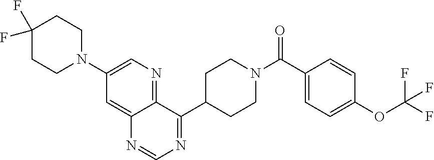 [SMILES: O=C(c1ccc(OC(F)(F)F)cc1)N1CCC(c2ncnc3cc(N4CCC(F)(F)CC4)cnc23)CC1]

This compound was synthesized by the same method as described in example 71 to afford the desired product in 8% yields (13 mg).

¹H NMR (400 MHz, DMSO-d₆) δ9.10 (d, J=3.04 Hz, 1H), 9.06 (s, 1H), 7.56-7.60 (m, 2H), 7.52 (d, J=2.79 Hz, 1H), 7.44-7.48 (m, 2H), 4.57-4.71 (m, 1H), 4.27-4.38 (m, 1H), 3.59-3.80 (m, 5H), 2.96-3.10 (m, 1H), 1.75-2.22 (m, 8H). One aliphatic proton was not visible. LC-MS [Method 4]: Rₜ=1.23 min; MS (ESIpos): m/z=522.1 [M+H]⁺.

### Example 78

### {4-[7-(2,2-Dimethylmorpholin-4-yl)pyrido[3,2-d]pyrimidin-4-yl]piperidin-1-yl}[4-(trifluoromethoxy)phenyl]methanone

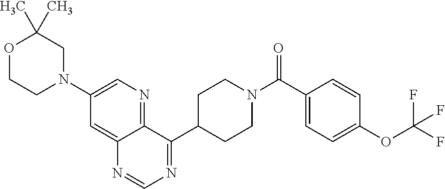 [SMILES: CC1(C)CN(c2cnc3c(C4CCN(C(=O)c5ccc(OC(F)(F)F)cc5)CC4)ncnc3c2)CCO1]

This compound was synthesized by the same method as described in example 71 to afford the desired product in 4% yields (6 mg).

¹H NMR (400 MHz, DMSO-d₆) δ9.02-9.05 (m, 2H), 7.56-7.60 (m, 2H), 7.44-7.48 (m, 2H), 7.38 (d, J=3.04 Hz, 1H), 4.54-4.70 (m, 1H), 4.21-4.36 (m, 1H), 3.75-3.83 (m, 2H), 3.63-3.72 (m, 1H), 3.48-3.55 (m, 2H), 3.37-3.44 (m, 3H), 2.98-3.10 (m, 1H), 1.76-2.06 (m, 4H), 1.22 (s, 6H). LC-MS [Method 4]: Rₜ=1.19 min; MS (ESIpos): m/z=516.2 [M+H]⁺.

### Example 79

### {4-[7-(4-Methoxypiperidin-1-yl)pyrido[3,2-d]pyrimidin-4-yl]piperidin-1-yl}[4-(trifluoromethoxy)phenyl]methanone

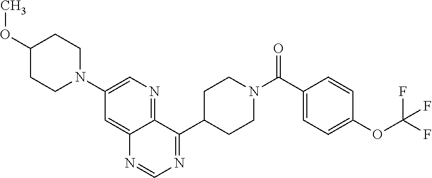 [SMILES: COC1CCN(c2cnc3c(C4CCN(C(=O)c5ccc(OC(F)(F)F)cc5)CC4)ncnc3c2)CC1]

This compound was synthesized by the same method as described in example 71 to afford the desired product in 10% yields (14 mg).

¹H NMR (400 MHz, DMSO-d₆) δ9.05 (d, J=2.79 Hz, 1H), 9.02 (s, 1H), 7.56-7.60 (m, 2H), 7.44-7.48 (m, 2H), 7.38 (d, J=3.04 Hz, 1H), 4.62-4.73 (m, 1H), 4.24-4.37 (m, 1H), 3.77-3.90 (m, 2H), 3.65-3.74 (m, 1H), 3.44-3.52 (m, 1H), 3.26-3.32 (m, 6H), 2.97-3.12 (m, 1H), 1.76-2.02 (m, 6H), 1.50-1.61 (m, 2H). LC-MS [Method 4]: Rₜ=1.17 min; MS (ESIpos): m/z=516.2 [M+H]⁺.

### Example 80

### {4-[7-(4-Methylpiperazin-1-yl)pyrido[3,2-d]pyrimidin-4-yl]piperidin-1-yl}[4-(trifluoromethoxy)-phenyl]methanone

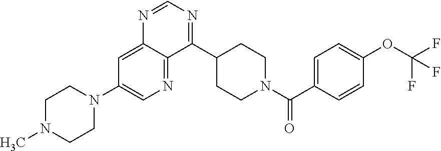 [SMILES: CN1CCN(c2cnc3c(C4CCN(C(=O)c5ccc(OC(F)(F)F)cc5)CC4)ncnc3c2)CC1]

This compound was synthesized by the same method as described in example 71 to afford the desired product in 33% yields (24 mg).

¹H NMR (400 MHz, DMSO-d₆) δ9.06 (d, J=2.80 Hz, 1H), 9.04 (s, 1H), 7.56-7.60 (m, 2H), 7.43-7.48 (m, 2H), 7.38 (d, J=8.00 Hz, 1H), 4.52-4.74 (m, 1H), 4.26-4.37 (m, 1H), 3.65-3.76 (m, 1H), 3.46-3.56 (m, 4H), 3.35-3.41 (m, 1H), 2.96-3.09 (m, 1H), 2.23 (s, 3H), 1.75-2.01 (m, 4H). 4 protons of N-methyl-piperazine moiety were not visible. LC-MS [Method 4]: Rₜ=0.73 min; MS (ESIpos): m/z=501.2 [M+H]⁺.

### Example 81

### [2-Amino-4-(trifluoromethoxy)phenyl]{4-[7-(4-methylpiperazin-1-yl)pyrido[3,2-d]pyrimidin-4-yl]piperidin-1-yl}methanone

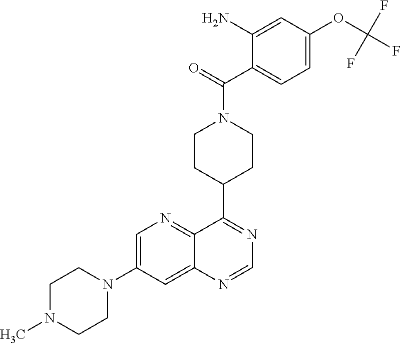 [SMILES: CN1CCN(c2cnc3c(C4CCN(C(=O)c5ccc(OC(F)(F)F)cc5N)CC4)ncnc3c2)CC1]

### Step 1: 3-Amino-5-chloropicolinonitrile

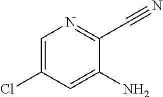

To a solution of 5-chloro-3-nitropicolinonitrile (30 g, 163.4 mmol) in 500 mL of acetic acid were added iron powder (54.8 g, 980.6 mmol). The resulting mixture was stirred at room temperature for 5 hours. Upon completion of the reaction, water was added and the solid was removed by filtration. The filtrate was extracted with ethyl acetate and the combined organic layer was washed with water, brine and dried over anhydrous sodium sulfate. The solvent was removed in vacuo to give 30.0 g (crude) of the title compound as a brown solid. MS (ESIpos): m/z=154 (M+H)⁺. LC-MS [Method 1]: Rₜ=0.92 min.

### Step 2: 3-Amino-5-chloropicolinamide

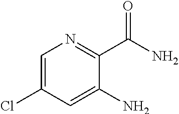 [SMILES: NC(=O)c1ncc(Cl)cc1N]

3-Amino-5-chloropicolinonitrile (25 g, 162.8 mmol) was added 80 mL of concentrated sulfuric Acid and the resulting mixture was stirred at 80° C. for 4 hours. After cooled to room temperature, the resulting mixture was poured into ice cold water and the pH value was adjusted to 12 with saturated sodium hydroxide solution. The precipitated solid was collected by filtration and the filter cake was washed with water and dried in vacuo to give 19 g (77%) of the title compound as a yellow solid. MS (ESIpos): m/z=172 (M+H)⁺. LC-MS [Method 2]: Rₜ=0.75 min.

### Step 3: 7-Chloropyrido[3,2-d]pyrimidin-4-ol

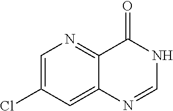 [SMILES: O=c1[nH]cnc2cc(Cl)cnc12]

3-Amino-5-chloropicolinamide (12.1 g, 70.5 mmol) was added to 300 mL of triethyl orthoformate and the resulting solution was stirred at 130° C. for 2 days. After cooled to room temperature, the precipitated solid was collected by filtration and the filter cake was washed with ethyl acetate and dried in vacuo to give 11.4 g (89%) of the title compound as a grey solid. MS (ESIpos): m/z=182 (M+H)⁺. LC-MS [Method 2]: Rₜ=0.55 min.

### Step 4: 4,7-Dichloropyrido[3,2-d]pyrimidine

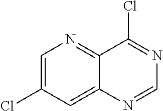 [SMILES: Clc1cnc2c(Cl)ncnc2c1]

To a mixture of 7-chloropyrido[3,2-d]pyrimidin-4-ol (11.4 g, 62.8 mmol) in 500 mL of sulfuryl chloride was added 0.5 mL of N,N-dimethylformamide and the resulting mixture was stirred at reflux for 2 hours. After cooled to room temperature, the solvent was removed in vacuo and the residue was diluted with dichloromethane. Saturated potassium carbonate solution was added at 0° C. to adjust the pH value to 8 and the resulting mixture was extracted with dichloromethane. The combined organic layer was dried over anhydrous sodium sulfate and the solvent was removed in vacuo. The residue was purified by silica gel column chromatography (ethyl acetate:petroleum ether=1:3) to give 8.3 g (64%) of the title compound as a yellow solid. MS (ESIpos): m/z=200 (M+H)⁺. LC-MS [Method 1]: Rₜ=0.74 min.

### Step 5: Tert-Butyl 4-(7-chloropyrido[3,2-d]pyrimidin-4-yl)-5,6-dihydropyridine-1(2H)-carboxylate

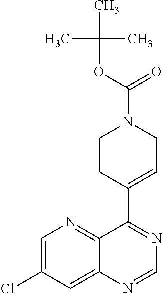 [SMILES: CC(C)(C)OC(=O)N1CC=C(c2ncnc3cc(Cl)cnc23)CC1]

This compound was synthesized by the same method as described in example 1 to give 5.2 g (37%) of the title compound as a yellow solid. MS (ESIpos): m/z=347 (M+H)⁺. LC-MS [Method 2]: Rₜ=2.81 min. ¹H-NMR (400 MHz, DMSO-d₆): δ 1.46 (s, 9H), 2.76-2.78 (m, 2H), 3.59-3.62 (m, 2H), 4.12-4.21 (m, 2H), 7.42 (br, 1H), 8.61 (s, 1H), 9.09 (s, 1H), 9.32 (s, 1H).

### Step 6: Tert-butyl 4-[7-(4-methylpiperazin-1-yl)pyrido[3,2-d]pyrimidin-4-yl]-3,6-dihydropyridine-1(2H)-carboxylate

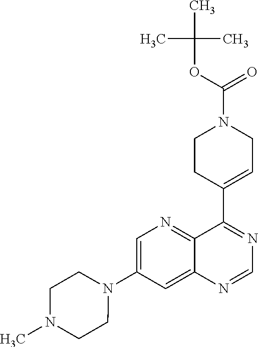 [SMILES: CN1CCN(c2cnc3c(C4=CCN(C(=O)OC(C)(C)C)CC4)ncnc3c2)CC1]

To a solution of tert-butyl 4-(7-chloropyrido[3,2-d]pyrimidin-4-yl)-3,6-dihydropyridine-1(2H)-carboxylate (1.2 g, 3.4 mmol) in 20 mL of toluene were added 1-methyl-piperazine (507 mg, 5.1 mmol), palladium(II) acetate (76 mg, 0.3 mmol), BINAP (420 mg, 0.7 mmol), and cesium carbonate (3.3 g, 10.1 mmol). The resulting mixture was stirred at 100° C. for 14 hours under nitrogen atmosphere. After cooled to room temperature, the solid was removed by filtration and the filtrate was concentrated in vacuo, the residue was purified by silica gel column chromatography (dichloromethane:methanol=20:1) to give 700 mg (crude) of the product as a yellow solid. Then 80 mg of crude product was re-purified by prep-HPLC [Mobile Phase A: Water (0.1% NH₄HCO₃), Mobile Phase B: Acetonitrile; Gradient: 35% B to 60% B in 8 min] to give 32.1 mg of the title compound as a light yellow solid. MS (ESIpos): m/z=411 (M+H)⁺. LC-MS [Method 1]: Rₜ=1.02 min. ¹H-NMR (400 MHz, DMSO-d₆): δ 1.46 (s, 9H), 2.01-2.15 (m, 2H), 2.25 (s, 3H), 2.52-2.53 (m, 2H), 3.49-3.61 (m, 4H), 3.66-3.68 (m, 3H), 3.85-3.86 (m, 2H), 4.85-4.86 (m, 1H), 4.95-5.05 (m, 1H), 6.86-6.93 (m, 1H), 7.37 (s, 1H), 9.00 (s, 1H), 9.04 (s, 1H).

### Step 7: Tert-butyl 4-[7-(4-methylpiperazin-1-yl)pyrido[3,2-d]pyrimidin-4-yl]piperidine-1-carboxylate

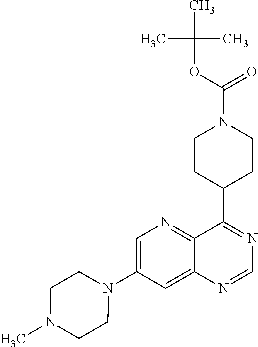 [SMILES: CN1CCN(c2cnc3c(C4CCN(C(=O)OC(C)(C)C)CC4)ncnc3c2)CC1]

This compound was synthesized by the same method as described in example 1 to give 110 mg (20%) of the product as a yellow solid. Then 20 mg of the crude product was re-purified by prep-HPLC [Mobile Phase A: Water (0.1% NH₄HCO₃), Mobile Phase B: Acetonitrile; Gradient: 35% B to 50% B in 8 min] to give 4.4 mg of the title compound as a light yellow solid. MS(ESIpos): m/z=413 (M+H)⁺. LC-MS [Method 1]: Rₜ=0.81 min. ¹H-NMR (400 MHz, DMSO-d₆): δ 1.43 (s, 9H), 1.72-1.82 (m, 4H), 2.24 (s, 3H), 2.47-2.51 (m, 4H), 2.83-2.98 (m, 2H), 3.51-3.53 (m, 4H), 4.09-4.20 (m, 3H), 7.37 (s, 1H), 9.03 (s, 1H), 9.06 (s, 1H).

### Step 8: 7-(4-Methylpiperazin-1-yl)-4-(piperidin-4-yl)pyrido[3,2-d]pyrimidine

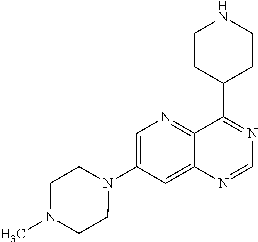 [SMILES: CN1CCN(c2cnc3c(C4CCNCC4)ncnc3c2)CC1]

This compound was synthesized by the same method as described in example 1 to give 50 mg (58%) of the title compound as a yellow solid, MS (ESIpos): m/z=313 (M+H)⁺. LC-MS [Method 1]: Rₜ=0.13 min.

### Step 9: [2-Amino-4-(trifluoromethoxy)phenyl]{4-[7-(4-methylpiperazin-1-yl)pyrido[3,2-d]pyrimidin-4-yl]piperidin-1-yl}methanone

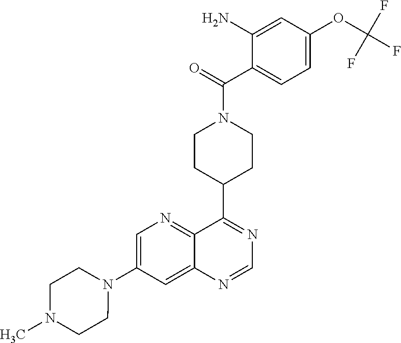 [SMILES: CN1CCN(c2cnc3c(C4CCN(C(=O)c5ccc(OC(F)(F)F)cc5N)CC4)ncnc3c2)CC1]

This compound was synthesized by the same method as described in example 1 to give 13.3 mg (13%) of the title compound as a light yellow solid. MS (ESIpos): m/z=516 (M+H)⁺. LC-MS [Method 1]: Rₜ=0.94 min. ¹H-NMR (400 MHz, DMSO-d₆): δ 1.86-1.87 (m, 4H), 2.64 (s, 3H), 3.03-3.25 (m, 6H), 3.69-3.71 (m, 4H), 3.81-3.82 (m, 2H), 4.30-4.31 (m, 1H), 6.55 (d, 1H), 6.69 (s, 1H), 7.16 (d, 1H), 7.48 (s, 1H), 9.05 (s, 1H), 9.06 (s, 1H).

### Biological Profiling of Cpds

BIOCHEMICAL assay ERK5

Erk5 inhibitory activity of compounds of the present invention was quantified employing the TR-FRET-based Erk5 activity inhibition assay as described in the following paragraphs.

Recombinant fusion protein of N-terminal Glutathion-S-Transferase (GST) and a fragment of human EGFR (amino acids 1-398 of accession number NP_002740.2]), expressed in E. coli, purified via affinity chromatography using Glutathion Sepharose, and subsequently activated with His-tagged MAP2K5 was purchased from Carna Biosciences (product number 04-146) and used as kinase. As substrate for the kinase reaction biotinylated peptide biotin-Ahx-PPGDYSTTPGGTLFSTTPGGTRI (C-terminus in amide form) was used which can be purchased e.g. form the company Biosyntan (Berlin-Buch, Germany). For the assay 50 nl of a 100 fold concentrated solution of the test compound in DMSO was pipetted into either a black low volume 384 well microtiter plate or a black 1536 well microtiter plate (both Greiner Bio-One, Frickenhausen, Germany), 2 μl of a solution of Erk5 in aqueous assay buffer [50 mM Hepes pH 7.0, 15 mMMgCl₂, 1 mM dithiothreitol, 0.5 mM EGTA, 0.05% (w/v) bovine y-globulin (Sigma-Aldrich, #G5009), 0.005% (v/v) NP40 (AppliChem, #A2239)] were added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 417 μM=>final conc. in the 5 μl assay volume is 250 μM) and substrate (1.67 μM=>final conc. in the 5 μl assay volume is 1 μM) in assay buffer and the resulting mixture was incubated for a reaction time of 60 min at 22° C. The concentration of Erk5 was adjusted depending of the activity of the enzyme lot and was chosen appropriate to have the assay in the linear range, a typical concentration was 0.5 μg/ml. The reaction was stopped by the addition of 3 μl of a solution of TR-FRET detection reagents (0.33 μM streptavidine-XL665 [Cisbio Bioassays, Codolet, France] and 1.67 nM anti-4E-BP1 (pT46) antibody from Invitrogen [catalogue no. 700397] and 1.67 nM LANCE EU-W1024 labeled anti-rabbit IgG antibody [Perkin-Elmer, product no. AD0083]) in an aqueous EDTA-solution (83.3 mM EDTA, 0.2% (w/v) bovine serum albumin in 50 mM HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

### Luciferase Reporter Assay

The SN12C-MEF2-luc (clone #37) reporter cell line has been generated by stably transducing SN12C cells with a MEF2-responsive transcription element upstream of a firefly luciferase gene and was used to determine the cellular activity of ERK5 inhibitors. Generation of the poly- and selection of the monoclonal reporter cell lines was carried out at the NMI Natural and Medical Sciences Institute at the university of Tuebingen. This cell line was grown in RPMI 1640 Medium without Phenol Red (Biochrom, #F1275) supplemented with 10% FCS and Glutamax. All cells were grown at 37° C. in a humidified atmosphere with 5% CO2.

On day 1, the cells were seeded in 384-well white plates (Perkin Elmer #6007680) at a density of 10,000 cells per well in 20 μl of culture medium. On day 2, the test compounds were added in serial dilutions using the HP D300 Digital Dispenser and incubated at 37° C. for 16 h.

On day 3, EGF (Invitrogen, #PHG0311L) was added to every well (final concentration=100 ng/ml) and the plates were incubated for additional 2 h at 37° C. Then, 25 μl of ONE-Glo (Promega, #E6120) were added to each well and the plates were incubate for 5 min at RT (shaking). The luminescence signal was read on PHERAStar (BMG Labtech).

IC50s were calculated using the DRC Master Spreadsheet (Bella software). Values obtained for cells treated with EGF and DMSO were defined as the maximum control, while values for cells treated with EGF and 10 μM of XMD8-92 (SN12C-MEF2-luc) were defined as the minimum control (i.e., maximum inhibition).

| | IC₅₀ value | IC₅₀ value Luciferase |
|---|---|---|
| | biochemiocal | reporter assay in |
| Ex. | ERK5 asssay [M] | SN12C cell line [M] |
| 1 | 7.09 E−6 | Nd |
| 2 | 9.31 E−8 | 9.35 E−7 |
| 3 | 9.74 E−6 | Nd |
| 4 | 7.70 E−7 | Nd |
| 5 | 1.14 E−6 | 1.13 E−5 |
| 6 | 2.75 E−6 | Nd |
| 7 | 2.71 E−7 | Nd |
| 8 | 5.25 E−7 | Nd |
| 9 | 6.17 E−6 | Nd |
| 10 | 9.16 E−7 | 3.74 E−6 |
| 11 | 3.44 E−7 | Nd |
| 12 | 1.90 E−7 | Nd |
| 13 | 1.66 E−6 | 1.15 E−5 |
| 14 | 4.10 E−6 | Nd |
| 15 | 3.75 E−7 | Nd |
| 16 | 1.59 E−7 | 1.72 E−6 |
| 17 | 1.11 E−7 | 1.31 E−5 |
| 18 | 3.58 E−7 | Nd |
| 19 | 1.65 E−7 | Nd |
| 20 | 2.81 E−7 | Nd |
| 21 | 2.66 E−6 | Nd |
| 22 | 8.85 E−8 | 1.23 E−6 |
| 23 | 1.12 E−6 | 7.64 E−6 |
| 24 | 9.66 E−6 | Nd |
| 25 | 1.03 E−6 | 6.83 E−6 |
| 26 | 1.63 E−6 | Nd |
| 27 | 4.01 E−7 | 4.91 E−6 |
| 28 | 16.02% @ 20 μM | Nd |
| 29 | 2.06 E−7 | Nd |
| 30 | 7.54 E−7 | Nd |
| 31 | 5.79 E−7 | 1.46 E−5 |
| 32 | 44.19% @ 20 μM | Nd |
| 33 | 2.53 E−7 | Nd |
| 34 | 5.06 E−7 | Nd |
| 35 | 1.46 E−7 | 4.15 E−7 |
| 36 | 1.92 E−7 | Nd |
| 37 | 8.67 E−7 | Nd |
| 38 | 1.27 E−6 | Nd |
| 39 | 3.51 E−7 | Nd |
| 40 | 2.24 E−7 | Nd |
| 41 | 1.09 E−6 | 7.04 E−6 |
| 42 | 9.80 E−7 | Nd |
| 43 | 1.59 E−6 | Nd |
| 44 | 2.24 E−6 | Nd |
| 45 | 3.26 E−6 | Nd |
| 46 | 2.93 E−7 | Nd |
| 47 | 1.10 E−7 | 2.45 E−6 |
| 48 | 2.70 E−7 | Nd |
| 49 | 1.45 E−7 | 3.52 E−7 |
| 50 | 1.67 E−7 | 3.87 E−7 |
| 51 | 4.30 E−8 | 5.94 E−7 |
| 52 | 2.47 E−8 | 5.00 E−7 |
| 53 | 7.81 E−8 | 6.27 E−7 |
| 54 | 1.90 E−7 | 9.42 E−7 |
| 55 | 1.30 E−6 | Nd |
| 56 | 1.98 E−7 | Nd |
| 57 | 1.02 E−6 | 4.01 E−6 |
| 58 | 1.68 E−6 | Nd |
| 59 | 1.13 E−5 | Nd |
| 60 | 1.08 E−7 | 7.08 E−7 |
| 61 | 5.04 E−7 | Nd |
| 62 | 2.74 E−7 | Nd |
| 63 | 2.74 E−7 | Nd |
| 64 | 8.98 E−8 | 6.94 E−7 |
| 65 | 1.72 E−7 | 3.61 E−7 |
| 66 | 5.03 E−8 | 1.64 E−7 |
| 67 | 2.41 E−7 | 6.69 E−7 |
| 68 | 2.17 E−6 | 1.02 E−5 |
| 69 | 6.73 E−6 | Nd |
| 70 | 9.80 E−8 | 5.36 E−7 |
| 71 | 1.53 E−7 | 4.36 E−7 |
| 72 | 6.93 E−8 | 4.15 E−7 |
| 73 | 3.91 E−8 | 2.53 E−7 |
| 74 | 1.69 E−8 | 1.46 E−7 |
| 75 | 6.84 E−8 | 2.34 E−7 |
| 76 | 6.41 E−8 | 6.74 E−7 |
| 77 | 3.03 E−8 | 1.32 E−7 |
| 78 | 3.63 E−8 | 1.03 E−7 |
| 79 | 4.99 E−8 | 1.22 E−7 |
| 80 | 5.35 E−8 | 2.94 E−7 |
| 81 | 3.46 E−8 | 1.15 E−7 |

1. A compound of formula (1): 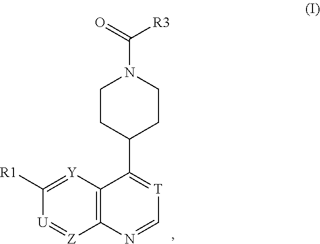
wherein:
T represents CH, CR4, or N;
Y represents CH, CR4, or N;
R1 represents a hydrogen atom, a C₁-C₆-alkyl, —CH3, or C₁-C₆-alkoxy, —OCH₃ group;
U represents CH, CR2, or N;
R2 represents a hydrogen atom, a halogen atom, a bromine atom, a hydroxyl, cyano, a C₁-C₆-alkyl, —CH₃, C₂-C₆-alkenyl, C₃-C₈-cycloalkyl, C₄-C₈-cycloalkenyl, phenyl, 4- to 7-membered heterocycloalkyl, 5- to 8-membered heterocycloalkenyl, heteroaryl, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NR5R6, —C(═O)NH₂, —C(═O)NHCH₃, —C(═O)N(CH₃)₂, —C(═O)NHC₆H₅, —C(═O)OR7-C(═O)OCH₃, —NH₂, —N(H)C(═O)—C₁-C₆-alkyl, —NHC(═O)CH₃, —N(H)—C₃-C₈-cycloalkyl, —N(H)-4- to 7-membered heterocycloalkyl, —N(H)-1-methylpiperidin-4-yl, —N(H)-phenyl, —N(H)S(═O)₂—C₁-C₆-alkyl, —N(H)S(═O)₂CH₃, C₁-C₆-alkoxy which is optionally substituted with a hydroxyl, 4-membered heterocycloalkyl, —NR5R6, or —NHC(═O)—C₁-C₆-alkyl substituent, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃, —O—C₃-C₈-cycloalkyl, —O-(4- to 7-membered heterocycloalkyl), —O-(tetrahydro-2H-pyran-4-yl), C₁-C₆-haloalkoxy, —O(CH₂)CHF₂;
Z represents CH, CR4, —C—C₁-alkyl, or N;
R3 represents a phenyl or pyridyl ring which is optionally substituted once or twice identically or differently with a substituent selected from a halogen atom, a fluorine atom, a chlorine atom, a bromine atom, a hydroxyl, cyano, C₁-C₆-alkyl which is optionally substituted with a C₁-C₆-alkoxy- or C₁-C₆-haloalkoxy-substituent, C1- C₃-C₄-alkyl, trifluoromethyloxymethyl, trifluoroethyloxymethyl group, C₁-C₆-haloalkyl, C₁-trifluoroalkyl, C₂-C₆-alkenyl, C₃-alkenyl, C₃-C₈-cycloalkyl, —C(═O)NR6R7, —NH₂, —NH—C(═O)—C₁-C₆-alkyl, —NHC(═O)CH₃, C₁-C₆-alkoxy which is optionally substituted with a hydroxyl or C₁-C₆-alkyl substituent, C₁- C₂- C₃- C₄-alkoxy, methoxyethoxy, C₁-C₆-haloalkoxy, C₁-trifluoroalkoxy, C₁-difluoroalkoxy, —O—C₃-C₈-cycloalkyl, —O-(4- to 7-membered heterocycloalkyl), C₁-C₆-alkylthio, C₁-C₆-haloalkylthio, C₁-trifluoroalkylthio, or —S(═O)₂—C₁-C₆-alkyl group;
R4 represents a hydrogen atom, or C₁-C₆-alkyl group;
R5, R6 represent, independently from each other
a hydrogen atom, a C₁-C₆-alkyl or phenyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
or a stereoisomer, a tautomer, an N-oxide, a hydrate, a solvate, or a salt thereof, or a mixture thereof.

2. The compound according to claim 1, wherein:
T represents N;
Y represents CH, or N;
R1 represents a C₁-C₆-alkyl, —CH3, or C₁-C₆-alkoxy, —OCH₃ group;
U represents CR2, or N;
R2 represents a hydrogen atom, a halogen atom, a bromine atom, a hydroxyl, cyano, a C₁-C₆-alkyl, —CH₃, 4- to 7-membered heterocycloalkyl, 5- to 8-membered heterocycloalkenyl, heteroaryl, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NR5R6, —C(═O)NH₂, —C(═O)NHCH₃, —C(═O)N(CH₃)₂, —C(═O)NHC₆H₅, —C(═O)OR7-C(═O)OCH₃, —NH₂, —N(H)C(═O)—C₁-C₆-alkyl, —NHC(═O)CH₃, —N(H)-4- to 7-membered heterocycloalkyl, —N(H)-1-methylpiperidin-4-yl, —N(H)S(═O)₂—C₁-C₆-alkyl, —N(H)S(═O)₂CH₃, C₁-C₆-alkoxy which is optionally substituted with a hydroxyl, 4-membered heterocycloalkyl, —NR5R6, or —NHC(═O)—C₁-C₆-alkyl substituent, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃, —O-(4- to 7-membered heterocycloalkyl), —O-(tetrahydro-2H-pyran-4-yl), C₁-C₆-haloalkoxy, —O(CH₂)CHF₂;
Z represents CH, CR4, —C—C₁-alkyl, or N;
R3 represents a phenyl or pyridyl ring which is optionally substituted once or twice identically or differently with a substituent selected from a halogen atom, a fluorine atom, a chlorine atom, a bromine atom, cyano, C₁-C₆-alkyl which is optionally substituted with a C₁-C₆-alkoxy- or C₁-C₆-haloalkoxy-substituent, C1-C₃-C₄-alkyl, trifluoromethyloxymethyl, trifluoroethyloxymethyl group, C₁-C₆-haloalkyl, C₁-trifluoroalkyl, C₂-C₆-alkenyl, C₃-alkenyl, —NH₂, —NH—C(═O)—C₁-C₆-alkyl, —NHC(═O)CH₃, C₁-C₆-alkoxy which is optionally substituted with a C₁-C₆-alkyl substituent, C₁- C₂- C₃- C₄-alkoxy, methoxyethoxy, C₁-C₆-haloalkoxy, C₁-trifluoroalkoxy, C₁-difluoroalkoxy, or C₁-C₆-haloalkylthio, C₁-trifluoroalkylthio group;
R4 represents a hydrogen atom, or C₁-C₆-alkyl group;
R5, R6 represent, independently from each other
a hydrogen atom, or a C₁-C₆-alkyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
or a stereoisomer, a tautomer, an N-oxide, a hydrate, a solvate, or a salt thereof, or a mixture thereof.

3. The compound according to claim 1, wherein:
T represents N;
Y represents CH, or N;
R1 represents a —CH3, or —OCH₃ group;
U represents CR2, or N;
R2 represents a hydrogen atom, a bromine atom, a hydroxyl, cyano, —CH₃, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NH₂, —C(═O)NHCH₃, —C(═O)N(CH₃)₂, —C(═O)NHC₆H₅, —C(═O)OCH₃, —NH₂, —NHC(═O)CH₃, —N(H)-1-methylpiperidin-4-yl, —N(H)S(═O)₂CH₃, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃—O-(tetrahydro-2H-pyran-4-yl), or —O(CH₂)CHF₂ group;
Z represents CH, —C—C₁-alkyl, or N;
R3 represents a phenyl or pyridyl ring which is optionally substituted once or twice identically or differently with a substituent selected from a fluorine atom, a chlorine atom, a bromine atom, cyano, C₁-, C₃, or C₄-alkyl, trifluoromethyloxymethyl, trifluoroethyloxymethyl group, C₁-trifluoroalkyl, C₃-alkenyl, —NH₂, —NHC(═O)CH₃, C₁- C₂- C₃- C₄-alkoxy, methoxyethoxy, C₁-trifluoroalkoxy, C₁-difluoroalkoxy, or C₁-trifluoroalkylthio group;
R4 represents a hydrogen atom, or C₁-C₆-alkyl group;
R5, R6 represent, independently from each other
a hydrogen atom, or a C₁-C₆-alkyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
or a stereoisomer, a tautomer, an N-oxide, a hydrate, a solvate, or a salt thereof, or a mixture thereof.

4. The compound according to claim 1, wherein:
T represents N;
Y represents CH, or N;
R1 represents a —CH3, or —OCH₃ group;
U represents CR2;
R2 represents a hydrogen atom, a bromine atom, a hydroxyl, cyano, —CH₃, 3,6-dihydro-2H-pyran-4-yl-, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NH₂, —C(═O)N(CH₃)₂, —C(═O)OCH₃, —NH₂, —N(H)-1-methylpiperidin-4-yl, —N(H)S(═O)₂CH₃, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃—O-(tetrahydro-2H-pyran-4-yl), or —O(CH₂)CHF₂ group;
Z represents CH, or —C—C₁-alkyl group;
R3 represents a phenyl ring which is optionally substituted once or twice identically or differently with a substituent selected from a fluorine atom, a chlorine atom, a bromine atom, C₁-, or C₃-alkyl, C₁-trifluoroalkyl, C₃-alkenyl, —NH₂, —NHC(═O)CH₃, C₁- C₂- C₃- C₄-alkoxy, C₁-difluoroalkoxy, C₁-trifluoroalkoxy, or C₁-trifluoroalkylthio group;
R4 represents a hydrogen atom, or C₁-C₆-alkyl group;
R5, R6 represent, independently from each other
a hydrogen atom, or a C₁-C₆-alkyl group;
or
R5, R6, together with the nitrogen atom to which they are attached, represent a 4- to 7-membered heterocycloalkyl group which is optionally substituted with an oxo (═O) substituent;
R7 represents a hydrogen atom, or a C₁-C₆-alkyl group;
or a stereoisomer, a tautomer, an N-oxide, a hydrate, a solvate, or a salt thereof, or a mixture thereof.

5. The compound according to claim 1 which is selected from the group consisting of:
[4-(6,7-Dimethoxyquinazolin-4-yl)piperidin-1-yl][3-(trifluoromethoxy)phenyl]methanone;
[4-(6,7-Dimethoxyquinazolin-4-yl)piperidin-1-yl][2-fluoro-4-(trifluoromethoxy)phenyl]methanone;
4-{[4-(6,7-Dimethoxyquinazolin-4-yl)piperidin-1-yl]carbonyl}benzonitrile;
4-[1-(4-Chlorobenzoyl)piperidin-4-yl]-6,7-dimethoxyquinazoline;
[4-(6,7-Dimethoxyquinazolin-4-yl)piperidin-1-yl]{4-[(trifluoromethoxy)methyl]phenyl}methanone;
4-[1-(4-Fluorobenzoyl)piperidin-4-yl]-6,7-dimethoxyquinazoline;
6,7-Dimethoxy-4-{1-[4-(trifluoromethoxy)benzoyl]-piperidin-4-yl}quinazoline;
6,7-Dimethoxy-4-{1-[4-(trifluoromethyl)benzoyl]-piperidin-4-yl}quinazoline;
[4-(6,7-Dimethoxyquinazolin-4-yl)piperidin-1-yl]-(phenyl)methanone;
[4-(6,7-Dimethoxyquinazolin-4-yl)piperidin-1-yl][4-(propan-2-yloxy)phenyl]methanone;
[4-(6,7-Dimethoxyquinazolin-4-yl)piperidin-1-yl](4-methoxyphenyl)methanone;
(4-Bromophenyl)[4-(6,7-dimethoxyquinazolin-4-yl)piperidin-1-yl]methanone;
[4-(6,7-Dimethoxyquinazolin-4-yl)piperidin-1-yl](4-methylphenyl)methanone;
(4-Tert-butylphenyl)[4-(6,7-dimethoxyquinazolin-4-yl)piperidin-1-yl]methanone;
4-(6,7-Dimethoxyquinazolin-4-yl)piperidin-1-yl](4-ethoxyphenyl)methanone;
[4-(6,7-Dimethoxyquinazolin-4-yl)piperidin-1-yl]{4-[(trifluoromethyl)sulfanyl]-phenyl}methanone;
(4-Butoxyphenyl)[4-(6,7-dimethoxyquinazolin-4-yl)piperidin-1-yl]methanone;
[4-(6,7-dimethoxyquinazolin-4-yl)piperidin-1-yl][4-(propan-2-yl)phenyl]-methanone;
[4-(Difluoromethoxy)phenyl][4-(6,7-dimethoxy-quinazolin-4-yl)piperidin-1-yl]methanone;
[4-(6,7-dimethoxyquinazolin-4-yl)piperidin-1-yl](4-propylphenyl)methanone;
[4-(6,7-Dimethoxyquinazolin-4-yl)piperidin-1-yl]{4-[(2,2,2-trifluoroethoxy)methyl]phenyl}-methanone;
[4-(6,7-Dimethoxyquinazolin-4-yl)piperidin-1-yl][3-fluoro-4-(trifluoromethoxy)phenyl]methanone;
[4-(6,7-Dimethoxyquinazolin-4-yl)piperidin-1-yl][6-(trifluoromethoxy)pyridin-3-yl]methanone;
[4-(6,7-Dimethoxyquinazolin-4-yl)piperidin-1-yl][4-(2-methoxyethoxy)phenyl]methanone;
(4-Tert-Butoxyphenyl)[4-(6,7-dimethoxy-quinazolin-4-yl)piperidin-1-yl]methanone;
[4-(6,7-Dimethoxyquinazolin-4-yl)piperidin-1-yl][5-(trifluoromethoxy)pyridin-2-yl]methanone;
[4-(6,7-dimethoxyquinazolin-4-yl)piperidin-1-yl][4-(prop-2-en-1-yloxy)phenyl]methanone;
[4-(7-Methylquinazolin-4-yl)piperidin-1-yl][4-(trifluoromethoxy)phenyl]methanone;
[4-(6-Methylquinazolin-4-yl)piperidin-1-yl][4-(trifluoromethoxy)phenyl]methanone;
Methyl 4-{1-[4-(trifluoromethoxy)benzoyl]piperidin-4-yl}quinazoline-7-carboxylate;
4-{1-[4-(Trifluoromethoxy)benzoyl]piperidin-4-yl}quinazoline-7-carboxamide;
4-{1-[4-(Trifluoromethoxy)benzoyl]piperidin-4-yl}quinazoline-7-carbonitrile;
[4-(7-Methoxyquinazolin-4-yl)piperidin-1-yl][4-(trifluoromethoxy)phenyl]methanone;
[4-(7-Hydroxyquinazolin-4-yl)piperidin-1-yl][4-(trifluoromethoxy)phenyl]methanone;
[4-(7-Aminoquinazolin-4-yl)piperidin-1-yl][4-(trifluoromethoxy)phenyl]methanone;
N-(4-{1-[4-(Trifluoromethoxy)benzoyl]piperidin-4-yl}quinazolin-7-yl)acetamide;
N-(4-{1-[4-(Trifluoromethoxy)benzoyl]piperidin-4-yl}quinazolin-7-yl)methanesulfonamide;
[4-(7-Bromoquinazoline-4-yl)piperidin-1-yl][4-(trifluoromethoxy)phenyl]methanone;
N-Methyl-4-{1-[4-(trifluoromethoxy)benzoyl]-piperidin-4-yl}quinazoline-7-carboxamide;
N,N-Dimethyl-4-{1-[4-(trifluoromethoxy)benzoyl]-piperidin-4-yl}quinazoline-7-carboxamide;
Pyrrolidin-1-yl(4-{1-[4-(trifluoromethoxy)benzoyl]-piperidin-4-yl}quinazolin-7-yl)methanone;
Morpholin-4-yl(4-{1-[4-(trifluoromethoxy)benzoyl]-piperidin-4-yl}quinazolin-7-yl)methanone;
N-Phenyl-4-{1-[4-(trifluoromethoxy)benzoyl]-piperidin-4-yl}quinazoline-7-carboxamide;
{4-[7-(2-Methylpropoxy)quinazolin-4-yl]piperidin-1-yl}[4-(trifluoromethoxy)phenyl]methanone;
{4-[7-(2-Hydroxyethoxy)quinazolin-4-yl]piperidin-1-yl}[4-(trifluoromethoxy)phenyl]methanone;
{4-[7-(Oxetan-3-ylmethoxy)quinazolin-4-yl]piperidin-1-yl}[4-(trifluoromethoxy)phenyl]-methanone;
{4-[7-(Tetrahydro-2H-pyran-4-yloxy)quinazolin-4-yl]piperidin-1-yl}[4-(trifluoromethoxy)-phenyl]methanone;
N-{2-[(4-{1-[4-(Trifluoromethoxy)benzoyl] piperidin-4-yl}quinazolin-7-yl)oxy]ethyl}acetamide [4-(7-Cyclopropylquinazolin-4-yl)piperidin-1-yl][4-(trifluoromethoxy)phenyl]methanon;
{4-[7-(3,6-Dihydro-2H-pyran-4-yl)quinazolin-4-yl]-piperidin-1-yl}[4-(trifluoromethoxy)phenyl]methanone;
{4-[7-(1H-Pyrazol-5-yl)quinazolin-4-yl]piperidin-1-yl}[4-(trifluoromethoxy)phenyl]methanone;
1-(4-{1-[4-(Trifluoromethoxy)benzoyl]piperidin-4-yl}quinazolin-7-yl)pyrrolidin-2-one;
1-(4-{1-[4-(Trifluoromethoxy)benzoyl]piperidin-4-yl}quinazolin-7-yl)piperidin-2-one;
Formic acid—(4-{7-[2-(dimethylamino)ethoxy]quinazolin-4-yl}piperidin-1-yl)[4-(trifluoromethoxy)phenyl]methanone (1:1);
[4-(6-Methoxyquinazolin-4-yl)piperidin-1-yl][4-(trifluoromethoxy)phenyl]methanone [4-(Quinazolin-4-yl)piperidin-1-yl][4-(trifluoromethoxy)phenyl]methanone;
[4-(7-Methoxyquinolin-4-yl)piperidin-1-yl][4-(trifluoromethoxy)phenyl]methanone;
[4-(7-Methoxyquinazolin-4-yl)piperidin-1-yl][2-methyl-4-(trifluoromethoxy)phenyl]methanone;
[4-(7-Methoxyquinazolin-4-yl)piperidin-1-yl][3-methyl-4-(trifluoromethoxy)phenyl]methanone;
{4-[7-(2-Hydroxy-2-methylpropoxy)quinazolin-4-yl]piperidin-1-yl}[4-(trifluoromethoxy)phenyl]methanone;
{4-[7-(2,2-Difluoroethoxy)quinazolin-4-yl]piperidin-1-yl}[4-(trifluoromethoxy)phenyl]methanone;
[3-Amino-4-(trifluoromethoxy)phenyl][4-(7-methoxyquinazolin-4-yl)piperidin-1-yl]methanone;
[2-Amino-4-(trifluoromethoxy)phenyl][4-(7-methoxyquinazolin-4-yl)piperidin-1-yl]methanone;
N-[2-{[4-(7-Methoxyquinazolin-4-yl)piperidin-1-yl]carbonyl}-5-(trifluoromethoxy)phenyl]acetamide;
4-(6-Methoxypyrido[3,4-d]pyrimidin-4-yl)piperidin-1-yl][4-(trifluoromethoxy)-phenyl]methanone;
[4-(7-Methoxypyrido[2,3-d]pyrimidin-4-yl)piperidin-1-yl][4-(trifluoromethoxy)phenyl]methanone;
[4-(7-Methoxypyrido[3,2-d]pyrimidin-4-yl)piperidin-1-yl][4-(trifluoromethoxy)phenyl]methanone;
(4-{7-[(1-Methylpiperidin-4-yl)amino]pyrido[3,2-d]pyrimidin-4-yl}piperidin-1-yl)[4-(trifluoro-methoxy)phenyl]methanone;
{4-[7-(3,3-Difluoropyrrolidin-1-yl)pyrido[3,2-d]pyrimidin-4-yl]piperidin-1-yl}[4-(trifluoromethoxy)phenyl]methanone;
{4-[7-(4-Amino-4-methylpiperidin-1-yl)pyrido[3,2-d]pyrimidin-4-yl]piperidin-1-yl}[4-(trifluoromethoxy)phenyl]methanone;
{4-[7-(Morpholin-4-yl)pyrido[3,2-d]pyrimidin-4-yl]piperidin-1-yl}[4-(trifluoromethoxy)-phenyl]methanone;
(4-{7-[3-Hydroxy-3-methylpyrrolidin-1-yl]pyrido[3,2-d]pyrimidin-4-yl}piperidin-1-yl)[4-(trifluoromethoxy)phenyl]methanone;
(4-{7-[3-(Dimethylamino)pyrrolidin-1-yl]pyrido[3,2-d]pyrimidin-4-yl}piperidin-1-yl)[4-(trifluoromethoxy)phenyl]methanone;
{4-[7-(4,4-Difluoropiperidin-1-yl)pyrido[3,2-d]pyrimidin-4-yl]piperidin-1-yl}[4-(trifluoromethoxy)phenyl]methanone;
{4-[7-(2,2-Dimethylmorpholin-4-yl)pyrido[3,2-d]pyrimidin-4-yl]piperidin-1-yl}[4-(trifluoromethoxy)phenyl]methanone;
{4-[7-(4-Methoxypiperidin-1-yl)pyrido[3,2-d]pyrimidin-4-yl]piperidin-1-yl}[4-(trifluoromethoxy)phenyl]methanone;
{4-[7-(4-Methylpiperazin-1-yl)pyrido[3,2-d]pyrimidin-4-yl]piperidin-1-yl}[4-(trifluoromethoxy)phenyl]methanone; and
[2-Amino-4-(trifluoromethoxy)phenyl]{4-[7-(4-methylpiperazin-1-yl)pyrido[3,2-d]pyrimidin-z yl]piperidin-1-yl}methanone;
or a stereoisomer, a tautomer, an N-oxide, a hydrate, a solvate, or a salt thereof, or a mixture thereof.

6. The compound according to claim 1, which is selected from the group consisting of:
[4-(6,7-Dimethoxyquinazolin-4-yl)piperidin-1-yl][2-fluoro-4-(trifluoromethoxy)phenyl]methanone
4-[1-(4-Chlorobenzoyl)piperidin-4-yl]-6,7-dimethoxyquinazoline;
6,7-Dimethoxy-4-{1-[4-(trifluoromethoxy)benzoyl]-piperidin-4-yl}quinazoline;
6,7-Dimethoxy-4-{1-[4-(trifluoromethyl)benzoyl]-piperidin-4-yl}quinazoline;
[4-(6,7-Dimethoxyquinazolin-4-yl)piperidin-1-yl][4-(propan-2-yloxy)phenyl]methanone;
[4-(6,7-Dimethoxyquinazolin-4-yl)piperidin-1-yl](4-methoxyphenyl)methanone;
(4-Bromophenyl)[4-(6,7-dimethoxyquinazolin-4-yl)piperidin-1-yl]methanone;
4-(6,7-Dimethoxyquinazolin-4-yl)piperidin-1-yl](4-ethoxyphenyl)methanone;
[4-(6,7-Dimethoxyquinazolin-4-yl)piperidin-1-yl]{4-[(trifluoromethyl)sulfanyl]-phenyl}methanone;
(4-Butoxyphenyl)[4-(6,7-dimethoxyquinazolin-4-yl)piperidin-1-yl]methanone;
[4-(6,7-dimethoxyquinazolin-4-yl)piperidin-1-yl][4-(propan-2-yl)phenyl]-methanone;
[4-(Difluoromethoxy) phenyl][4-(6,7-dimethoxy-quinazolin-4-yl)piperidin-1-yl]methanone;
[4-(6,7-dimethoxyquinazolin-4-yl)piperidin-1-yl](4-propylphenyl)methanone;
[4-(6,7-Dimethoxyquinazolin-4-yl)piperidin-1-yl][3-fluoro-4-(trifluoromethoxy)phenyl]methanone;
[4-(6,7-dimethoxyquinazolin-4-yl)piperidin-1-yl][4-(prop-2-en-1-yloxy)phenyl]methanone;
[4-(7-Methylquinazolin-4-yl)piperidin-1-yl][4-(trifluoromethoxy)phenyl]methanone;
[4-(6-Methylquinazolin-4-yl)piperidin-1-yl][4-(trifluoromethoxy)phenyl]methanone;
Methyl 4-{1-[4-(trifluoromethoxy)benzoyl]piperidin-4-yl}quinazoline-7-carboxylate;
4-{1-[4-(Trifluoromethoxy)benzoyl]piperidin-4-yl}quinazoline-7-carboxamide;
4-{1-[4-(Trifluoromethoxy)benzoyl]piperidin-4-yl}quinazoline-7-carbonitrile;
[4-(7-Methoxyquinazolin-4-yl)piperidin-1-yl][4-(trifluoromethoxy)phenyl]methanone;
[4-(7-Hydroxyquinazolin-4-yl)piperidin-1-yl][4-(trifluoromethoxy)phenyl]methanone;
[4-(7-Aminoquinazolin-4-yl)piperidin-1-yl][4-(trifluoromethoxy)phenyl]methanone;
N-(4-{1-[4-(Trifluoromethoxy)benzoyl]piperidin-4-yl}quinazolin-7-yl)methanesulfonamide;
[4-(7-Bromoquinazoline-4-yl)piperidin-1-yl][4-(trifluoromethoxy)phenyl]methanone;
N,N-Dimethyl-4-{1-[4-(trifluoromethoxy)benzoyl]-piperidin-4-yl}quinazoline-7-carboxamide;
{4-[7-(2-Methylpropoxy)quinazolin-4-yl]piperidin-1-yl}[4-(trifluoromethoxy)phenyl]methanone;
{4-[7-(2-Hydroxyethoxy)quinazolin-4-yl]piperidin-1-yl}[4-(trifluoromethoxy)phenyl]methanone;
{4-[7-(Oxetan-3-ylmethoxy)quinazolin-4-yl]piperidin-1-yl}[4-(trifluoromethoxy)phenyl]-methanone;
{4-[7-(Tetrahydro-2H-pyran-4-yloxy)quinazolin-4-yl]piperidin-1-yl}[4-(trifluoromethoxy)-phenyl]methanone;
N-{2-[(4-{1-[4-(Trifluoromethoxy)benzoyl] piperidin-4-yl}quinazolin-7-yl)oxy]ethyl}acetamide;
[4-(7-Cyclopropylquinazolin-4-yl)piperidin-1-yl][4-(trifluoromethoxy)phenyl]methanon;
{4-[7-(3,6-Dihydro-2H-pyran-4-yl)quinazolin-4-yl]-piperidin-1-yl}[4-(trifluoromethoxy)phenyl]methanone;
{4-[7-(1H-Pyrazol-5-yl)quinazolin-4-yl]piperidin-1-yl}[4-(trifluoromethoxy)phenyl]methanone;
1-(4-{1-[4-(Trifluoromethoxy)benzoyl]piperidin-4-yl}quinazolin-7-yl)pyrrolidin-2-one;
Formic acid—(4-{7-[2-(dimethylamino)ethoxy]quinazolin-4-yl}piperidin-1-yl)[4-(trifluoromethoxy)phenyl]methanone (1:1);
[4-(7-Methoxyquinolin-4-yl)piperidin-1-yl][4-(trifluoromethoxy)phenyl]methanone;
[4-(7-Methoxyquinazolin-4-yl)piperidin-1-yl][2-methyl-4-(trifluoromethoxy)phenyl]methanone;
[4-(7-Methoxyquinazolin-4-yl)piperidin-1-yl][3-methyl-4-(trifluoromethoxy)phenyl]methanone;
{4-[7-(2-Hydroxy-2-methylpropoxy)quinazolin-4-yl]piperidin-1-yl}[4-(trifluoromethoxy)phenyl]methanone;
{4-[7-(2,2-Difluoroethoxy)quinazolin-4-yl]piperidin-1-yl}[4-(trifluoromethoxy)phenyl]methanone;
[3-Amino-4-(trifluoromethoxy)phenyl][4-(7-methoxyquinazolin-4-yl)piperidin-1-yl]methanone;
[2-Amino-4-(trifluoromethoxy)phenyl][4-(7-methoxyquinazolin-4-yl)piperidin-1-yl]methanone;
N-[2-{[4-(7-Methoxyquinazolin-4-yl)piperidin-1-yl]carbonyl}-5-(trifluoromethoxy)phenyl]acetamide;
[4-(7-Methoxypyrido[3,2-d]pyrimidin-4-yl)piperidin-1-yl][4-(trifluoromethoxy)phenyl]methanone;
(4-{7-[(1-Methylpiperidin-4-yl)amino]pyrido[3,2-d]pyrimidin-4-yl}piperidin-1-yl)[4-(trifluoro-methoxy)phenyl]methanone;
{4-[7-(3,3-Difluoropyrrolidin-1-yl)pyrido[3,2-d]pyrimidin-4-yl]piperidin-1-yl}[4-(trifluoromethoxy)phenyl]methanone;
{4-[7-(4-Amino-4-methylpiperidin-1-yl)pyrido[3,2-d]pyrimidin-4-yl]piperidin-1-yl}[4-(trifluoromethoxy)phenyl]methanone;
{4-[7-(Morpholin-4-yl)pyrido[3,2-d]pyrimidin-4-yl]piperidin-1-yl}[4-(trifluoromethoxy)-phenyl]methanone;
(4-{7-[(3S)-3-Hydroxy-3-methylpyrrolidin-1-yl]pyrido[3,2-d]pyrimidin-4-yl}piperidin-1-yl)[4-(trifluoromethoxy)phenyl]methanone;
(4-{7-[3-(Dimethylamino)pyrrolidin-1-yl]pyrido[3,2-d]pyrimidin-4-yl}piperidin-1-yl)[4-(trifluoromethoxy)phenyl]methanone;
{4-[7-(4,4-Difluoropiperidin-1-yl)pyrido[3,2-d]pyrimidin-4-yl]piperidin-1-yl}[4-(trifluoromethoxy)phenyl]methanone;
{4-[7-(2,2-Dimethylmorpholin-4-yl)pyrido[3,2-d]pyrimidin-4-yl]piperidin-1-yl}[4-(trifluoromethoxy)phenyl]methanone;
{4-[7-(4-Methoxypiperidin-1-yl)pyrido[3,2-d]pyrimidin-4-yl]piperidin-1-yl}[4-(trifluoromethoxy)phenyl]methanone;
{4-[7-(4-Methylpiperazin-1-yl)pyrido[3,2-d]pyrimidin-4-yl]piperidin-1-yl}[4-(trifluoromethoxy)phenyl]methanone; and
[2-Amino-4-(trifluoromethoxy)phenyl]{4-[7-(4-methylpiperazin-1-yl)pyrido[3,2-d]pyrimidin-4-yl]piperidin-1-yl}methanone;
or a stereoisomer, a tautomer, an N-oxide, a hydrate, a solvate, or a salt thereof, or a mixture thereof.

7. A method of preparing a compound according to claim 1, said method comprising allowing an intermediate compound of formula (2): 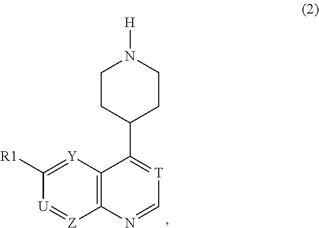
to react with a compound of formula (3): 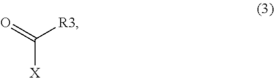
wherein, X is a leaving group optionally a halogen atom, optionally Br, Cl or I optionally, a hydroxyl group, a C₁-C₆-alkyl-O— group, a C₁-C₆-alkyl-C(═O)—O— group, or an aryl-C(═O)—O— group,
optionally in the presence of a base, optionally triethylamine, pyridine, N-ethyl-N,N-diisopropylamine,
optionally in a solvent, optionally an aprotic polar or a non-polar solvent optionally acetonitrile, dichloromethane, 1,2 dichloroethane, chloroform, N,N-dimethylformamide (DMF), 1-methyl-pyrrolidin-2-one (NMP), or mixture thereof,
optionally at ambient or an elevated temperature,
optionally in the presence of a catalyst, optionally N,N-dimethylaminopyridine
thereby giving a compound of formula (I):

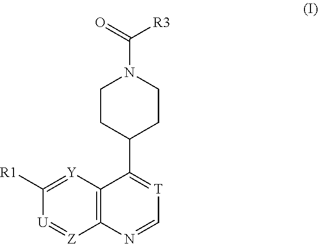

8. A compound of claim 1 adapted for use in treatment or prophylaxis of a disease.

9. A pharmaceutical composition comprising a compound of claim 1 and one or more pharmaceutically acceptable excipients.

10. A pharmaceutical combination comprising:
one or more first active ingredients, comprising a compound according to claim 1, and
one or more further active ingredients, optionally anti-cancer agents.

11. A product comprising a compound according to claim 1 for treatment or prophylaxis of a disease.

12. A product comprising a compound according to claim 1 adapted for treatment or prophylaxis of a disease.

13. The compound according to claim 8 wherein the disease is a cancer, optionally a breast cancer, optionally invasive ductal carcinoma, invasive lobular carcinoma, ductal carcinoma in situ, and lobular carcinoma in situ; a liver cancer, optionally hepatocellular carcinoma, cholangiocarcinoma, or mixed hepatocellular cholangiocarcinoma; or a kidney cancer.

14. A compound of formula (2′): 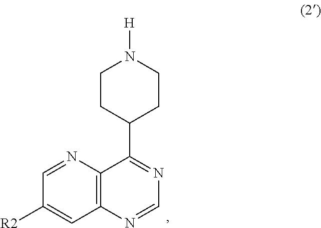
wherein R2
represents a hydrogen atom, a halogen atom, a bromine atom, a hydroxyl, cyano, a C₁-C₆-alkyl, —CH₃, C₂-C₆-alkenyl, C₃-C₈-cycloalkyl, C₄-C₈-cycloalkenyl, phenyl, 4- to 7-membered heterocycloalkyl, 5- to 8-membered heterocycloalkenyl, heteroaryl, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NR5R6, —C(═O)NH₂, —C(═O)NHCH₃, —C(═O)N(CH₃)₂, —C(═O)NHC₆H₅, —C(═O)OR7-C(═O)OCH₃, —NH₂, —N(H)C(═O)—C₁-C₆-alkyl, —NHC(═O)CH₃, —N(H)—C₃-C₈-cycloalkyl, —N(H)-4- to 7-membered heterocycloalkyl, —N(H)-1-methylpiperidin-4-yl, —N(H)-phenyl, —N(H)S(═O)₂—C₁-C₆-alkyl, —N(H)S(═O)₂CH₃, C₁-C₆-alkoxy which is optionally substituted with a hydroxyl, 4-membered heterocycloalkyl, —NR5R6, or —NHC(═O)—C₁-C₆-alkyl substituent, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃, —O—C₃-C₈-cycloalkyl, —O-(4- to 7-membered heterocycloalkyl), —O-(tetrahydro-2H-pyran-4-yl), C₁-C₆-haloalkoxy, —O(CH₂)CHF₂.

15. A product comprising a compound of formula (2′): 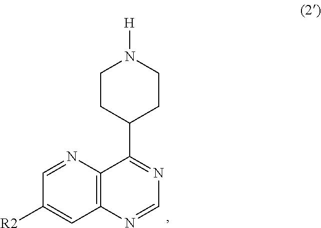
wherein R2
represents a hydrogen atom, a halogen atom, a bromine atom, a hydroxyl, cyano, a C₁-C₆-alkyl, —CH₃, C₂-C₆-alkenyl, C₃-C₈-cycloalkyl, C₄-C₈-cycloalkenyl, phenyl, 4- to 7-membered heterocycloalkyl, 5- to 8-membered heterocycloalkenyl, heteroaryl, 3,6-dihydro-2H-pyran-4-yl-, pyrrolidin-1-yl, pyrrolidin-2-onyl-, (1H-pyrazol-5-yl)-, 3-hydroxy-3-methylpyrrolidin-1-yl-, N-1-methylpiperidin-4-yl-, morpholin-4-yl-, (3,3-difluoropyrrolidin-1-yl-, 1-piperidin-4-onyl-, 4-amino-4-methylpiperidin-1-yl-, 4,4-difluoropiperidin-1-yl-, 2,2-dimethylmorpholin-4-yl-, 4-methoxypiperidin-1-yl-, 4-methylpiperazin-1-yl-, 3-(dimethylamino)pyrrolidin-1-yl-, —C(═O)NR5R6, —C(═O)NH₂, —C(═O)NHCH₃, —C(═O)N(CH₃)₂, —C(═O)NHC₆H₅, —C(═O)OR7-C(═O)OCH₃, —NH₂, —N(H)C(═O)—C₁-C₆-alkyl, —NHC(═O)CH₃, —N(H)—C₃-C₈-cycloalkyl, —N(H)-4- to 7-membered heterocycloalkyl, —N(H)-1-methylpiperidin-4-yl, —N(H)-phenyl, —N(H)S(═O)₂—C₁-C₆-alkyl, —N(H)S(═O)₂CH₃, C₁-C₆-alkoxy which is optionally substituted with a hydroxyl, 4-membered heterocycloalkyl, —NR5R6, or —NHC(═O)—C₁-C₆-alkyl substituent, —OCH₃, —OCH₂CH(CH₃)₂, —OCH₂CH₂OH, —OCH₂C(CH₃)₂OH, —OCH₂-(oxetan-3-yl), —OCH₂CH₂N(CH₃)₂, —OCH₂CH₂NHC(═O)CH₃, —O—C₃-C₈-cycloalkyl, —O-(4- to 7-membered heterocycloalkyl), —O-(tetrahydro-2H-pyran-4-yl), C₁-C₆-haloalkoxy, —O(CH₂)CHF₂,
for preparation of a compound according to claim 1.
